(19) United States
(12) Patent Application Publication
(10) Pub. No.: US 2023/0346906 A1
(43) Pub. Date: Nov. 2, 2023

(54) CANCER TREATMENT STRATEGIES USING ARENAVIRUS VECTORS

(71) Applicant: Hookipa Biotech GmbH, Vienna (AT)

(72) Inventors: Igor MATUSHANSKY, Brooklyn, NY (US); Andy HWANG, Jersey City, NJ (US); Kianoosh KATCHAR, Hoboken, NJ (US); Donna EDWARDS, New York, NY (US); Henning LAUTERBACH, Eching (DE); Michael SCHWENDINGER, Neusiedl am See (AT); Klaus ORLINGER, Vienna (AT); Sarah SCHMIDT, Vienna (AT); Ursula BERKA, Baden (AT); Katia SCHLIENGER, New York, NY (US); Corinne IACOBUCCI, Hampton, NJ (US)

(21) Appl. No.: 17/928,098

(22) PCT Filed: May 12, 2021
(86) PCT No.: PCT/EP2021/062728

§ 371 Date: Nov. 28, 2022

Related U.S. Application Data
(60) Provisional application No. 63/032,362, filed on May 29, 2020.
(60) Provisional application No. 63/173,155, filed on Apr. 9, 2021.
(60) Provisional application No. 63/175,842, filed on Apr. 16, 2021.

(51) Int. Cl. A61K 39/12; C07K 16/28; A61P 37/04

(52) U.S. Cl. CPC A61K 39/12; C07K 16/2818; A61P 37/04; A61K 2039/545

(57) ABSTRACT

The present application relates generally to cancer treatment strategies using arenavirus particles. The treatment strategies described herein are for treating cancer, including head and neck squamous cell carcinoma, using tri-segmented arenavirus particles encoding a human papillomavirus (HPV) antigen. The treatment strategies described herein can include administration of an immune checkpoint inhibitor.

This application claims the benefit of priority to U.S. Ser. No. 63/032,362 filed May 29, 2020, U.S. Ser. No. 63/173,155 filed Apr. 9, 2021, and U.S. Ser. No. 63/175,842 filed Apr. 16, 2021, each of which is incorporated herein by reference in its entirety.

The present application relates generally to cancer treatment strategies using arenavirus particles, and more specifically to specific treatment strategies for treating cancer, including head and neck squamous cell carcinoma, using tri-segmented arenavirus particles encoding an HPV antigen, and in some aspects administration of an immune checkpoint inhibitor.

### BACKGROUND OF THE DISCLOSURE

Human Papillomavirus 16 (HPV16) infection is associated with a substantial and rising proportion of cancers worldwide, such as cervical, head and neck, vaginal, and anal cancers (see de Martel C, et al. Int J Cancer. 2017; 141:664-670). Treatment options are limited for patients with HPV16⁺ recurrent or metastatic cancers, and the likelihood of long-term survival is low. The generation and maintenance of the HPV16⁺ malignant state requires the stable expression of HPV16-specific E7 and E6 oncogenes, which have been shown to drive the cells' transformation into cancer cells (see Schmidt S, et al. Oncoimmunology. 2020; 9(1):1809960; Dong Z, et al. Front Immunol. 2021; 11:586796). Therefore, HPV16-specific E7 and E6 can serve as immunogenic tumor-associated antigens. The methods described herein satisfy need of treating HPV16 infection and provide related advantages.

### SUMMARY OF DISCLOSURE

Provided herein are methods for treating cancer in a patient in need thereof. Such methods include administering to the patient an effective amount of engineered replication-competent tri-segmented arenavirus particles comprising duplicated (i.e. two) S-segments encoding a fusion protein of human papillomavirus strain 16 (HPV16) E7/E6. The effective amount of the arenavirus particle can be about 5×10⁵, about 5×10⁶, about 5×10⁷, about 1×10⁸, or about 5×10⁸ replication-competent virus focus-forming units (RCV FFU).

In some embodiments, the methods provided herein for treating cancer include treating an HPV 16⁺ cancer, regardless of origin. In some embodiments, the HPV 16⁺ cancer has been diagnosed as head and neck squamous cell carcinoma. In some embodiments, the HPV 16⁺ cancer has been diagnosed as anal cancer. In some embodiments, the HPV 16⁺ cancer has been diagnosed as cervical cancer. In some embodiments, the HPV 16⁺ cancer has been diagnosed as vaginal cancer. In some embodiments, the HPV 16⁺ cancer has been diagnosed as vulvar cancer.

In some embodiments, the patient had tumor progression or recurrence on at least one standard-of-care therapy prior to the method. In some specific embodiments, the at least one standard-of-care therapy comprises pembrolizumab monotherapy. In other embodiments, the patient has only target lesions in lymph nodes.

In some embodiments, the methods provided herein include administration of engineered replication-competent tri-segmented arenavirus particles using intravenous injection, intratumoral injection or a combination thereof. Accordingly, in some embodiments, administration of the engineered replication-competent tri-segmented arenavirus particle described herein includes intravenous injection. In some embodiments, administration of the engineered replication-competent tri-segmented arenavirus particle described herein includes intratumoral injection. In some embodiments, administration of the engineered replication-competent tri-segmented arenavirus particle described herein includes an intratumoral injection followed by an intravenous injection.

In some embodiments, the intravenous injections are administered with a frequency of every 2 weeks, every 3 weeks, every 4 weeks, every 5 weeks, every 6 weeks, every 7 weeks, or every 8 weeks.

In some embodiments, the intravenous injections are ongoing or are administered for a limited number of cycles. In specific embodiments, the limited number of cycles is two, three, four, five, or six. In other specific embodiments, the effective amount of the engineered replication-competent tri-segmented arenavirus particle administered for a limited number of cycles is one log order more than the effective amount used in the ongoing intravenous injections. In other embodiments, the intravenous injections are ongoing and are first administered with a higher frequency followed by a lower frequency. In specific embodiments, the intravenous injections are ongoing and are first administered with a frequency of every 3 weeks followed by a frequency of every 6 weeks. In one embodiment, the intravenous injections are ongoing and are first administered with a frequency of every 3 weeks for 4 cycles followed by a frequency of every 6 weeks for subsequent cycles. In other specific embodiments, the intravenous injections are ongoing and are first administered with a frequency of every 4 weeks followed by a frequency of every 8 weeks. In one embodiment, the intravenous injections are ongoing and are first administered with a frequency of every 4 weeks for 4 cycles followed by a frequency of every 8 weeks for subsequent cycles. In further embodiments, the administration of the engineered replication-competent tri-segmented arenavirus particle comprises intratumoral injections.

In some embodiments, the methods provided herein can also include administering an effective amount of an immune checkpoint inhibitor. An exemplary immune checkpoint inhibitor that is particularly useful for use in the methods described herein include an anti-PD-1 (programmed cell death protein 1) checkpoint inhibitor. Such an anti-PD-1 checkpoint inhibitor can be an antibody, such as nivolumab, pembrolizumab, pidilizumab or cemiplimab.

In some embodiments, the methods provided herein use engineered replication-competent tri-segmented arenavirus particles comprising Construct 1 as described herein. In some embodiments, the engineered replication-competent tri-segmented arenavirus particles are derived from LCMV, including the MP strain, WE strain, Armstrong strain, Armstrong Clone 13 strain or LCMV clone 13 strain expressing the glycoprotein of LCMV strain WE instead of endogenous LCMV clone 13 glycoprotein. In one embodiment, the effective amount of Construct 1 is about 5×10⁶ RCV FFU, and Construct 1 is administered with a frequency of every 3 weeks.

In some embodiments, the methods provided herein use engineered replication-competent tri-segmented arenavirus particles comprising Construct 2 as described herein. In some embodiments, the engineered replication-competent tri-segmented arenavirus particles are derived from Pichinde virus (PICV), including the strain Munchique CoAn4763 isolate P18, P2 strain, or any of the several isolates described by Trapido and colleagues (Trapido et al, 1971, Am J Trop Med Hyg, 20: 631-641).

In some embodiments, the methods provided herein results in a change in cytokine or chemokine levels in the serum of the patient as compared to the pre-treatment level of the patient. In some specific embodiments, the cytokines and chemokines comprise IFN-γ, IL-12p40, IL-15, IFN-inducible protein (IP)-10, and TNFα.

In some embodiments, the methods provided herein result in an increase of HPV16 E7/E6-specific T cells in the serum of the patient as compared to the pre-treatment level of the patient. In some specific embodiments, the HPV16 E7/E6-specific T cells are positive for CD8, IFN-γ, TNFα, and/or CD107a. In other embodiments, the T cells described above are detected without prior in-vitro stimulation and/or expansion. In still other embodiments, the method results in more T cells infiltrating into tumor tissues as compared to the pre-treatment level of the patient or patients receiving placebo.

In some embodiments, the method results in one or more improved efficacy endpoint using Response Evaluation Criteria in Solid Tumors (RECIST) and/or Immune Response Evaluation Criteria in Solid Tumors (iRECIST), compared to the pre-treatment level of the patient or patients receiving placebo. In some specific embodiments, the one or more improved efficacy endpoint comprises higher percentage of objective response rate, higher percentage of disease control rate, higher percentage of partial response, longer progression-free survival, and/or longer overall survival.

Also provided herein is a method for treating cancer in a patient in need thereof comprising: (i) administering to the patient an effective amount of engineered replication-competent tri-segmented arenavirus particles comprising two S-segments encoding a fusion protein of HPV16 E7/E6 derived from a first arenavirus species, and its effective amount is about 5×10⁵, 1×10⁶, 5×10⁶, 1×10⁷, 5×10⁷, 1×10⁸, 5×10⁸, or 1×10⁹ RCV FFU; and (ii) administering to the patient an effective amount of engineered replication-competent tri-segmented arenavirus particles comprising two S-segments encoding a fusion protein of HPV16 E7/E6 derived from a second arenavirus species, and its effective amount is about 5×10⁵, 1×10⁶, 5×10⁶, 1×10⁷, 5×10⁷, 1×10⁸, 5×10⁸, or 1×10⁹ RCV FFU.

In some embodiments, the method provided herein further comprises repeating (i) and/or (ii). In some embodiments, the arenavirus species in (i) is LCMV, and the arenavirus species in (ii) is PICV. In other embodiments, the arenavirus species in (i) is PICV, and the arenavirus species in (ii) is LCMV.

Provided herein is a method for treating cancer in a patient in need thereof comprising one or more session, wherein each session comprises: (i) administering to the patient an effective amount of engineered replication-competent tri-segmented arenavirus particles comprising two S-segments encoding a fusion protein of human papillomavirus strain 16 (HPV16) E7/E6 derived from a first arenavirus species, wherein the effective amount is about 5×10⁵, 1×10⁶, 5×10⁶, 1×10⁷, 5×10⁷, 1×10⁸, 5×10⁸, or 1×10⁹ replication-competent virus focus-forming units (RCV FFU); and (ii) administering to the patient an effective amount of engineered replication-competent tri-segmented arenavirus particles comprising two S-segments encoding a fusion protein of HPV16 E7/E6 derived from a second arenavirus species at a time point around half of the session, wherein the effective amount is about 5×10⁵, 1×10⁶, 5×10⁶, 1×10⁷, 5×10⁷, 1×10⁸, 5×10⁸, or 1×10⁹ RCV FFU. In some embodiments, the first arenavirus species in (i) is lymphocytic choriomeningitis virus (LCMV), and the second arenavirus species in (ii) is Pichinde virus (PICV). In other embodiments, the first arenavirus species in (i) is PICV, and the second arenavirus species in (ii) is LCMV.

In some embodiments, provided herein is a method comprising one or more session, wherein each session comprises: (i). administering to the patient an effective amount of Construct 2, wherein the effective amount is about 1×10⁶, 1×10⁷, 1×10⁸, or 1×10⁹ RCV FFU; and (ii). administering to the patient an effective amount of Construct 1 at a time point around half of the session, wherein the effective amount is about 5×10⁵, 5×10⁶, 5×10⁷, 1×10⁸, or 5×10⁸ RCV FFU. In some embodiments, the administration of the engineered replication-competent tri-segmented arenavirus particles in (i) and (ii) comprises intravenous injection. In some specific embodiments, each session lasts for 4 weeks, 6 weeks, 8 weeks, 10 weeks, 12 weeks, 14 weeks, or 16 weeks.

In some embodiments, the methods provided herein for treating cancer include treating an HPV 16⁺ cancer, regardless of origin. In some embodiments, the HPV 16⁺ cancer has been diagnosed as head and neck squamous cell carcinoma. In some embodiments, the HPV 16+ cancer has been diagnosed as anal cancer. In some embodiments, the HPV 16⁺ cancer has been diagnosed as cervical cancer. In some embodiments, the HPV 16⁺ cancer has been diagnosed as vaginal cancer.

In some embodiments, the patient had tumor progression or recurrence on at least one standard-of-care therapy prior to the method. In some specific embodiments, the at least one standard-of-care therapy comprises pembrolizumab monotherapy. In other embodiments, the patient has only target lesions in lymph nodes.

In some embodiments, the methods provided herein include administration of engineered replication-competent tri-segmented arenavirus particles using intravenous injection, intratumoral injection or a combination thereof. Accordingly, in some embodiments, administration of the engineered replication-competent tri-segmented arenavirus particle described herein includes intravenous injection. In some embodiments, administration of the engineered replication-competent tri-segmented arenavirus particle described herein includes intratumoral injection. In some embodiments, administration of the engineered replication-competent tri-segmented arenavirus particle described herein includes an intratumoral injection followed by an intravenous injection.

In some embodiments, the sessions are ongoing or are repeated for a limited number of sessions. In some specific embodiments, the limited number of sessions is two, three, four, five, or six. In some specific embodiments, the effective amount of the engineered replication-competent tri-segmented arenavirus particles administered for a limited number of sessions is one log order more than the effective amount used in the ongoing sessions. In some specific embodiments, the intravenous injections are ongoing and are first administered in shorter sessions followed by longer sessions. In some particular embodiments, the intravenous injections are ongoing and are first administered with sessions each lasting 6 weeks followed by sessions each lasting 12 weeks. In one embodiment, the intravenous injections are ongoing and are first administered with 2 sessions each lasting 6 weeks followed by sessions each lasting 12 weeks. In some particular embodiments, the intravenous injections are ongoing and are first administered with sessions each lasting 8 weeks followed by sessions each lasting 16 weeks. In one embodiment, the intravenous injections are ongoing and are first administered with 2 sessions each lasting 8 weeks followed by sessions each lasting 16 weeks.

In some embodiments, the method further comprises an intratumoral injection prior to the intravenous injections. In specific embodiments, the intratumoral injection is administered 3 weeks prior to the intravenous injections. In yet specific embodiments, the intratumoral injection is administered with Construct 1.

In some embodiments, the methods provided herein can also include administering an effective amount of an immune checkpoint inhibitor. An exemplary immune checkpoint inhibitor that is particularly useful for use in the methods described herein include an anti-PD-1 (programmed cell death protein 1) checkpoint inhibitor. Such an anti-PD-1 checkpoint inhibitor can be an antibody, such as nivolumab, pembrolizumab, pidilizumab or cemiplimab.

In some embodiments, the methods provided herein use engineered replication-competent tri-segmented arenavirus particles comprising Construct 1 as described herein. In some embodiments, the engineered replication-competent tri-segmented arenavirus particles are derived from LCMV, including the MP strain, WE strain Armstrong strain, Armstrong Clone 13 strain or LCMV clone 13 strain expressing the glycoprotein of LCMV strain WE instead of endogenous LCMV clone 13 glycoprotein.

In some embodiments, the methods provided herein use engineered replication-competent tri-segmented arenavirus particles comprising Construct 2 as described herein. In some embodiments, the engineered replication-competent tri-segmented arenavirus particles are derived from PICV, including the strain Munchique CoAn4763 isolate P18, P2 strain, or any of the several isolates described by Trapido and colleagues (Trapido et al, 1971, Am J Trop Med Hyg, 20: 631-641).

In some embodiments, the methods provided herein results in a change in the levels of cytokine or chemokine levels in the serum of the patient as compared to the pre-treatment level of the patient. In some specific embodiments, the cytokines and chemokines comprise IFN-γ, IL-12p40, IL-15, IFN-inducible protein (IP)-10, and TNFα.

In some embodiments, the methods provided herein results in an increase of HPV16 E7/E6-specific T cells in the serum of the patient as compared to the pre-treatment level of the patient. In some specific embodiments, the HPV16 E7/E6-specific T cells are positive for CD8, IFN-γ, TNFα, and/or CD107a. In other embodiments, the T cells described above are detected without prior in-vitro stimulation and/or expansion. In still other embodiments, the method results in more T cells infiltrating into tumor tissues as compared to the pre-treatment level of the patient or patients receiving placebo.

In some embodiments, the method results in one or more improved efficacy endpoint using Response Evaluation Criteria in Solid Tumors (RECIST) and/or Immune Response Evaluation Criteria in Solid Tumors (iRECIST), compared to the pre-treatment level of the patient or patients receiving placebo. In some specific embodiments, the one or more improved efficacy endpoint comprises higher percentage of objective response rate, higher percentage of disease control rate, higher percentage of partial response, longer progression-free survival, and/or longer overall survival.

Provided herein is a method for treating cancer in a patient in need thereof comprising: (i) administering to the patient an effective amount of Construct 1, wherein the effective amount is about 5×10⁵, 5×10⁶, 5×10⁷, 1×10⁸, or 5×10⁸ replication-competent virus focus-forming units (RCV FFU), and wherein Construct 1 is administered intravenously with a frequency of every 3 weeks for 4 cycles followed by ongoing cycles with a frequency of every 6 weeks; and administering to the patient 200 mg of pembrolizumab intravenously with a frequency of every 3 weeks or 400 mg of pembrolizumab intravenously with a frequency of every 6 weeks.

Provided herein is a method for treating cancer in a patient in need thereof comprising multiple sessions, wherein each session comprises: i. administering to the patient an effective amount of Construct 2 intravenously, wherein the effective amount is about 1×10⁶ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks.

Provided herein is a method for treating cancer in a patient in need thereof comprising multiple sessions, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks.

Provided herein is a method for treating cancer in a patient in need thereof comprising multiple sessions, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks.

Provided herein is a method for treating cancer in a patient in need thereof comprising multiple sessions, wherein each session comprises: administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks.

Provided herein is a method for treating cancer in a patient in need thereof comprising multiple sessions, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 1×10⁸ RCV FFU, and wherein the first two sessions each lasts for 6 weeks, and the following ongoing sessions each lasts for 12 weeks.

Provided herein is a method for treating cancer in a patient in need thereof comprising multiple sessions, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁸ RCV FFU, and wherein the first two sessions each lasts for 6 weeks, and the following ongoing sessions each lasts for 12 weeks.

Provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁶ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 1×10⁸ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁸ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Provided herein is a method for treating cancer in a patient in need thereof comprising (1) administering intratumorally to the patient an effective amount of Construct 1, wherein the effective amount of Construct 1 is about 5×10⁶ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁶ RCV FFU; and ii administering intravenously to the patient an effective amount of Construct 1 at a time point around half of the session, wherein the effective amount is about 5×10⁶ RCV FFU, and wherein the first two sessions each lasts for 6 weeks, and the following ongoing sessions each lasts for 12 weeks.

Provided herein is a method for treating cancer in a patient in need thereof comprising (1) administering intratumorally to the patient an effective amount of Construct 1, wherein the effective amount of Construct 1 is about 5×10⁶ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁷ RCV FFU; and ii. administering intravenously to the patient an effective amount of Construct 1 at a time point around half of the session, wherein the effective amount is about 5×10⁶ RCV FFU, and wherein the first two sessions each lasts for 6 weeks, and the following ongoing sessions each lasts for 12 weeks.

Provided herein is a method for treating cancer in a patient in need thereof comprising (1) administering intratumorally to the patient an effective amount of Construct 1, wherein the effective amount of Construct 1 is about 5×10⁷ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁷ RCV FFU; and ii administering intravenously to the patient an effective amount of Construct 1 at a time point around half of the session, wherein the effective amount is about 5×10⁷ RCV FFU, and wherein the first two sessions each lasts for 6 weeks, and the following ongoing sessions each lasts for 12 weeks.

Provided herein is a method for treating cancer in a patient in need thereof comprising (1) administering intratumorally to the patient an effective amount of Construct 1, wherein the effective amount of Construct 1 is about 5×10⁷ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁸ RCV FFU; and ii administering intravenously to the patient an effective amount of Construct 1 at a time point around half of the session, wherein the effective amount is about 5×10⁷ RCV FFU, and wherein the first two sessions each lasts for 6 weeks, and the following ongoing sessions each lasts for 12 weeks.

Provided herein is a method for treating cancer in a patient in need thereof comprising (1) administering intratumorally to the patient an effective amount of Construct 1, wherein the effective amount of Construct 1 is about 1×10⁸ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁸ RCV FFU; and ii administering intravenously to the patient an effective amount of Construct 1 at a time point around half of the session, wherein the effective amount is about 1×10⁸ RCV FFU, and wherein the first two sessions each lasts for 6 weeks, and the following ongoing sessions each lasts for 12 weeks.

Provided herein is a method for treating cancer in a patient in need thereof comprising (1) administering intratumorally to the patient an effective amount of Construct 1, wherein the effective amount of Construct 1 is about 5×10⁸ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁸ RCV FFU; and ii administering intravenously to the patient an effective amount of Construct 1 at a time point around half of the session, wherein the effective amount is about 5×10⁸ RCV FFU, and wherein the first two sessions each lasts for 6 weeks, and the following ongoing sessions each lasts for 12 weeks.

Provided herein is a method for treating cancer in a patient in need thereof comprising administering to the patient an effective amount of Construct 1, wherein the effective amount is about 5×10⁶, 5×10⁷, 5×10⁸, 1×10⁹, or 5×10⁹ replication-competent virus focus-forming units (RCV FFU), and wherein Construct 1 is administered intravenously with a frequency of every 3 weeks for 3 cycles and the method ends after 3 cycles.

Provided herein is a method for treating cancer in a patient in need thereof comprising 3 sessions, wherein each session comprises i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁷ RCV FFU, and wherein each sessions lasts for 6 weeks, and the method ends after 3 sessions.

Provided herein is a method for treating cancer in a patient in need thereof comprising 3 sessions, wherein each session comprises i. administering to the patient an effective amount of Construct 2 intravenously, wherein the effective amount is about 1×10⁸ replication-competent virus focus-forming units (RCV FFU); and administering to the patient an effective amount of Construct 1 intravenously at a time point around half of the session, wherein the effective amount is about 5×10⁷ RCV FFU, and wherein each sessions lasts for 6 weeks, and the method ends after 3 sessions.

Provided herein is a method for treating cancer in a patient in need thereof comprising 3 sessions, wherein each session comprises i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁸ RCV FFU, and wherein each sessions lasts for 6 weeks, and the method ends after 3 sessions.

Provided herein is a method for treating cancer in a patient in need thereof comprising 3 sessions, wherein each session comprises i. administering to the patient an effective amount of Construct 2 intravenously, wherein the effective amount is about 1×10⁹ replication-competent virus focus-forming units (RCV FFU); and administering to the patient an effective amount of Construct 1 intravenously at a time point around half of the session, wherein the effective amount is about 5×10⁸ RCV FFU, and wherein each sessions lasts for 6 weeks, and the method ends after 3 sessions.

Provided herein is a method for treating cancer in a patient in need thereof comprising 3 sessions, wherein each session comprises i. administering to the patient an effective amount of Construct 2 intravenously, wherein the effective amount is about 1×10⁹ replication-competent virus focus-forming units (RCV FFU); and ii. administering to the patient an effective amount of Construct 1 intravenously at a time point around half of the session, wherein the effective amount is about 1×10⁹ RCV FFU, and wherein each sessions lasts for 6 weeks, and the method ends after 3 sessions.

Provided herein is a method for treating cancer in a patient in need thereof comprising 3 sessions, wherein each session comprises i. administering to the patient an effective amount of Construct 2 intravenously, wherein the effective amount is about 1×10⁹ replication-competent virus focus-forming units (RCV FFU); ii. administering to the patient an effective amount of Construct 1 intravenously at a time point around half of the session, wherein the effective amount is about 5×10⁹ RCV FFU, and wherein each sessions lasts for 6 weeks, and the method ends after 3 sessions.

Provided herein is a nucleic acid comprising the nucleotide sequence of SEQ ID NOs: 1 or 2.

Also provided herein is a nucleic acid comprising the nucleotide sequence of SEQ ID NOs: 3, 4, 5, 6, 7, or 8.

In some embodiments, the nucleic acid provided herein is RNA.

Provided herein is a host cell comprising a nucleotide sequence of SEQ ID NOs: 1, 2, 3, 4, 5, 6, 7, or 8.

Provided herein is a tri-segmented LCMV particle comprising the nucleotide sequences of SEQ ID NOs: 3, 4, and 5.

Also provided herein is a tri-segmented Pichinde virus particle comprising the nucleotide sequences of SEQ ID NOs: 6, 7, and 8.

Provided herein is a pharmaceutical composition comprising a tri-segmented LCMV particle comprising the nucleotide sequences of SEQ ID NOs: 3, 4, and 5, or a tri-segmented Pichinde virus particle comprising the nucleotide sequences of SEQ ID NOs: 6, 7, and 8 and a pharmaceutically acceptable carrier.

In some embodiments, the tri-segmented arenavirus particle comprising the dinucleotide optimized HPV16 E7E6 nucleotide sequence can have stable expression of the HPV antigen after being passaged at least 4, 5, 6, 7, 8, 9, or 10 generations, can have consistent expression of the encoded HPV fusion protein or induce strong immune responses against the encoded HPV fusion protein.

### BRIEF DESCRIPTION OF THE DRAWINGS

FIG. 1 shows HPV cancer burden and cancers related to HPV.

FIGS. 2A to 2C show schematic depictions of a wild-type arenavirus (e.g., LCMV or PICV), a replication-competent tri-segmented arenavirus particle derived from the arenavirus LCMV or PICV, encoding a non-oncogenic fusion protein of HPV16⁺ E7/E6, and the mode of attenuation of replicating tri-segmented arenavirus particles. FIG. 2A shows arenavirus (LCMV and PICV) wild-type particle (left) and its genome (right). The antisense RNA genome encodes 4 viral proteins: GP (glycoprotein), NP (nucleoprotein), L (RNA-directed RNA polymerase), and Z (RING finger protein Z). FIG. 2B shows an engineered tri-segmented arenavirus particle (Construct 1; LCMV-based vector, Construct 2: PICV-based vector) that contains artificially duplicated S-segments encoding a fusion protein of HPV16 E7/E6 with 5 amino acid mutations to abrogate the oncogenic potential of E7 and E6 as indicated by asterisks (*) and either GP or NP, as well as an L-segment. FIG. 2C shows inefficient packaging of the 3 genome segments results in attenuation of Construct 1 and Construct 2 compared to the wild-type LCMV and PICV, respectively.

FIGS. 3A to 3C show results of pre-clinical studies of the engineered LCMV-based tri-segmented arenavirus particle of FIG. 2B in mice. Such particles target dendritic cells and other antigen presenting cells for stimulation of the immune system in an antigen specific manner. The engineered LCMV-based tri-segmented arenavirus particles induce a potent T cell response directed specifically against HPV 16⁺ tumor cells. FIG. 3A shows immunogenicity of the engineered LCMV-based tri-segmented arenavirus particles (Construct 1) illustrated by percentages of blood HPV16 E7-specific CD8 (CD8⁺B220⁻) T cells in healthy mice immunized with increasing doses of the particles by intravenous (IV) administration. FIG. 3B shows kinetics of tumor growth in HPV16⁺ TC-1 tumor-bearing mice treated with increasing doses of the engineered LCMV-based tri-segmented arenavirus particles following IV administration. FIG. 3C shows survival curves of HPV16⁺ TC-1 tumor-bearing mice treated with the engineered LCMV-based tri-segmented arenavirus particles (Construct 1) following intratumoral (IT) or IV administration.

FIG. 4 shows the treatment study design, which includes both a dose escalation and dose expansion strategy. Construct 1=the engineered LCMV-based tri-segmented arenavirus particle of FIG. 2B; RP2D=Recommended Phase II Dose; HNSCC=Head and Neck Squamous Cell Carcinoma; HPV 16=Human Papillomavirus 16; IV=Intravenous; IT=Intratumoral; RCV FFU=replication-competent virus focus-forming units; anti-PD-1=PD-1 immune checkpoint inhibitor.

FIG. 5 shows the treatment design for a phase 1 dose-escalation study.

FIGS. 6A to 6B show results of distinct serum cytokine or chemokine signatures after treatment with Construct 1. FIG. 6A shows results of 30-plex cytokine and chemokine analyses for twelve patients over eight time points. Day 4 data were available for ten of the twelve patients. Analytes (pg/mL) were converted to z scores. Hierarchical clustering was performed by visit day and each analyte level. FIG. 6B shows effects of treatment with Construct 1 on expression of select key cytokines 4 days post-treatment. Nine of twelve patients had both samples from baseline and day 4.

FIGS. 7A to 7F show results of circulating HPV E6/E7-specific poly-functional T cells after single administration of Construct 1 or Construct 2. FIG. 7A shows a direct IFN-γ ELISpot analysis of changes in spot-forming units from baseline to day 15 after administration of a single IV dose of Construct 1 or Construct 2. FIG. 7B shows increase in E6/E7-specific T cells in patients treated with a single IV dose of Construct 1 or Construct 2. FIG. 7C shows the frequency of CD4⁺ and CD8⁺ among total peripheral T cell population and the frequency of IFN-γ⁺, TNF-α⁺, and CD107a⁺ after being gated on CD8⁺ T cells from one patient who received one dose of Construct 1. FIG. 7D shows the frequency of CD4⁺ and CD8⁺ among total peripheral T cell population and the frequency of IFN-γ⁺, TNF-α⁺, and CD107a⁺ after being gated on CD8⁺ T cells from another patient who received one dose of Construct 1. FIG. 7E shows the frequency of CD4⁺ and CD8⁺ among total peripheral T cell population and the frequency of IFN-γ⁺, TNF-α⁺, and CD107a⁺ after being gated on CD8 T cells from one patient who received one dose of Construct 2. FIG. 7F shows pie charts from each of the three patients, which represents the relative frequency of HPV16 E6/E7-specific CD8⁺ T cells in combination of the three functional response markers (i.e., CD107a, IFN-γ, TNF-α) after a single administration of Construct 1 or Construct 2.

FIG. 8 shows the E7E6-NP-S-segment 1 (2648 bp) of Construct 1 as per FIG. 2B. The following elements are indicated from 5′ to 3′ of the disclosed sequence. The 5′ untranslated region (UTR) based on LCMV cl13 S-segment (1-78 bp) is shown without highlight; the synthetic fusion protein consisting of Human Papilloma Virus type 16 (HPV 16) proteins E6 and E7 (79-846 bp) is shown in green; the intergenic region (IGR) based on LCMV cl13 S-segment (847-910 bp) is shown in blue; the nucleoprotein (NP) based on LCMV cl13 (911-2587 bp) is shown in gray; the 3′ untranslated region (UTR) based on LCMV cl13 S-segment (2588-2648 bp) is shown without highlight.

FIG. 9 shows the E7E6-GP-S-segment 2 (2648 bp) of Construct 1 as per FIG. 2B. The following elements are indicated from 5′ to 3′ of the disclosed sequence. The 5′ untranslated region (UTR) based on LCMV cl13 S-segment (1-78 bp) is shown without highlight; the synthetic fusion protein consisting of Human Papilloma Virus type 16 (HPV 16) proteins E6 and E7 (79-846 bp) is shown in green; the intergenic region (IGR) based on LCMV cl13 S-segment (847-910 bp) is shown in blue; the glycoprotein (GP) based on LCMV WE (911-2407 bp) is shown in yellow; the 3′ untranslated region (UTR) based on LCMV cl13 S-segment (2408-2468 bp) is shown without highlight.

FIG. 10 shows the L-segment (7229 bp) of Construct 1 as per FIG. 2B. The following elements are indicated from 5′ to 3′ of the disclosed sequence. The 5′ untranslated region (UTR) based on LCMV cl13 L-segment (1-89 bp) is shown without highlight; the matrix protein (Z) based on LCMV cl13 (90-362 bp) is shown in green; the intergenic region (IGR) based on LCMV cl13 L-segment (363-564 bp) is shown in blue; the ribonucleic acid dependent ribonucleic acid polymerase protein (L) based on LCMV cl13 (565-7197 bp) is shown in gray; the 3′ untranslated region (UTR) based on LCMV cl13 L-segment (7198-7229 bp) is shown without highlight.

FIG. 11 shows the E7E6-NP-S-segment 1 (2663 bp) of Construct 2 as per FIG. 2B. The following elements are indicated from 5′ to 3′ of the disclosed sequence. The 5′ untranslated region (UTR) based on PICV p18 S-segment (1-52 bp) is shown without highlight; the synthetic fusion protein consisting of Human Papilloma Virus type 16 (HPV 16) proteins E6 and E7 (53-820 bp) is shown in green; the intergenic region (IGR) based on PICV p18 S-segment (821-894 bp) is shown in blue; the nucleoprotein (NP) based on PICV p18 (895-2580 bp) is shown in gray; the 3′ untranslated region (UTR) based on PICV p18 S-segment (2581-2663 bp) is shown without highlight.

FIG. 12 shows the E7E6-GP-S-segment 2 (2504 bp) of Construct 2 as per FIG. FIG. 2B. The following elements are indicated from 5′ to 3′ of the disclosed sequence. The 5′ untranslated region (UTR) based on PICV p18 S-segment (1-52 bp) is shown without highlight; the synthetic fusion protein consisting of Human Papilloma Virus type 16 (HPV 16) proteins E6 and E7 (53-820 bp) is shown in green; the intergenic region (IGR) based on PICV p18 S-segment (821-894 bp) is shown in blue; the glycoprotein (GP) based on PICV p18 (895-2421 bp) is shown in yellow; the 3′ untranslated region (UTR) based on PICV p18 S-segment (2422-2504 bp) is shown without highlight.

FIG. 13 shows the L-segment (7058 bp) of Construct 2 as per FIG. 2B. The following elements are indicated from 5′ to 3′ of the disclosed sequence. The 5′ untranslated region (UTR) based on PICV p18 L-segment (1-85 bp) is shown without highlight; the matrix protein (Z) based on PICV p18 (86-373 bp) is shown in red; the intergenic region (IGR) based on PICV p18 L-segment (374-443 bp) is shown in blue; the ribonucleic acid dependent ribonucleic acid polymerase protein (L) based on PICV p18 (444-7028 bp) is shown in gray; the 3′ untranslated region (UTR) based on PICV p18 L-segment (7029-7058 bp) is shown without highlight.

FIGS. 14A to 14E show efficacy of Construct 1 and/or Construct 2 in a mice model bearing HPV16⁺ tumors. FIG. 14A shows the correlation between the dose of Construct 1 and percentage of HPV16 E7-specific CD8⁺ B220⁻ T cells. CD8=cluster of differentiation 8, E7=antigenic E7 fusion protein from human papillomavirus 16. FIG. 14B shows the changes of tumor volume over time in response to treatment with different doses of Construct 1; FFU=focus-forming units, HPV=human papilloma virus, RCV=replication-competent virus. FIG. 14C shows the changes of tumor volume over time in response to treatment with Construct 1 or an arenaviral vector expressing a non-relevant control antigen GFP via different administration routes (i.v.=intravenous; i.t.=intratumoral) in comparison to control animals treated with buffer only. E7E6=antigenic E7 and E6 fusion protein from human papillomavirus 16, G=group, GFP=green florescent protein, i.t. (IT)=intratumoral, i.v. (IV)=intravenous(ly), n=number of mice in each experimental group. FIG. 14D shows the change of percentage of HPV16 E7-specific CD8⁺B220⁻ T cells over time in mice in response to the indicated dosing regimens of Construct 1 and Construct 2 after i.v. administration of 10⁵ RCV FFU of each vector with a 21-day interval. CD=cluster of differentiation, E7E6=antigenic E7 and E6 fusion protein from human papillomavirus 16, G=group, dashes indicate the sequence of vector administration (prime/1st dose−boost/2nd dose). FIG. 14E shows the changes of tumor volume over time in response to the indicated dosing regimens of Construct 1 and Construct 2 after i.v. administration of 10⁵ RCV FFU of each vector with a 4, 7 or 10-day interval. G=group, d=days between prime and boost administration, dashes indicate the sequence of vector administration (prime/1st dose−boost/2nd dose).

FIG. 15 shows the experiment design in Example III, which includes phase I dose escalation and phase II dose expansion. Alt.=alternating, Approx=approximately, HNSCC=head and neck squamous cell carcinoma, HPV=human papilloma virus, IT=intratumoral, IV=intravenous(ly), n=number of patients, RP2D=recommended Phase II dose.

FIG. 16 shows the experiment design for backfill cohorts in Example III.

FIGS. 17A to 17I show efficacy data of Construct 1 monotherapy and Construct 2/Construct 1 alternating 2-vector therapy. FIG. 17A shows treatment duration in individual patients. Time on treatment=Last treatment/death date−first dose date+1; Pembrolizumab was added to the arenaviral vector therapy in 3 patients by investigators. EDC data was used for some patients due to missing/incorrect data entry on TLF as of the data transfer date. DL, dose level; EDC, electronic data capture; HNSCC, head and neck squamous cell carcinoma; IT, intratumoral; IV, intravenous; PR, partial response; Q2W, every 2 weeks; Q3W, every 3 weeks.

FIG. 17B shows target lesion (TL) sum of diameter (SOD) change from baseline of each individual patient. Striped areas indicate decrease in target lesion change after pembrolizumab was added to therapy. #Progression with non-evaluable scans, artificially assigned 2%. Non-oropharynx patients: A, anal; C, cervical; NP, nasopharynx; V, vaginal. IT, intratumoral; IV, intravenous; SOD, sum of diameters; TL, target lesion. Q2W=every two weeks; Q3W=every three weeks; DL=dose level. FIG. 17C shows the best target lesion (TL) sum of diameter (SOD) change from baseline by schedule/route of administration. Q2W=every two weeks; Q3W=every three weeks; DL=dose level; IV=intravenous; IT=intratumoral. FIG. 17D shows target lesion (TL) sum of diameter (SOD) change from baseline of each individual patient in a spider plot. Open squares represent scans performed after addition of pembrolizumab. One patient with non-evaluable efficacy scans is not shown on the spider plot. Target lesion with 60% decrease is lymph node measured <10 mm, therefore unconfirmed complete response. EDC data was used for some patients due to missing/incorrect data entry on TLF as of the data transfer date. EDC, electronic data capture; HNSCC, head and neck squamous cell carcinoma; SOD, sum of diameters; TL, target lesion; uCR, unconfirmed complete response. FIG. 17E shows progression-free survival (PFS) in treated patients. For patients who received pembrolizumab, PFS includes time after pembrolizumab had been added prior to RECIST progression. FIG. 17F shows target lesion change from baseline in patients with only lymph node lesions as well as in patients having only non-lymph node lesions or both, lymph node lesions as well as non-lymph node lesions. FIG. 17G shows Sum of diameter changes for RECIST evaluable lesions in patients receiving IV administration of Construct 1 dose level 2 every three weeks and IV administration of Construct 2/Construct 1 alternating 2-vector therapy. FIG. 17H shows a correlation between best sum of diameter change and time on treatment. FIG. 17I shows efficacy scans at baseline and subsequent time points during treatment.

FIG. 18A shows results of 30-plex cytokine and chemokine analyses after treatment with Construct 1 over nine time points. Day 4 data were available for ten of the twelve patients. Analytes (pg/mL) were converted to z scores. Hierarchical clustering was performed by visit day and each analyte level. FIG. 18B shows effects of treatment with Construct 1 monotherapy (DL 1 (5×10⁵ RCV FFU) or DL2 (5×10⁶ RCV)) or Construct 2/Construct 1 alternating 2-vector therapy (Construct 2: 1×10⁶ RCV FFU, Construct 1: 5×10⁶ RCV FFU) on expression of select key cytokines 4 days post-treatment.

FIGS. 19A to 19I show strong immunogenicity induced by Construct 1 monotherapy and Construct 2/Construct 1 alternating 2-vector therapy. FIG. 19A shows ELISpot result for six patients who received Construct 1 IV at DL2 (5×10⁶ RCV FFU) every 3 weeks. Thawed peripheral blood mononuclear cells (PBMCs) from all patients were stimulated with overlapping HPV16 E6/E7 peptides for 24 h (±2 h) for direct ex vivo IFN-γ ELISpot measurement. Shown is the number of spot forming units/1×10⁶ PBMCs. FIG. 19B shows ICS result for six patients who received Construct 1 IV at DL2 (5×10⁶ RCV FFU) every 3 weeks. PBMCs were stimulated for 6 hours with overlapping HPV16 E6/E7 peptides, washed for subsequent immunostaining for IFN-γ, TNF-α, IL-2 and CD107a, and analyzed by polychromatic flow cytometry. Shown is the percentage of IFN-γ⁺ cells among CD8 T cells. FIG. 19C shows ELISpot result for three patients who received Construct 2 at DL1 (1×10⁶ RCV FFU) and Construct 1 at DL2 (5×10⁶ RCV FFU) IV every 3 weeks. Thawed peripheral blood mononuclear cells (PBMCs) from all patients were stimulated with overlapping HPV16 E6/E7 peptides for 24 h (±2 h) for direct ex vivo IFN-γ ELISpot measurement. Shown is the number of spot forming units/1×10⁶ PBMCs. The upper limit of quantification (ULOQ) is indicated by a horizontal, dotted line. FIG. 19D shows ICS result for three patients who received Construct 2 at DL1 (1×10⁶ RCV FFU) and Construct 1 at DL2(5×10⁶ RCV FFU) IV every 3 weeks. PBMCs were stimulated for 6 hours with overlapping HPV16 E6/E7 peptides, washed for subsequent immunostaining for IFN-γ, TNF-α, IL-2 and CD107a, and analyzed by polychromatic flow cytometry. Shown is the percentage of IFN-γ⁺ cells among CD8 T cells. FIG. 19E shows flow cytometry results of CD8⁺ and CD4⁺ T cells over time in one patient receiving Construct 2/Construct 1 alternating 2-vector therapy. PBMCs were stimulated for 6 hours with E6/E7 peptides, washed for subsequent immunostaining and analyzed by polychromatic flow cytometry. Cells were gated on CD3+ cells and numbers above CD8 gates show the percentage of CD8+ cells among CD3+ T cells. FIG. 19F shows flow cytometry results of T cells that express IFN-γ, TNFα, or CD107a over time in one patient receiving Construct 2/Construct 1 alternating 2-vector therapy. PBMCs were stimulated for 6 hours with overlapping E6/E7 peptide pool, washed for subsequent immunostaining for IFN-γ, TNF-α, IL-2 and CD107a, and analyzed by polychromatic flow cytometry. Cells were gated on CD3+CD8+ T cells. FIG. 19G to 19I show changes in PBMCs from baseline to the maximal response (Max) for individual patients. Max refers to the highest E6/E7 specific T cell responses measured by IFN-γ ELISpot for each individual patient. FIG. 19G shows the change of white blood cell counts (WBC) from baseline to Max as WBC 10³/μl blood after >2 arenaviral vector administrations (left panel). The right panel shows the change of the CD8/CD4 ratio from baseline to Max calculated from flow cytometric analyses after CD19, NK1.1, CD3, CD8 and CD4 staining of blood samples. FIG. 19H shows IFN-γ ELISpot results for baseline and Max as spot forming units/10⁶ PBMCs. FIG. 19I are representative pseudo color plots from PBMC samples after intracellular cytokine staining (see FIGS. 19B, D and F). Left plots are from baseline, right plots are from Max. The upper row is from a patient treated with Construct 1 DL2 (5×10⁶ RCV FFU) IV-IV, the lower row is from a patient treated with Construct 2 DL1 (1×10⁶ RCV FFU) and Construct 1 DL2 (5×10⁶ RCV FFU) alternating 2-vector therapy.

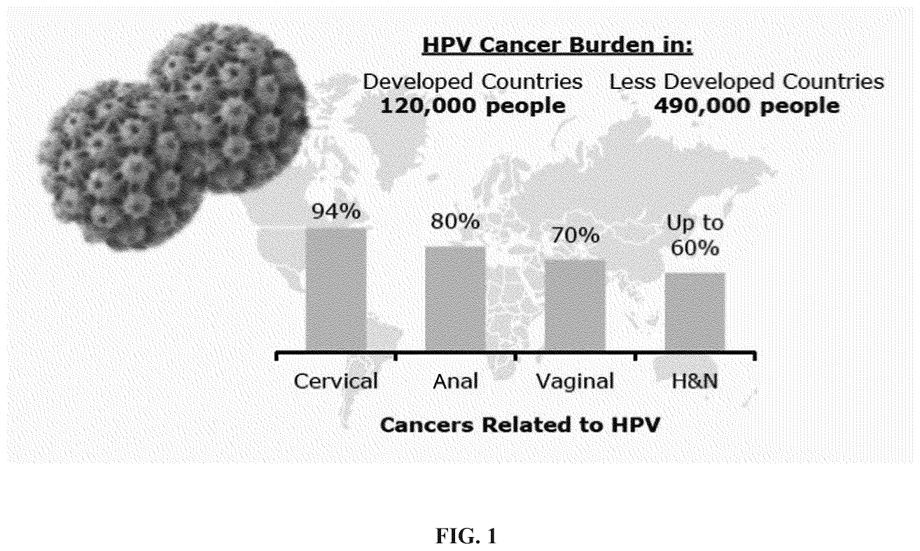

### DETAILED DESCRIPTION OF THE DISCLOSURE

As used herein, the term “cycle,” when used in methods for treating cancer with one species of engineered replication-competent tri-segmented arenavirus particles, is intended to refer to an administration day and the days before the next administration.

As used herein, the term “session,” when used in methods for treating cancer with two species of engineered replication-competent tri-segmented arenavirus particles in an alternating 2-vector therapeutic approach, is intended to refer to an administration day of the first species, the days before an administration of the second species, an administration of the second species, and the days before another administration of the first species.

Provided herein are methods for treating cancer in a patient in need thereof. Such methods include administering to the patient an effective amount of engineered replication-competent tri-segmented arenavirus particles having duplicated (i.e. two) S-segments encoding a fusion protein of HPV16 E7/E6. The effective amount of the particles can be about 5×10⁵, about 5×10⁶, about 5×10⁷ RCV FFU, about 1×10⁸ RCV FFU, or about 5×10⁸ RCV FFU. Accordingly, in some embodiments, provided herein is a method for treating cancer in a patient in need thereof that includes administering to the patient an effective amount of engineered replication-competent tri-segmented arenavirus particles having duplicated (i.e. two) S-segments encoding a fusion protein of HPV16 E7/E6, wherein the effective amount is about 5×10⁵ RCV FFU. In some embodiments, provided herein is a method for treating cancer in a patient in need thereof that includes administering to the patient an effective amount of engineered replication-competent tri-segmented arenavirus particles having duplicated (i.e. two) S-segments encoding a fusion protein of HPV16 E7/E6, wherein the effective amount is about 5×10⁶ RCV FFU. In some embodiments, provided herein is a method for treating cancer in a patient in need thereof that includes administering to the patient an effective amount of engineered replication-competent tri-segmented arenavirus particles having duplicated (i.e. two) S-segments encoding a fusion protein of human papillomavirus strain 16 (HPV16) E7/E6, wherein the effective amount is about 5×10⁷ RCV FFU. In some embodiments, provided herein is a method for treating cancer in a patient in need thereof that includes administering to the patient an effective amount of engineered replication-competent tri-segmented arenavirus particles having duplicated (i.e. two) S-segments encoding a fusion protein of human papillomavirus strain 16 (HPV16) E7/E6, wherein the effective amount is about 5×10⁸ RCV FFU. In some embodiments, provided herein is a method for treating cancer in a patient in need thereof that includes administering to the patient an effective amount of engineered replication-competent tri-segmented arenavirus particles having duplicated (i.e. two) S-segments encoding a fusion protein of HPV16 E7/E6, wherein the effective amount is about 1×10⁸ RCV FFU.

Methods for generating engineered replication-competent tri-segmented arenavirus particles for use in the methods described herein are well known in the art. Exemplary methods can be found in US Patent Application Publication US-2017-0319673-A1, published Nov. 9, 2017, US Patent Application Publication US-2019-0135875-A1, published May 9, 2019, and US Patent Application Publication US-2018-0179257-A1, published Jun. 28, 2018, which are each incorporated herein by reference. In view of these publications, a person of skill in the art would understand that such engineered replication-competent tri-segmented arenavirus particles having duplicated (i.e. two) S-segments as described herein include replication-competent tri-segmented arenavirus particles wherein the open reading frame (ORF) encoding the NP protein is present on one S-segment, while the ORF encoding the GP protein is present on the other S-segment. Also provided in these publications are descriptions of pharmaceutical compositions having engineered replication-competent tri-segmented arenavirus particles that can be used in the methods described herein.

In some embodiments, the methods provided herein for treating cancer include treating any HPV 16⁺ cancer, regardless of origin. In some embodiments, the HPV 16⁺ cancer has been diagnosed as head and neck squamous cell carcinoma. In some embodiments, the HPV 16⁺ cancer has been diagnosed as anal cancer. In some embodiments, the HPV 16⁺ cancer has been diagnosed as cervical cancer. In some embodiments, the HPV 16⁺ cancer has been diagnosed as vaginal cancer. In some embodiments, the HPV 16⁺ cancer has been diagnosed as vulvar cancer. Accordingly, in some embodiments, the methods provided herein are for treating HPV 16⁺ cancer (e.g., head and neck squamous cell carcinoma, cervical cancer, anal cancer, vulvar, or vaginal cancer) in a patient in need thereof by administering to the patient an effective amount of engineered replication-competent tri-segmented arenavirus particles having duplicated (i.e. two) S-segments encoding a fusion protein of HPV16 E7/E6, wherein the effective amount is about 5×10⁵ RCV FFU, about 5×10⁶ RCV FFU, about 5×10⁷ RCV FFU, about 1×10⁸ RCV FFU, or about 5×10⁸ RCV FFU.

Patients having HPV⁺ cancers usually encounter tumor progression or recurrence on standard-of care-therapies (e.g., failed radiation, platinum-based therapy, and/or anti-PD-1/anti-PD-L1 therapy), including the ones who went through more than one systemic therapy. On the other hand, some patients are contraindicated for standard-of-care therapies. Therefore, in some embodiments, the method provided herein include treating a patient who had tumor progression or recurrence on at least one standard-of-care therapy prior to the method. In some specific embodiments, the method provided herein are used to treat a patient who had failed radiation. In some specific embodiments, the method provided herein are used to treat a patient who had failed platinum-based therapy. In some specific embodiments, the method provided herein are used to treat a patient who had failed anti-PD-1 therapy. In some specific embodiments, the method provided herein are used to treat a patient who had failed anti-PD-L1 therapy. In one specific embodiment, the method provided herein are used to treat a patient who had failed pembrolizumab monotherapy. In one specific embodiment, the method provided herein are used to treat a patient who had failed nivolumab monotherapy. In one specific embodiment, the method provided herein are used to treat a patient who had failed pidilizumab monotherapy. In one specific embodiment, the method provided herein are used to treat a patient who had failed cemiplimab monotherapy. In some embodiments, the method provided herein are used to treat a patient who had failed a combination of two or more of the above-listed therapies.

In some embodiments, the methods provided herein include treating a patient whose lesions involve lymph nodes or not. Accordingly, in some embodiments, the methods provided herein include treating a patient who has only target lesions in lymph nodes. In some embodiments, the methods provided herein include treating a patient who has only target lesions in non-lymph nodes. In some embodiments, the methods provided herein include treating a patient who has target lesions in both lymph nodes and non-lymph nodes.

In some embodiments, the methods provided herein include administration of engineered replication-competent tri-segmented arenavirus particles using intravenous injection, intratumoral injection or a combination thereof. Accordingly, in some embodiments, the methods provided herein are for treating HPV 16⁺ cancer (e.g., head and neck squamous cell carcinoma, cervical cancer, anal cancer, vulvar or vaginal cancer) in a patient in need thereof by intravenous injection to the patient of an effective amount of engineered replication-competent tri-segmented arenavirus particles having duplicated (i.e., two) S-segments encoding a fusion protein of HPV16 E7/E6, wherein the effective amount is about 5×10⁵ RCV FFU, about 5×10⁶ RCV FFU, about 5×10⁷, about 1×10⁸ RCV FFU, or about 5×10⁸ RCV FFU. In some embodiments, the methods provided herein are for treating HPV 16⁺ cancer (e.g., head and neck squamous cell carcinoma, cervical cancer, anal cancer, vulvar, or vaginal cancer) in a patient in need thereof by intratumoral injection to the patient of an effective amount of engineered replication-competent tri-segmented arenavirus particles having duplicated (i.e. two) S-segments encoding a fusion protein of HPV16 E7/E6, wherein the effective amount is about 5×10⁵ RCV FFU, about 5×10⁶ RCV FFU, about 5×10⁷, about 1×10⁸ RCV FFU, about 5×10⁸ RCV FFU. In some embodiments, the methods provided herein are for treating HPV 16⁺ cancer (e.g., head and neck squamous cell carcinoma, cervical cancer, anal cancer, vulvar, or vaginal cancer) in a patient in need thereof by intratumoral injection to the patient of an effective amount of engineered replication-competent tri-segmented arenavirus particles having duplicated (i.e. two) S-segments encoding a fusion protein of HPV16 E7/E6, wherein the effective amount of the intratumoral injection is about 5×10⁵ RCV FFU, about 5×10⁶ RCV FFU, or about 5×10⁷, about 1×10⁸ RCV FFU, or about 5×10⁸ RCV FFU, followed by intravenous injection to the patient of an effective amount of engineered replication-competent tri-segmented arenavirus particles having duplicated (i.e., two) S-segments encoding a fusion protein of HPV16 E7/E6, wherein the effective amount intravenous injection is about 5×10⁵ RCV FFU, about 5×10⁶ RCV FFU, or about 5×10⁷, about 1×10⁸ RCV FFU, or about 5×10⁸ RCV FFU.

In some embodiments, the methods provided herein include administering intravenous injection to the patient of an effective amount of engineered replication-competent tri-segmented arenavirus particles having duplicated (i.e. two) S-segments encoding a fusion protein of HPV16 E7/E6, such as Construct 1, with a certain frequency (e.g., every 2 weeks, every 3 weeks, every 4 weeks, every 5 weeks, every 6 weeks, every 7 weeks, or every 8 weeks, every 9 weeks, every 10 weeks, every 11 weeks, every 12 weeks, or every 13 weeks, every 14 weeks, etc.). Accordingly, in some embodiments, the methods provided herein include administering intravenous injections to the patient of an effective amount of Construct 1 with a frequency of every 2 weeks. In some embodiments, the methods provided herein include administering intravenous injections to the patient of an effective amount of Construct 1 with a frequency of every 3 weeks. In some embodiments, the methods provided herein include administering intravenous injections to the patient of an effective amount of Construct 1 with a frequency of every 4 weeks. In some embodiments, the methods provided herein include administering intravenous injections to the patient of an effective amount of Construct 1 with a frequency of every 5 weeks. In some embodiments, the methods provided herein include administering intravenous injections to the patient of an effective amount of Construct 1 with a frequency of every 6 weeks. In some embodiments, the methods provided herein include administering intravenous injections to the patient of an effective amount of Construct 1 with a frequency of every 7 weeks. In some embodiments, the methods provided herein include administering intravenous injections to the patient of an effective amount of Construct 1 with a frequency of every 8 weeks. In some embodiments, the methods provided herein include administering intravenous injections to the patient of an effective amount of Construct 1 with a frequency of every 9 weeks. In some embodiments, the methods provided herein include administering intravenous injections to the patient of an effective amount of Construct 1 with a frequency of every 10 weeks. In some embodiments, the methods provided herein include administering intravenous injections to the patient of an effective amount of Construct 1 with a frequency of every 11 weeks. In some embodiments, the methods provided herein include administering intravenous injections to the patient of an effective amount of Construct 1 with a frequency of every 12 weeks. In some embodiments, the methods provided herein include administering intravenous injections to the patient of an effective amount of Construct 1 with a frequency of every 13 weeks, every 14 weeks, every 15 weeks, every 16 weeks, every 17 weeks, every 18 weeks, every 19 weeks, every 20 weeks, every 21 weeks, every 22 weeks, every 23 weeks, every 24 weeks, every 25 weeks, or every 26 weeks. In a preferred embodiment, the methods provided herein include administering intravenous injections to the patient of 5×10⁶ RCV FFU of Construct 1 with a frequency of every 3 weeks.

In some embodiments, the methods provided herein include administering intravenous injection to the patient of an effective amount of engineered replication-competent tri-segmented arenavirus particles having duplicated (i.e. two) S-segments encoding a fusion protein of HPV16 E7/E6, such as Construct 2, with a certain frequency (e.g., every 2 weeks, every 3 weeks, every 4 weeks, every 5 weeks, every 6 weeks, every 7 weeks, or every 8 weeks, every 9 weeks, every 10 weeks, every 11 weeks, every 12 weeks, or every 13 weeks, every 14 weeks, etc.). Accordingly, in some embodiments, the methods provided herein include administering intravenous injections to the patient of an effective amount of Construct 2 with a frequency of every 2 weeks. In some embodiments, the methods provided herein include administering intravenous injections to the patient of an effective amount of Construct 2 with a frequency of every 3 weeks. In some embodiments, the methods provided herein include administering intravenous injections to the patient of an effective amount of Construct 2 with a frequency of every 4 weeks. In some embodiments, the methods provided herein include administering intravenous injections to the patient of an effective amount of Construct 2 with a frequency of every 5 weeks. In some embodiments, the methods provided herein include administering intravenous injections to the patient of an effective amount of Construct 2 with a frequency of every 6 weeks. In some embodiments, the methods provided herein include administering intravenous injections to the patient of an effective amount of Construct 2 with a frequency of every 7 weeks. In some embodiments, the methods provided herein include administering intravenous injections to the patient of an effective amount of Construct 2 with a frequency of every 8 weeks. In some embodiments, the methods provided herein include administering intravenous injections to the patient of an effective amount of Construct 2 with a frequency of every 9 weeks. In some embodiments, the methods provided herein include administering intravenous injections to the patient of an effective amount of Construct 2 with a frequency of every 10 weeks. In some embodiments, the methods provided herein include administering intravenous injections to the patient of an effective amount of Construct 2 with a frequency of every 11 weeks. In some embodiments, the methods provided herein include administering intravenous injections to the patient of an effective amount of Construct 2 with a frequency of every 12 weeks. In some embodiments, the methods provided herein include administering intravenous injections to the patient of an effective amount of Construct 2 with a frequency of every 13 weeks, every 14 weeks, every 15 weeks, every 16 weeks, every 17 weeks, every 18 weeks, every 19 weeks, every 20 weeks, every 21 weeks, every 22 weeks, every 23 weeks, every 24 weeks, every 25 weeks, or every 26 weeks.

In some embodiments, the methods provided herein include an ongoing treatment. In other embodiments, the methods provided herein include a treatment administered for a limited number of times. Accordingly, in some embodiments, the methods provided herein include administering intravenous injections to the patient of an effective amount of engineered replication-competent tri-segmented arenavirus particles having duplicated (i.e. two) S-segments encoding a fusion protein of HPV16 E7/E6, such as Construct 1 or Construct 2, with a certain frequency and ongoing. In some embodiments, the methods provided herein include administering intravenous injections to the patient of an effective amount of engineered replication-competent tri-segmented arenavirus particles having duplicated (i.e. two) S-segments encoding a fusion protein of HPV16 E7/E6, such as Construct 1 or Construct 2, with a certain frequency for a limited number of times.

Specifically, for the methods only administered for a limited number of cycles, several factors, such as a proper number of cycles, a proper dosage, and a dosing frequency, need to be optimized to achieve the same therapeutic effect as the methods administered in an ongoing manner. Accordingly, in some embodiments, the methods provided herein include administering intravenous injections to the patient of an effective amount of engineered replication-competent tri-segmented arenavirus particles having duplicated (i.e. two) S-segments encoding a fusion protein of HPV16 E7/E6, such as Construct 1 or Construct 2, every 2 weeks for only 2 cycles, every 3 weeks for only 2 cycles, every 4 weeks for only 2 cycles, every 5 weeks for only 2 cycles, every 6 weeks for only 2 cycles, every 7 weeks for only 2 cycles, every 8 weeks for only 2 cycles, every 9 weeks for only 2 cycles, every 10 weeks for only 2 cycles, every 11 weeks for only 2 cycles, every 12 weeks for only 2 cycles. In some embodiments, the methods provided herein include administering intravenous injections to the patient of an effective amount of engineered replication-competent tri-segmented arenavirus particles having duplicated (i.e. two) S-segments encoding a fusion protein of HPV16 E7/E6, such as Construct 1 or Construct 2, every 2 weeks for only 3 cycles, every 3 weeks for only 3 cycles, every 4 weeks for only 3 cycles, every 5 weeks for only 3 cycles, every 6 weeks for only 3 cycles, every 7 weeks for only 3 cycles, every 8 weeks for only 3 cycles, every 9 weeks for only 3 cycles, every 10 weeks for only 3 cycles, every 11 weeks for only 3 cycles, every 12 weeks for only 3 cycles. In some embodiments, the methods provided herein include administering intravenous injections to the patient of an effective amount of engineered replication-competent tri-segmented arenavirus particles having duplicated (i.e. two) S-segments encoding a fusion protein of HPV16 E7/E6, such as Construct 1 or Construct 2, every 2 weeks for only 4 cycles, every 3 weeks for only 4 cycles, every 4 weeks for only 4 cycles, every 5 weeks for only 4 cycles, every 6 weeks for only 4 cycles, every 7 weeks for only 4 cycles, every 8 weeks for only 4 cycles, every 9 weeks for only 4 cycles, every 10 weeks for only 4 cycles, every 11 weeks for only 4 cycles, every 12 weeks for only 4 cycles. In some embodiments, the methods provided herein include administering intravenous injections to the patient of an effective amount of engineered replication-competent tri-segmented arenavirus particles having duplicated (i.e. two) S-segments encoding a fusion protein of HPV16 E7/E6, such as Construct 1 or Construct 2, every 2 weeks for only 5 cycles, every 3 weeks for only 5 cycles, every 4 weeks for only 5 cycles, every 5 weeks for only 5 cycles, every 6 weeks for only 5 cycles, every 7 weeks for only 5 cycles, every 8 weeks for only 5 cycles, every 9 weeks for only 5 cycles, every 10 weeks for only 5 cycles, every 11 weeks for only 5 cycles, every 12 weeks for only 5 cycles. Furthermore, the methods provided herein include administering intravenous injections to the patient of an effective amount of Construct 1 with a limited number of cycles, wherein the effective amount can be 5×10⁶, 5×10⁷, 5×10⁸, 1×10⁹, or 5×10⁹ RCV FFU. Accordingly, in some embodiments, the methods provided herein include administering intravenous injections to the patient of 5×10⁶ RCV FFU of Construct 1 with a limited number of cycles as described above in the same paragraph. In some embodiments, the methods provided herein include administering intravenous injections to the patient of 5×10⁷ RCV FFU of Construct 1 with a limited number of cycles as described above in the same paragraph. In some embodiments, the methods provided herein include administering intravenous injections to the patient of 5×10⁸ RCV FFU of Construct 1 with a limited number of cycles as described above in the same paragraph. In some embodiments, the methods provided herein include administering intravenous injections to the patient of 1×10⁹ RCV FFU of Construct 1 with a limited number of cycles as described above in the same paragraph. In some embodiments, the methods provided herein include administering intravenous injections to the patient of 5×10⁹ RCV FFU of Construct 1 with a limited number of cycles as described above in the same paragraph. Furthermore, the methods provided herein include administering intravenous injections to the patient of an effective amount of Construct 2 with a limited number of cycles, wherein the effective amount can be 1×10⁷, 1×10⁸, 1×10⁹, or 1×10¹⁰ RCV FFU. Accordingly, in some embodiments, the methods provided herein include administering intravenous injections to the patient of 1×10⁷ RCV FFU of Construct 2 with a limited number of cycles as described above in the same paragraph. In some embodiments, the methods provided herein include administering intravenous injections to the patient of 1×10⁸ RCV FFU of Construct 2 with a limited number of cycles as described above in the same paragraph. In some embodiments, the methods provided herein include administering intravenous injections to the patient of 1×10⁹ RCV FFU of Construct 2 with a limited number of cycles as described above in the same paragraph. In some embodiments, the methods provided herein include administering intravenous injections to the patient of 1×10¹⁰ RCV FFU of Construct 2 with a limited number of cycles as described above in the same paragraph.

In some embodiments, the methods provided herein can also include administering an effective amount of an immune checkpoint inhibitor. An exemplary immune checkpoint inhibitor that is particularly useful for use in the methods described herein include an anti-PD-1 (programmed cell death protein 1) checkpoint inhibitor. Such an anti-PD-1 checkpoint inhibitor can be an antibody, such as nivolumab, pembrolizumab, pidilizumab or cemiplimab. Accordingly, in some embodiments, the methods provided herein are for treating HPV 16⁺ cancer (e.g., head and neck squamous cell carcinoma, cervical cancer, anal cancer, vulvar, or vaginal cancer) in a patient in need thereof by administering to the patient an effective amount of an immune checkpoint inhibitor and an effective amount of an engineered replication-competent tri-segmented arenavirus particles having duplicated (i.e., two) S-segments encoding a fusion protein of HPV16 E7/E6, wherein the effective amount of the particle is about 5×10⁵ RCV FFU, about 5×10⁶ RCV FFU, about 5×10⁷ RCV FFU, about 1×10⁸ RCV FFU, or about 5×10⁸ RCV FFU, and wherein the immune checkpoint inhibitor is an anti-PD-1 checkpoint inhibitor (e.g., nivolumab, pembrolizumab, pidilizumab or cemiplimab). In some embodiments, the anti-PD-1 checkpoint inhibitor is nivolumab. In some embodiments, the anti-PD-1 checkpoint inhibitor is pembrolizumab. In some embodiments, the anti-PD-1 checkpoint inhibitor is pidilizumab. In some embodiments, the anti-PD-1 checkpoint inhibitor is cemiplimab.

Specifically, for the methods administered in an ongoing manner, several factors, such as a proper dosing frequency that starts with a higher frequency and followed by a lower frequency, and a proper number of cycles with the initial higher frequency (each administration is defined as a “cycle”), need to be optimized. Accordingly, in some embodiments, the methods provided herein include administering intravenous injection to the patient of an effective amount of engineered replication-competent tri-segmented arenavirus particles having duplicated (i.e. two) S-segments encoding a fusion protein of HPV16 E7/E6, such as Construct 1 or Construct 2, with a higher frequency followed by a lower frequency. Therefore, in some embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 2 weeks for 2, 3, 4, 5, 6, 7, 8, 9, 10, 11, or 12 cycles followed by a frequency of every 3 weeks, every 4 weeks, every 5 weeks, every 6 weeks, every 7 weeks, every 8 weeks, every 9 weeks, every 10 weeks, every 11 weeks, or every 12 weeks. In some embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 3 weeks for 2, 3, 4, 5, 6, 7, 8, 9, 10, 11, or 12 cycles followed by a frequency of every 4 weeks, every 5 weeks, every 6 weeks, every 7 weeks, every 8 weeks, every 9 weeks, every 10 weeks, every 11 weeks, every 12 weeks, or every 13 week. In some embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 4 weeks for 2, 3, 4, 5, 6, 7, 8, 9, 10, 11, or 12 cycles followed by a frequency of every 5 weeks, every 6 weeks, every 7 weeks, every 8 weeks, every 9 weeks, every 10 weeks, every 11 weeks, every 12 weeks, every 13 weeks, or every 14 weeks. In some embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 5 weeks for 2, 3, 4, 5, 6, 7, 8, 9, 10, 11, or 12 cycles followed by a frequency of every 6 weeks, every 7 weeks, every 8 weeks, every 9 weeks, every 10 weeks, every 11 weeks, every 12 weeks, every 13 weeks, every 14 weeks, or every 15 weeks. In some embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 6 weeks for 2, 3, 4, 5, 6, 7, 8, 9, 10, 11, or 12 cycles followed by a frequency of every 7 weeks, every 8 weeks, every 9 weeks, every 10 weeks, every 11 weeks, every 12 weeks, every 13 weeks, every 14 weeks, every 15 weeks, or every 16 weeks. In some preferred embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 3 weeks for 4 cycles followed by a frequency of every 6 weeks. In other preferred embodiments, the methods provided herein include administering intravenous injections to the patient of an effective amount of Construct 1 (e.g., 5×10⁵, 5×10⁶, 5×10⁷, 1×10⁸, or 5×10⁸ RCV FFU) with a frequency of every 4 weeks for 4 cycles followed by a frequency of every 8 weeks. In some embodiments, the methods provided herein include administering intravenous injections to the patient of an effective amount of Construct 2 (e.g., 1×10⁶, 1×10⁷, 1×10⁸, or 1×10⁹ RCV FFU) with a frequency of every 2 weeks for 2, 3, 4, 5, 6, 7, 8, 9, 10, 11, or 12 cycles followed by a frequency of every 3 weeks, every 4 weeks, every 5 weeks, every 6 weeks, every 7 weeks, every 8 weeks, every 9 weeks, every 10 weeks, every 11 weeks, or every 12 weeks. In some embodiments, the methods provided herein include administering intravenous injections to the patient of an effective amount of Construct 2 (e.g., 1×10⁶, 1×10⁷, 1×10⁸, or 1×10⁹ RCV FFU) with a frequency of every 3 weeks for 2, 3, 4, 5, 6, 7, 8, 9, 10, 11, or 12 cycles followed by a frequency of every 4 weeks, every 5 weeks, every 6 weeks, every 7 weeks, every 8 weeks, every 9 weeks, every 10 weeks, every 11 weeks, every 12 weeks, or every 13 week. In some embodiments, the methods provided herein include administering intravenous injections to the patient of an effective amount of Construct 2 (e.g., 1×10⁶, 1×10⁷, 1×10⁸, or 1×10⁹ RCV FFU) with a frequency of every 4 weeks for 2, 3, 4, 5, 6, 7, 8, 9, 10, 11, or 12 cycles followed by a frequency of every 5 weeks, every 6 weeks, every 7 weeks, every 8 weeks, every 9 weeks, every 10 weeks, every 11 weeks, every 12 weeks, every 13 weeks, or every 14 weeks. In some embodiments, the methods provided herein include administering intravenous injections to the patient of an effective amount of Construct 2 (e.g., 1×10⁶, 1×10⁷, 1×10⁸, or 1×10⁹ RCV FFU) with a frequency of every 5 weeks for 2, 3, 4, 5, 6, 7, 8, 9, 10, 11, or 12 cycles followed by a frequency of every 6 weeks, every 7 weeks, every 8 weeks, every 9 weeks, every 10 weeks, every 11 weeks, every 12 weeks, every 13 weeks, every 14 weeks, or every 15 weeks. In some embodiments, the methods provided herein include administering intravenous injections to the patient of an effective amount of Construct 2 (e.g., 1×10⁶, 1×10⁷, 1×10⁸, or 1×10⁹ RCV FFU) with a frequency of every 6 weeks for 2, 3, 4, 5, 6, 7, 8, 9, 10, 11, or 12 cycles followed by a frequency of every 7 weeks, every 8 weeks, every 9 weeks, every 10 weeks, every 11 weeks, every 12 weeks, every 13 weeks, every 14 weeks, every 15 weeks, or every 16 weeks.

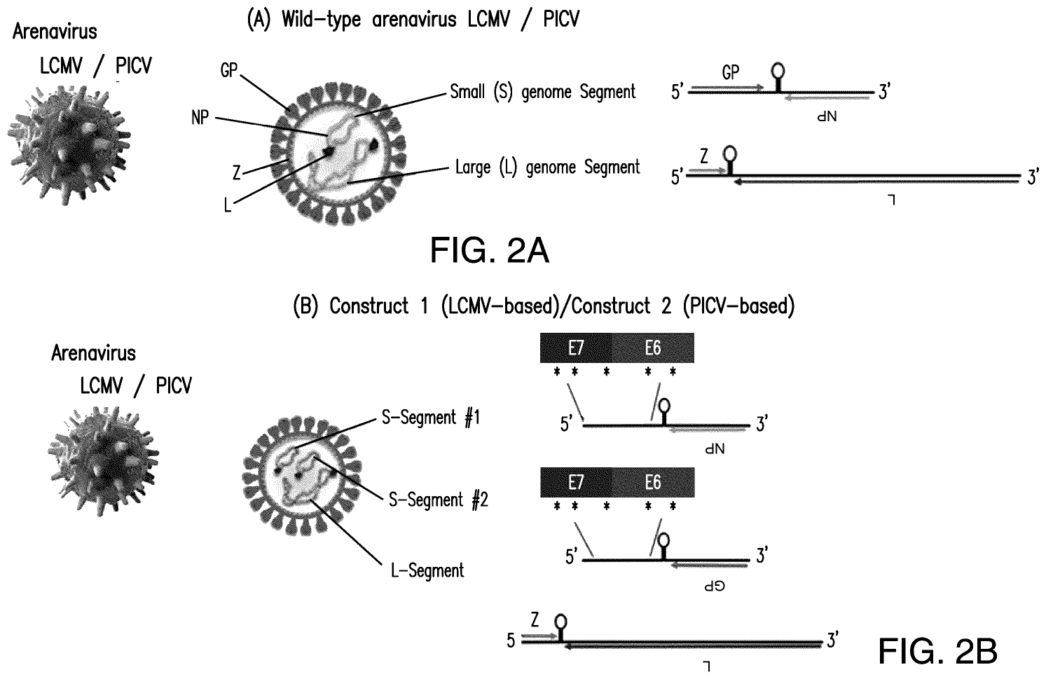

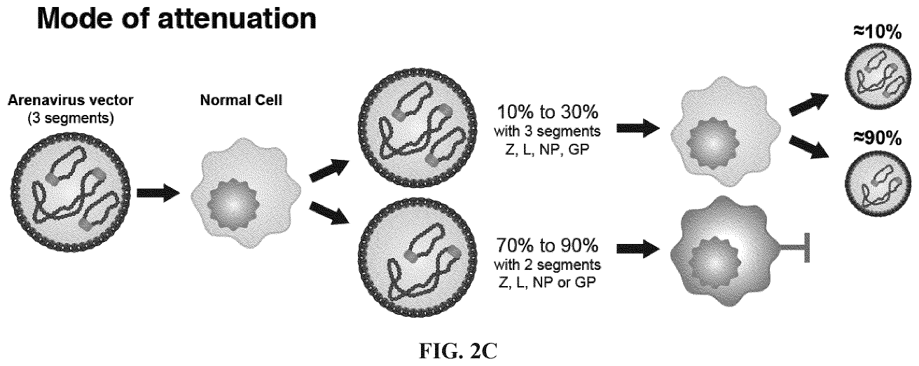

In some embodiments, the engineered replication-competent tri-segmented arenavirus particles used in the methods described herein include the arenavirus particle of Construct 1 (LCMV-based) as described herein (FIG. 2B and Examples I and II). In addition, in some embodiments, the engineered replication-competent tri-segmented arenavirus particles used in the methods described herein are derived from LCMV. Specific strains of LCMV include MP strain, WE strain, Armstrong strain, Armstrong Clone 13 strain, or LCMV clone 13 strain expressing the glycoprotein of LCMV strain WE instead of endogenous LCMV clone 13 glycoprotein. Accordingly, in some embodiments, the engineered replication-competent tri-segmented arenavirus particles used in the methods described herein are derived from LCMV MP strain. In some embodiments, the engineered replication-competent tri-segmented arenavirus particles used in the methods described herein are derived from LCMV WE strain. In some embodiments, the engineered replication-competent tri-segmented arenavirus particles used in the methods described herein are derived from LCMV Armstrong strain. In some embodiments, the engineered replication-competent tri-segmented arenavirus particles used in the methods described herein are derived from LCMV Armstrong Clone 13 strain. In some embodiments, the engineered replication-competent tri-segmented arenavirus particles used in the methods described herein are derived from LCMV clone 13 strain expressing the glycoprotein of LCMV strain WE instead of endogenous LCMV clone 13 glycoprotein.

In other embodiments, the engineered replication-competent tri-segmented arenavirus particles used in the methods described herein are derived from Construct 2 (PICV-based) as described herein (FIG. 2B and Example II). Specific strains of PICV include strain Munchique CoAn4763 isolate P18, P2 strain, or any of the several isolates described by Trapido and colleagues (Trapido et al., 1971, Am J Trop Med Hyg, 20: 631-641). Accordingly, in some embodiments, the engineered replication-competent tri-segmented arenavirus particles used in the methods described herein are derived from strain Munchique CoAn4763 isolate P18. In some embodiments, the engineered replication-competent tri-segmented arenavirus particles used in the methods described herein are derived from P2 strain. In some embodiments, the engineered replication-competent tri-segmented arenavirus particles used in the methods described herein are derived from any of the several isolates described by Trapido and colleagues (Trapido et al., 1971, Am J Trop Med Hyg, 20: 631-641)

Certain cytokines and chemokines are measured after the administration of the effective amount of engineered replication-competent tri-segmented arenavirus particles. The cytokines and chemokines to be measured include pro-inflammatory and anti-inflammatory cytokines and chemokines.

Therefore, further provided herein is a method for treating cancer in a patient in need thereof comprising administering to the patient an effective amount of engineered replication-competent tri-segmented arenavirus particles comprising duplicated (i.e., two) S-segments encoding a fusion protein of HPV16 E7/E6, wherein the effective amount is about 5×10⁵, about 5×10⁶ RCV FFU, about 5×10⁷ RCV FFU, about 1×10⁸ RCV FFU, or about 5×10⁸ RCV FFU, wherein the administration of the effective amount of engineered replication-competent tri-segmented arenavirus particles results in a change (i.e., increase or decrease) in the level of a cytokine or a chemokine in the serum of the patient as compared to the pre-treatment level in the patient.

In some specific embodiments, the changed cytokines and chemokines (i.e., increased or decreased cytokines and chemokines) described herein include, but are not limited to, GM-CSF, IL-1α, IL-1, IL-2, IL-4, IL-5, IL-6, IL-7, IL-8, IL-10, IL-11, IL-12, IL-12p40, IL-15, IL-16, IL-17, IL-17A, IL-18, IL-22, IL-37, IL-38, TGF-β, IFN-α, INF-β, IFN-γ, TNF-α, TNF-β, IFN-inducible protein (IP)-10, macrophage inflammatory protein (MIP)-1α, MIP-1β, monocyte chemoattractant protein (MCP)-1, MCP-4, eotaxin, eotaxin-3, thymus and activation-regulated chemokine (TARC), macrophage-derived chemokine (MDC), and vascular endothelial growth factor (VEGF).

In some embodiments, the cytokines and chemokines described herein have pro-inflammatory and/or anti-inflammatory activities. In some embodiments, the administration of the effective amount of engineered replication-competent tri-segmented arenavirus particles results in a 2-fold, 3-fold, 4-fold, 5-fold, 6-fold, 7-fold, 8-fold, 9-fold, 10-fold, 11-fold, 12-fold, 13-fold, 14-fold, 15-fold, 20-fold, 21-fold, 22-fold, 23-fold, 24-fold, 25-fold, 26-fold, 27-fold, 28-fold, 29-fold, or 30-fold change (e.g., increase or decrease) in the level of a cytokine or a chemokine (e.g., having pro-inflammatory and/or anti-inflammatory activity) in the serum of the patient as compared to the pre-treatment level in the patient. In other embodiments, the administration of the effective amount of engineered replication-competent tri-segmented arenavirus particles results in a 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold change (e.g., increase or decrease) in the level of a cytokine or a chemokine (e.g., having pro-inflammatory and/or anti-inflammatory activity) in the serum of the patient as compared to the pre-treatment level in the patient. In other embodiments, the administration of the effective amount of engineered replication-competent tri-segmented arenavirus particles results in a 300-fold, 400-fold, 500-fold, 600-fold, 700-fold, 800-fold, 900-fold, 1000-fold change (e.g., increase or decrease) in the level of a cytokine or a chemokine (e.g., having pro-inflammatory and/or anti-inflammatory activity) in the serum of the patient as compared to the pre-treatment level in the patient.

In some embodiments, the cytokines and chemokines described herein serve as biomarkers for patient population selection. Accordingly, in some embodiments, a measurement of one or more of the cytokines and chemokines described herein above a certain threshold in a patient prior to the treatment indicates the patient is suitable for the methods provided herein. In some embodiments, a measurement of one or more of the cytokines and chemokines described herein below a certain threshold in a patient prior to the treatment indicates the patient is suitable for the methods provided herein. In specific embodiments, a measurement of IFN-γ, IL-12p40, IL-15, IFN-inducible protein (IP)-10, TNFα, or a combination thereof below a certain threshold in a patient prior to the treatment indicates the patient is suitable for the methods provided herein.

In some embodiments, the cytokines and chemokines described herein serve as biomarkers for re-adjusting the doses and/or regimens during the treatment. Accordingly, in some embodiments, after treating a patient for a period of time, a measurement of one or more of the cytokines and chemokines described herein above a certain threshold indicates an increase of doses and/or frequency of administration. In some embodiments, after treating a patient for a period of time, a measurement of one or more of the cytokines and chemokines described herein above a certain threshold indicates keeping the same doses and/or frequency of administration. In some embodiments, after treating a patient for a period of time, a measurement of one or more of the cytokines and chemokines described herein above a certain threshold indicates a decrease of doses and/or frequency of administration. In some embodiments, after treating a patient for a period of time, a measurement of one or more of the cytokines and chemokines described herein below a certain threshold indicates an increase of doses and/or frequency of administration. In some embodiments, after treating a patient for a period of time, a measurement of one or more of the cytokines and chemokines described herein below a certain threshold indicates keeping the same doses and/or frequency of administration. In some embodiments, after treating a patient for a period of time, a measurement of one or more of the cytokines and chemokines described herein below a certain threshold indicates a decrease of doses and/or frequency of administration. In some specific embodiments, after treating a patient for a period of time, a measurement of IFN-γ, IL-12p40, IL-15, IFN-inducible protein (IP)-10, TNFα, or a combination thereof below a certain threshold indicates an increase of doses and/or frequency of administration. In some specific embodiments, after treating a patient for a period of time, a measurement of IFN-γ, IL-12p40, IL-15, IFN-inducible protein (IP)-10, TNFα, or a combination thereof above a certain threshold indicates keeping the same doses and/or frequency of administration.

The levels of the cytokines and chemokines can be measured at different time points before and after administering the engineered replication-competent tri-segmented arenavirus particles comprising duplicated (i.e., two) S-segments encoding a fusion protein of HPV16 E7/E6. In some embodiments, the levels of the cytokines and chemokines are measured before the administration of the arenavirus particles. In some embodiments, the levels of the cytokines and chemokines are measured 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours after the administration of the arenavirus particles. In some embodiments, the levels of the cytokines and chemokines are measured 2 days, 3 days, 4 days, 5 days, 6 days, 7 days, 8 days, 9 days, 10 days, 11 days, 12 days, 13 days, 15 days, 16 days, 17 days, 18 days, 19 days, 20 days, 21 days, 22 days, 23 days, 24 days, 25 days, 30 days, 35 days, 40 days, 45 days, 50 days, 55 days, 60 days, 65 days, 70 days, 75 days, 80 days, 85 days, 90 days, 95 days, 100 days, 150 days, 300 days, 450 days after the administration of the arenavirus particles.

The levels of the cytokines and chemokines can be measured with varieties of assays, such as bioassays (e.g., tests for chemotactic activity, proliferation, or cytotoxicity), immunoassays (e.g., ELISA, and especially multiplex ELISA), flow cytometry, and aptamers-based detection methods, and molecular imaging with radiolabeled cytokines and chemokines. Accordingly, in some embodiments, after the administration of the arenavirus particles encoding HPV16 E7/E6 the cytokines and chemokines are measured with bioassays (e.g., tests for chemotactic activity, proliferation, or cytotoxicity). In some embodiments, after the administration of the arenavirus particles encoding HPV16 E7/E6 the cytokines and chemokines are measured with immunoassays. In specific embodiments, after the administration of the arenavirus particles encoding HPV16 E7/E6 the cytokines and chemokines are measured with ELISA. In some preferred embodiments, after the administration of the arenavirus particles encoding HPV16 E7/E6 the cytokines and chemokines are measured with multiplex ELISA. In some embodiments, after the administration of the arenavirus particles encoding HPV16 E7/E6 the cytokines and chemokines are measured with flow cytometry. In some embodiments, after the administration of the arenavirus particles encoding HPV16 E7/E6 the cytokines and chemokines are measured with aptamers-based detection methods. In some embodiments, after the administration of the arenavirus particles encoding HPV16 E7/E6 the cytokines and chemokines are measured with molecular imaging with radiolabeled cytokines and chemokines.

Further provided herein is a method for treating cancer in a patient in need thereof comprising administering to the patient an effective amount of engineered replication-competent tri-segmented arenavirus particles comprising duplicated (i.e., two) S-segments encoding a fusion protein of human papillomavirus strain 16 (HPV16) E7/E6, wherein the effective amount is about 5×10⁵, about 5×10⁶ RCV FFU, about 5×10⁷ RCV FFU, about 1×10⁸ RCV FFU, or about 5×10⁸ RCV FFU, wherein the administration of the effective amount of engineered replication-competent tri-segmented arenavirus particles results in a change in cytokine or chemokine levels in the serum of the patient as compared to the pre-treatment level of the patient, and wherein the administration of the effective amount of engineered replication-competent tri-segmented arenavirus particles results in an increase of HPV16 E7/E6-specific T cells in the serum of the patient as compared to the pre-treatment level of the patient.

The increased HPV16 E7/E6-specific T cells described herein can be positive for different cellular markers (e.g., CD4, CD8, IFN-γ, TNFα, CD107a) alone or in combination which indicate the different functionalities of the T cells. In some embodiments, the method provided herein results in an increase of HPV16 E7/E6-specific T cells that are positive for CD4 in the serum of the patient as compared to the pre-treatment level of the patient. In some embodiments, the method provided herein results in an increase of HPV16 E7/E6-specific T cells that are positive for CD8 in the serum of the patient as compared to the pre-treatment level of the patient. In some embodiments, the method provided herein results in an increase of HPV16 E7/E6-specific T cells that are positive for IFN-γ in the serum of the patient as compared to the pre-treatment level of the patient. In some embodiments, the method provided herein results in an increase of HPV16 E7/E6-specific T cells that are positive for TNFα in the serum of the patient as compared to the pre-treatment level of the patient. In some embodiments, the method provided herein results in an increase of HPV16 E7/E6-specific T cells that are positive for CD107a in the serum of the patient as compared to the pre-treatment level of the patient.

The HPV16 E7/E6-specific T cells can be detected and quantified by varieties of assays, such as ELISpot and intracellular cytokine staining (ICS) followed by flow cytometry. Accordingly, in some embodiments, the method provided herein further comprises detecting and quantifying HPV16 E7/E6-specific T cells by ELISpot. In some embodiments, the method provided herein further comprises detecting and quantifying HPV16 E7/E6-specific T cells by ICS followed by flow cytometry.

Specifically, any assay well known in the art can be used to test HPV16 E7/E6-specific T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8). Cytokines such as but not limited to IFN-γ can be measured by the ELISPOT assay. Briefly, the assay comprises the following steps: An immunospot plate is coated with an anti-cytokine antibody. Cells are incubated in the immunospot plate with peptides derived from HPV E7/E6. HPV16 E7/E6-specific cells secrete cytokines, which bind to the coated antibodies. The cells are then washed off and a second biotyinlated-anticytokine antibody is added to the plate and visualized with an avidin-HRP system or other appropriate methods.

Similarly, any assay well known in the art can be used to test the functionality of CD8⁺ and CD4⁺ T cells that are specific for HPV16 E7/E6. For example, the ICS combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: upon activation of cells via HPV16 E7/E6, an inhibition of protein transport (e.g., brefeldin A) is added to retain the cytokines within the cell. After a defined period of incubation, typically 5 hours, a washing step follows, and antibodies to other cellular markers can be added to the cells. Cells are then fixed and permeabilized. The flurochrome-conjugated anti-cytokine antibodies are added and the cells can be analyzed by flow cytometry.

It is well known in the art that the frequency of target antigen-specific T cells induced in patients by cancer immunotherapies is usually too low to directly measure these responses without prior in-vitro expansion. Therefore, it is a common practice in the art to perform in-vitro T cell stimulation and/or expansion before detection (see e.g., Trickett et al., J Immunol Methods. 2003 Apr. 1; 275(1-2):251-5.). In direct contrast, for the T cell detection described in the preceding paragraphs, the increased T cell frequencies described herein are detected without prior in-vitro stimulation and/or expansion. Specifically, fresh or cryopreserved but thawed peripheral blood mononuclear cells (PBMCs) from treated patients are directly used for detection without prior in vitro expansion (see e.g., EXAMPLE II and EXAMPLE V).

Apart from the detection of abundance and functionality of induced T cells as described in the preceding paragraphs, the migration of T cells to infiltrate into tumor tissues is also an important readout for the efficacy of the methods provided herein. In some embodiments, the methods provided herein result in more T cells infiltrated into tumor tissues. In some specific embodiments, the methods provided herein result in more CD8⁺ T cells infiltrated into HPV16⁺ tumor tissues as compared to the pre-treatment level of the patient or patients receiving placebo (see section 11 of EXAMPLE III).

In addition, other assays for determining the humoral immune response upon vaccination can be done by antigen-specific serum ELISA's (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

Furthermore, other assays such as determining the neutralizing antibodies in sera can be performed with the following cell assay using GFP-tagged viruses or cell lines expressing HPV E7/E6. In addition supplemental guinea pig serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope. Similarly, neutralizing activity of induced antibodies can be measured in clinical setting.

In some embodiments, the methods provided herein result in one or more improved efficacy endpoint (e.g., percentage of objective response rate, percentage of disease control rate, percentage of partial response, progression-free survival, and/or overall survival) using Response Evaluation Criteria in Solid Tumors (RECIST) and/or Immune Response Evaluation Criteria in Solid Tumors (iRECIST), compared to the pre-treatment level of the patient or patients receiving placebo. Accordingly, in some specific embodiments, the methods provided herein result in higher percentage of objective response rate compared to the pre-treatment level of the patient or patients receiving placebo. In some specific embodiments, the methods provided herein result in higher percentage of disease control rate compared to the pre-treatment level of the patient or patients receiving placebo. In some specific embodiments, the methods provided herein result in higher percentage of partial response compared to the pre-treatment level of the patient or patients receiving placebo. In some specific embodiments, the methods provided herein result in longer progression-free survival compared to the pre-treatment level of the patient or patients receiving placebo. In some specific embodiments, the methods provided herein result in longer overall survival compared to the pre-treatment level of the patient or patients receiving placebo.

Further provided herein is a method for treating cancer in a patient in need thereof comprising (i) administering to the patient an effective amount of engineered replication-competent tri-segmented arenavirus particles comprising duplicated (i.e., two) S-segments encoding a fusion protein of HPV16 E7/E6 derived from LCMV, wherein the effective amount is about 5×10⁵, 5×10⁶, 5×10⁷, or 5×10⁸ RCV FFU; and (ii) administering to the patient an effective amount of engineered replication-competent tri-segmented arenavirus particles comprising duplicated (i.e. two) S-segments encoding a fusion protein of HPV16 E7/E6 derived from PICV, wherein the effective amount is about 5×10⁵, 5×10⁶, 5×10⁷, or 5×10⁸ RCV FFU. Also provided herein is a method for treating cancer in a patient in need thereof comprising (i) administering to the patient an effective amount of engineered replication-competent tri-segmented arenavirus particles comprising duplicated (i.e., two) S-segments encoding a fusion protein of HPV16 E7/E6 derived from PICV, wherein the effective amount is about 5×10⁵, 5×10⁶, 5×10⁷, or 5×10⁸ RCV FFU; and (ii) administering to the patient an effective amount of engineered replication-competent tri-segmented arenavirus particles comprising duplicated (i.e., two) S-segments encoding a fusion protein of HPV16 E7/E6 derived from LCMV, wherein the effective amount is about 5×10⁵, 5×10⁶, 5×10⁷, or 5×10⁸ RCV FFU.

Further provided herein is a method for treating cancer in a patient in need thereof comprising (i) administering to the patient an effective amount of engineered replication-competent tri-segmented arenavirus particles comprising duplicated (i.e., two) S-segments encoding a fusion protein of HPV16 E7/E6 derived from LCMV, wherein the effective amount is about 5×10⁵, 5×10⁶, 5×10⁷, or 5×10⁸ RCV FFU; (ii) administering to the patient an effective amount of engineered replication-competent tri-segmented arenavirus particles comprising duplicated (i.e., two) S-segments encoding a fusion protein of HPV16 E7/E6 derived from PICV, wherein the effective amount is about 5×10⁵, 5×10⁶, 5×10⁷, or 5×10⁸ RCV FFU, and repeating (i) and (ii) for 1 time, 2 times, 3 times, 4 times, 5 times, 6 times, 7 times, 8 times, 9 times, or 10 times. Also provided herein is a method for treating cancer in a patient in need thereof comprising (i) administering to the patient an effective amount of engineered replication-competent tri-segmented arenavirus particles comprising duplicated (i.e., two) S-segments encoding a fusion protein of HPV16 E7/E6 derived from PICV, wherein the effective amount is about 5×10⁵, 5×10⁶, 5×10⁷, or 5×10⁸ RCV FFU; (ii) administering to the patient an effective amount of engineered replication-competent tri-segmented arenavirus particles comprising duplicated (i.e., two) S-segments encoding a fusion protein of HPV16 E7/E6 derived from LCMV, wherein the effective amount is about 5×10⁵, 5×10⁶, 5×10⁷, or 5×10⁸ RCV FFU, and repeating (i) and (ii) for 1 time, 2 times, 3 times, 4 times, 5 times, 6 times, 7 times, 8 times, 9 times, or 10 times.

In some embodiments, the interval between the (i) and (ii) in the preceding paragraphs is 1 week, 2 weeks, 3 weeks, 4 weeks, 5 weeks, 6 weeks, 7 weeks, 8 weeks, 9 weeks, 10 weeks, 11 weeks, or 12 weeks. In other embodiments, the interval between the (i) and (ii) in the preceding paragraphs is 13 weeks, 14 weeks, 15 weeks, 16 weeks, 17 weeks, 18 weeks, 19 weeks, 20 weeks, 22 weeks, 23 weeks, or 24 weeks. In other embodiments, the interval between the (i) and (ii) in the preceding paragraphs is 25 weeks, 26 weeks, 27 weeks, 28 weeks, 29 weeks, 30 weeks, 31 weeks, 32 weeks, 33 weeks, 34 weeks, 35 weeks, or 36 weeks. Furthermore, during the repeats of (i) and (ii), the interval can be the same as the original cycle of (i) and (ii), or can be different from the original cycle of (i) and (ii). Accordingly, the interval between the (i) and (ii) in the repeats can be 1 week, 2 weeks, 3 weeks, 4 weeks, 5 weeks, 6 weeks, 7 weeks, 8 weeks, 9 weeks, 10 weeks.

Further provided herein is a method for treating cancer in a patient in need thereof comprising (i) administering to the patient an effective amount of engineered replication-competent tri-segmented arenavirus particles comprising duplicated (i.e., two) S-segments encoding a fusion protein of HPV16 E7/E6 derived from LCMV in combination with an immune checkpoint inhibitor, wherein the effective amount of the arenavirus particles is about 5×10⁵, 5×10⁶, 5×10⁷, or 5×10⁸ RCV FFU; (ii) administering to the patient an effective amount of engineered replication-competent tri-segmented arenavirus particles comprising duplicated (i.e., two) S-segments encoding a fusion protein of HPV16 E7/E6 derived from PICV in combination with an immune checkpoint inhibitor, wherein the effective amount is about 5×10⁵, 5×10⁶, 5×10⁷, or 5×10⁸ RCV FFU, without repeats or repeating (i) and (ii) for 1 time, 2 times, 3 times, 4 times, 5 times, 6 times, 7 times, 8 times, 9 times, or 10 times. Also provided herein is a method for treating cancer in a patient in need thereof comprising (i) administering to the patient an effective amount of engineered replication-competent tri-segmented arenavirus particles comprising duplicated (i.e., two) S-segments encoding a fusion protein of HPV16 E7/E6 derived from PICV in combination with an immune checkpoint inhibitor, wherein the effective amount is about 5×10⁵, 5×10⁶, 5×10⁷, or 5×10⁸ RCV FFU; and (ii) administering to the patient an effective amount of engineered replication-competent tri-segmented arenavirus particles comprising duplicated (i.e., two) S-segments encoding a fusion protein of HPV16 E7/E6 derived from LCMV in combination with an immune checkpoint inhibitor, wherein the effective amount is about 5×10⁵, 5×10⁶, 5×10⁷, or 5×10⁸ RCV FFU, without repeats or repeating (i) and (ii) for 1 time, 2 times, 3 times, 4 times, 5 times, 6 times, 7 times, 8 times, 9 times, or 10 times.

Further provided herein is a method for treating cancer in a patient in need thereof comprising one or more session, wherein each session comprises: (i) administering to the patient an effective amount of engineered replication-competent tri-segmented arenavirus particles comprising two S-segments encoding a fusion protein of human papillomavirus strain 16 (HPV16) E7/E6 derived from LCMV, wherein the effective amount is about 5×10⁵, 1×10⁶, 5×10⁶, 1×10⁷, 5×10⁷, 1×10⁸, 5×10⁸, or 1×10⁹ replication-competent virus focus-forming units (RCV FFU); and (ii) administering to the patient an effective amount of engineered replication-competent tri-segmented arenavirus particles comprising two S-segments encoding a fusion protein of HPV16 E7/E6 derived from PICV at a time point around half of the session, wherein the effective amount is about 5×10⁵, 1×10⁶, 5×10⁶, 1×10⁷, 5×10⁷, 1×10⁸, 5×10⁸, or 1×10⁹ RCV FFU. Further provided herein is also a method for treating cancer in a patient in need thereof comprising one or more session, wherein each session comprises: (i) administering to the patient an effective amount of engineered replication-competent tri-segmented arenavirus particles comprising two S-segments encoding a fusion protein of human papillomavirus strain 16 (HPV16) E7/E6 derived from PICV, wherein the effective amount is about 5×10⁵, 1×10⁶, 5×10⁶, 1×10⁷, 5×10⁷, 1×10⁸, 5×10⁸, or 1×10⁹ replication-competent virus focus-forming units (RCV FFU); and (ii) administering to the patient an effective amount of engineered replication-competent tri-segmented arenavirus particles comprising two S-segments encoding a fusion protein of HPV16 E7/E6 derived from LCMV at a time point around half of the session, wherein the effective amount is about 5×10⁵, 1×10⁶, 5×10⁶, 1×10⁷, 5×10⁷, 1×10⁸, 5×10⁸, or 1×10⁹ RCV FFU.

In some specific embodiments, the methods provided herein comprise one or more session, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU. In one specific embodiment, the methods provided herein comprise one or more session, wherein each session comprises: (i) administering intravenously to the patient 1×10⁶ RCV FFU of Construct 2; and (ii) administering intravenously to the patient 5×10⁶ RCV FFU of Construct 1, and each session lasts for 6 weeks. In other specific embodiments, the methods provided herein comprise one or more session, wherein each session comprises: (i) administering to the patient an effective amount of Construct 1, wherein the effective amount is about 5×10⁵, 5×10⁶, 5×10⁷, 1×10⁸, or 5×10⁸ RCV FFU; and (ii) administering to the patient an effective amount of Construct 2 at a time point around half of the session, wherein the effective amount is about 1×10⁶, 1×10⁷, 1×10⁸, or 1×10⁹ RCV FFU.

Session duration determines the interval between the administration of two consecutive doses of (i) in the preceding paragraphs. Accordingly, in some embodiments, each session provided herein lasts for 4 weeks. In some embodiments, each session provided herein lasts for 6 weeks. In some embodiments, each session provided herein lasts for 8 weeks. In some embodiments, each session provided herein lasts for 10 weeks. In some embodiments, each session provided herein lasts for 12 weeks. In some embodiments, each session provided herein lasts for 14 weeks. In some embodiments, each session provided herein lasts for 16 weeks. In some embodiments, each session provided herein lasts for 18 weeks. In some embodiments, each session provided herein lasts for 20 weeks. In some embodiments, each session provided herein lasts for 22 weeks. In some embodiments, each session provided herein lasts for 24 weeks. In some embodiments, each session provided herein lasts for 26 weeks. In some embodiments, each session provided herein lasts for 28 weeks. In some embodiments, each session provided herein lasts for 30 weeks. In some embodiments, each session provided herein lasts for 32, 34, 36, 38, 40, 42, 44, 46, 48, 50, or 52 weeks.

In some embodiments, the administration of (i) and (ii) in the preceding paragraphs comprises intravenous injection, intratumoral injection, or a combination of intravenous injection and intratumoral injection. Accordingly, in some embodiments, the methods provided herein comprise one or more session, wherein each session comprises: (i) administering intravenously, intratumorally, or a combination of intravenous injection and intratumoral injection to the patient an effective amount of Construct 2, wherein the effective amount is about 1×10⁶, 1×10⁷, 1×10⁸, or 1×10⁹ RCV FFU; and (ii) administering intravenously, intratumorally, or a combination of intravenous injection and intratumoral injection to the patient an effective amount of Construct 1 at a time point around half of the session, wherein the effective amount is about 5×10⁵, 5×10⁶, 5×10⁷, 1×10⁸, or 5×10⁸ RCV FFU. In some embodiments, the methods provided herein comprises one or more session, wherein each session comprises: (i) administering intravenously, intratumorally, or a combination of intravenous injection and intratumoral injection to the patient an effective amount of Construct 1, wherein the effective amount is about 5×10⁵, 5×10⁶, 5×10⁷, 1×10⁸, or 5×10⁸ RCV FFU; and (ii) administering intravenously, intratumorally, or a combination of intravenous injection and intratumoral injection to the patient an effective amount of Construct 2 at a time point around half of the session, wherein the effective amount is about 1×10⁶, 1×10⁷, 1×10⁸, or 1×10⁹ RCV FFU.

In some embodiments, the methods provided herein include an ongoing treatment. In other embodiments, the methods provided herein include a treatment administered for a limited number of times. Accordingly, in some embodiments, the methods provided herein comprise ongoing sessions or a limited number of sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU. In some embodiments, the methods provided herein comprise ongoing sessions or a limited number of sessions, wherein each session comprises: (i) administering to the patient an effective amount of Construct 1, wherein the effective amount is about 5×10⁵, 5×10⁶, 5×10⁷, 1×10⁸, or 5×10⁸ RCV FFU; and (ii) administering to the patient an effective amount of Construct 2 at a time point around half of the session, wherein the effective amount is about 1×10⁶, 1×10⁷, 1×10⁸, or 1×10⁹ RCV FFU.

Specifically, for the methods only administered for a limited number of times, several factors, such as a proper number of sessions (e.g., 2, 3, 4, or 5 sessions), a proper dosage, and a proper session duration, need to be optimized to achieve the same therapeutic effect as the methods administered in an ongoing manner. Accordingly, in some embodiments, the methods provided herein comprise only 2 sessions, wherein each session comprises: (i) administering to the patient an effective amount of Construct 2, wherein the effective amount is about 1×10⁷, 1×10⁸, 1×10⁹, or 1×10¹⁰ RCV FFU; and (ii) administering to the patient an effective amount of Construct 1 at a time point around half of the session, wherein the effective amount is about 5×10⁶, 5×10⁷, 5×10⁸, 1×10⁹, or 5×10⁹ RCV FFU, and wherein each session lasts for 4, 6, 8, 10, or 12 weeks. In some embodiments, the methods provided herein comprise only 3 sessions, wherein each session comprises: (i) administering to the patient an effective amount of Construct 2, wherein the effective amount is about 1×10⁷, 1×10⁸, 1×10⁹, or 1×10¹⁰ RCV FFU; and (ii) administering to the patient an effective amount of Construct 1 at a time point around half of the session, wherein the effective amount is about 5×10⁶, 5×10⁷, 5×10⁸, 1×10⁹, or 5×10⁹ RCV FFU, and wherein each session lasts for 4, 6, 8, 10, or 12 weeks. In some embodiments, the methods provided herein comprise only 4 sessions, wherein each session comprises: (i) administering to the patient an effective amount of Construct 2, wherein the effective amount is about 1×10⁷, 1×10⁸, 1×10⁹, or 1×10¹⁰ RCV FFU; and (ii) administering to the patient an effective amount of Construct 1 at a time point around half of the session, wherein the effective amount is about 5×10⁶, 5×10⁷, 5×10⁸, 1×10⁹, or 5×10⁹ RCV FFU, and wherein each session lasts for 4, 6, 8, 10, or 12 weeks. In some embodiments, the methods provided herein comprise only 5 sessions, wherein each session comprises: (i) administering to the patient an effective amount of Construct 2, wherein the effective amount is about 1×10⁷, 1×10⁸, 1×10⁹, or 1×10¹⁰ RCV FFU; and (ii) administering to the patient an effective amount of Construct 1 at a time point around half of the session, wherein the effective amount is about 5×10⁶, 5×10⁷, 5×10⁸, 1×10⁹, or 5×10⁹ RCV FFU, and wherein each session lasts for 4, 6, 8, 10, or 12 weeks. In other embodiments, the methods provided herein comprise only 2 sessions, wherein each session comprises: (i) administering to the patient an effective amount of Construct 1, wherein the effective amount is about 5×10⁶, 5×10⁷, 5×10⁸, 1×10⁹, or 5×10⁹ RCV FFU; and (ii) administering to the patient an effective amount of Construct 2 at a time point around half of the session, wherein the effective amount is about 1×10⁷, 1×10⁸, 1×10⁹, or 1×10¹⁰ RCV FFU, and wherein each session lasts for 4, 6, 8, 10, or 12 weeks. In other embodiments, the methods provided herein comprise only 3 sessions, wherein each session comprises: (i) administering to the patient an effective amount of Construct 1, wherein the effective amount is about 5×10⁶, 5×10⁷, 5×10⁸, 1×10⁹, or 5×10⁹ RCV FFU; and (ii) administering to the patient an effective amount of Construct 2 at a time point around half of the session, wherein the effective amount is about 1×10⁷, 1×10⁸, 1×10⁹, or 1×10¹⁰ RCV FFU, and wherein each session lasts for 4, 6, 8, 10, or 12 weeks. In other embodiments, the methods provided herein comprise only 4 sessions, wherein each session comprises: (i) administering to the patient an effective amount of Construct 1, wherein the effective amount is about 5×10⁶, 5×10⁷, 5×10⁸, 1×10⁹, or 5×10⁹ RCV FFU; and (ii) administering to the patient an effective amount of Construct 2 at a time point around half of the session, wherein the effective amount is about 1×10⁷, 1×10⁸, 1×10⁹, or 1×10¹⁰ RCV FFU, and wherein each session lasts for 4, 6, 8, 10, or 12 weeks. In other embodiments, the methods provided herein comprise only 5 sessions, wherein each session comprises: (i) administering to the patient an effective amount of Construct 1, wherein the effective amount is about 5×10⁶, 5×10⁷, 5×10⁸, 1×10⁹, or 5×10⁹ RCV FFU; and (ii) administering to the patient an effective amount of Construct 2 at a time point around half of the session, wherein the effective amount is about 1×10⁷, 1×10⁸, 1×10⁹, or 1×10¹⁰ RCV FFU, and wherein each session lasts for 4, 6, 8, 10, or 12 weeks.

Specifically, for the methods administered in an ongoing manner, several factors, such as a regimen that starts with proper shorter sessions and followed by proper longer sessions, and a proper amount of shorter sessions, need to be optimized.

Accordingly, after optimizing a regimen that starts with proper shorter sessions and followed by proper longer sessions, in some embodiments, the methods provided herein comprise ongoing sessions, wherein each session comprises: (i) administering to the patient an effective amount of Construct 2, wherein the effective amount is about 1×10⁶, 1×10⁷, 1×10¹, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 2 sessions each lasts for 4 weeks, and wherein the 3^{rd} session and subsequent sessions each lasts for 6, 8, 10, 12, 14, 16 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 2 sessions each lasts for 6 weeks, and wherein the 3^{rd} session and subsequent sessions each lasts for 8, 10, 12, 14, 16, 18, 20, or 24 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 2 sessions each lasts for 8 weeks, and wherein the 3^{rd} session and subsequent sessions each lasts for 10, 12, 14, 16, 18, 20, 22, 24, 26, 28, 30, or 32 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 2 sessions each lasts for 10 weeks, and wherein the 3^{rd} session and subsequent sessions each lasts for 12, 14, 16, 18, 20, 22, 24, 26, 28, 30, 32, 34, 36, 38, or 40 weeks. Similarly, the same applies to the methods that comprises administering Construct 1 in (i) and Construct 2 in (ii).

Further provided herein are methods with a modified number of shorter sessions. Accordingly, in some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 3 sessions each lasts for 4 weeks, and wherein the 4^{th} session and subsequent sessions each lasts for 6, 8, 10, 12, 14, 16 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 3 sessions each lasts for 6 weeks, and wherein the 4^{th} session and subsequent sessions each lasts for 8, 10, 12, 14, 16, 18, 20, or 24 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 3 sessions each lasts for 8 weeks, and wherein the 4^{th} session and subsequent sessions each lasts for 10, 12, 14, 16, 18, 20, 22, 24, 26, 28, 30, or 32 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 3 sessions each lasts for 10 weeks, and wherein the 4^{th} session and subsequent sessions each lasts for 12, 14, 16, 18, 20, 22, 24, 26, 28, 30, 32, 34, 36, 38, or 40 weeks. in some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 4 sessions each lasts for 4 weeks, and wherein the 5^{th} session and subsequent sessions each lasts for 6, 8, 10, 12, 14, 16 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 4 sessions each lasts for 6 weeks, and wherein the 5^{th} session and subsequent sessions each lasts for 8, 10, 12, 14, 16, 18, 20, or 24 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 4 sessions each lasts for 8 weeks, and wherein the 5^{th} session and subsequent sessions each lasts for 10, 12, 14, 16, 18, 20, 22, 24, 26, 28, 30, or 32 weeks. In some embodiments, the methods provided herein comprise ongoing sessions, wherein each session comprises: (i) administering to the patient an effective amount of Construct 2, wherein the effective amount is about 1×10⁶, 1×10⁷, 1×10⁸, or 1×10⁹ RCV FFU; and (ii) administering to the patient an effective amount of Construct 1 at a time point around half of the session, wherein the effective amount is about 5×10⁵, 5×10⁶, 5×10⁷, 1×10⁸, or 5×10⁸ RCV FFU, wherein the first 4 sessions each lasts for 10 weeks, and wherein the 5^{th} session and subsequent sessions each lasts for 12, 14, 16, 18, 20, 22, 24, 26, 28, 30, 32, 34, 36, 38, or 40 weeks. Similarly, the same applies to the methods that comprises administering Construct 1 in (i) and Construct 2 in (ii).

An intratumoral injection before the intravenous injections may provide extra efficacy. Therefore, in some embodiments, the methods provided herein comprise an intratumoral injection of Construct 1 followed by ongoing sessions, wherein each session comprises: (i) administering intravenously to the patient an effective amount of Construct 2, wherein the effective amount is about 1×10⁶, 1×10⁷, 1×10⁸, or 1×10⁹ RCV FFU; and (ii) administering intravenously to the patient an effective amount of Construct 1 at a time point around half of the session, wherein the effective amount is about 5×10⁵, 5×10⁶, 5×10⁷, 1×10⁸, or 5×10⁸ RCV FFU. In some embodiments, the methods provided herein comprise an intratumoral injection of Construct 2 followed by ongoing sessions, wherein each session comprises: (i) administering intravenously to the patient an effective amount of Construct 2, wherein the effective amount is about 1×10⁶, 1×10⁷, 1×10⁸, or 1×10⁹ RCV FFU; and (ii) administering intravenously to the patient an effective amount of Construct 1 at a time point around half of the session, wherein the effective amount is about 5×10⁵, 5×10⁶, 5×10⁷, 1×10⁸, or 5×10⁸ RCV FFU. In some embodiments, the methods provided herein comprise an intratumoral injection of Construct 1 followed by ongoing sessions, wherein each session comprises: (i) administering intravenously to the patient an effective amount of Construct 1, wherein the effective amount is about 5×10⁵, 5×10⁶, 5×10⁷, 1×10⁸, or 5×10⁸ RCV FFU; and (ii) administering intravenously to the patient an effective amount of Construct 2 at a time point around half of the session, wherein the effective amount is about 1×10⁶, 1×10⁷, 1×10⁸, or 1×10⁹ RCV FFU. In some embodiments, the methods provided herein comprise an intratumoral injection of Construct 2 followed by ongoing sessions, wherein each session comprises: (i) administering intravenously to the patient an effective amount of Construct 1, wherein the effective amount is about 5×10⁵, 5×10⁶, 5×10⁷, 1×10⁸, or 5×10⁸ RCV FFU; and (ii) administering intravenously to the patient an effective amount of Construct 2 at a time point around half of the session, wherein the effective amount is about 1×10⁶, 1×10⁷, 1×10⁸, or 1×10⁹ RCV FFU.

Further provided herein is a method for treating cancer in a patient in need thereof comprising administering to the patient an effective amount of Construct 1, wherein the effective amount is about 5×10⁵, 5×10⁶, 5×10⁷, 1×10⁸, or 5×10⁸ replication-competent virus focus-forming units (RCV FFU), and wherein Construct 1 is administered intravenously with a frequency of every 3 weeks for 4 cycles followed by ongoing cycles with a frequency of every 6 weeks.

Further provided herein is a method for treating cancer in a patient in need thereof comprising: (i) administering to the patient an effective amount of Construct 1, wherein the effective amount is about 5×10⁵, 5×10⁶, 5×10⁷, 1×10⁸, or 5×10⁸ replication-competent virus focus-forming units (RCV FFU), and wherein Construct 1 is administered intravenously with a frequency of every 3 weeks for 4 cycles followed by ongoing cycles with a frequency of every 6 weeks; and administering to the patient 200 mg of pembrolizumab intravenously with a frequency of every 3 weeks or 400 mg of pembrolizumab intravenously with a frequency of every 6 weeks.

Further provided herein is a method for treating cancer in a patient in need thereof comprising multiple sessions, wherein each session comprises: i. administering to the patient an effective amount of Construct 2 intravenously, wherein the effective amount is about 1×10⁶ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks.

Further provided herein is a method for treating cancer in a patient in need thereof comprising multiple sessions, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks.

Further provided herein is a method for treating cancer in a patient in need thereof comprising multiple sessions, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks.

Further provided herein is a method for treating cancer in a patient in need thereof comprising multiple sessions, wherein each session comprises: administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks.

Further provided herein is a method for treating cancer in a patient in need thereof comprising multiple sessions, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 1×10⁸ RCV FFU, and wherein the first two sessions each lasts for 6 weeks, and the following ongoing sessions each lasts for 12 weeks.

Further provided herein is a method for treating cancer in a patient in need thereof comprising multiple sessions, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁸ RCV FFU, and wherein the first two sessions each lasts for 6 weeks, and the following ongoing sessions each lasts for 12 weeks.

Further provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁶ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Further provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁶ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Further provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Further provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁷ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Further provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 1×10⁸ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Further provided herein is a method for treating cancer in a patient in need thereof comprising (1) multiple sessions of administering Construct 2 and Construct 1, wherein each session comprises: i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁸ RCV FFU, and wherein the first two sessions each lasts for 6 weeks, and the following ongoing sessions each lasts for 12 weeks; and (2) administering to the patient 200 mg of pembrolizumab intravenously at a frequency of every 3 weeks or 400 mg of pembrolizumab intravenously at a frequency of every 6 weeks.

Further provided herein is a method for treating cancer in a patient in need thereof comprising (1) administering intratumorally to the patient an effective amount of Construct 1, wherein the effective amount of Construct 1 is about 5×10⁶ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁶ RCV FFU; and ii administering intravenously to the patient an effective amount of Construct 1 at a time point around half of the session, wherein the effective amount is about 5×10⁶ RCV FFU, and wherein the first two sessions each lasts for 6 weeks, and the following ongoing sessions each lasts for 12 weeks.

Further provided herein is a method for treating cancer in a patient in need thereof comprising (1) administering intratumorally to the patient an effective amount of Construct 1, wherein the effective amount of Construct 1 is about 5×10⁶ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁷ RCV FFU; and ii. administering intravenously to the patient an effective amount of Construct 1 at a time point around half of the session, wherein the effective amount is about 5×10⁶ RCV FFU, and wherein the first two sessions each lasts for 6 weeks, and the following ongoing sessions each lasts for 12 weeks.

Further provided herein is a method for treating cancer in a patient in need thereof comprising (1) administering intratumorally to the patient an effective amount of Construct 1, wherein the effective amount of Construct 1 is about 5×10⁷ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁷ RCV FFU; and ii administering intravenously to the patient an effective amount of Construct 1 at a time point around half of the session, wherein the effective amount is about 5×10⁷ RCV FFU, and wherein the first two sessions each lasts for 6 weeks, and the following ongoing sessions each lasts for 12 weeks.

Further provided herein is a method for treating cancer in a patient in need thereof comprising (1) administering intratumorally to the patient an effective amount of Construct 1, wherein the effective amount of Construct 1 is about 5×10⁷ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁸ RCV FFU; and ii administering intravenously to the patient an effective amount of Construct 1 at a time point around half of the session, wherein the effective amount is about 5×10⁷ RCV FFU, and wherein the first two sessions each lasts for 6 weeks, and the following ongoing sessions each lasts for 12 weeks.

Further provided herein is a method for treating cancer in a patient in need thereof comprising (1) administering intratumorally to the patient an effective amount of Construct 1, wherein the effective amount of Construct 1 is about 1×10⁸ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁸ RCV FFU; and ii administering intravenously to the patient an effective amount of Construct 1 at a time point around half of the session, wherein the effective amount is about 1×10⁸ RCV FFU, and wherein the first two sessions each lasts for 6 weeks, and the following ongoing sessions each lasts for 12 weeks.

Further provided herein is a method for treating cancer in a patient in need thereof comprising (1) administering intratumorally to the patient an effective amount of Construct 1, wherein the effective amount of Construct 1 is about 5×10⁸ replication-competent virus focus-forming units (RCV FFU); and (2) 3 weeks later administering to the patient multiple sessions, wherein each session comprises i. administering intravenously to the patient an effective amount of Construct 2, wherein the effective amount is about 1×10⁸ RCV FFU; and ii administering intravenously to the patient an effective amount of Construct 1 at a time point around half of the session, wherein the effective amount is about 5×10⁸ RCV FFU, and wherein the first two sessions each lasts for 6 weeks, and the following ongoing sessions each lasts for 12 weeks.

Further provided herein is a method for treating cancer in a patient in need thereof comprising administering to the patient an effective amount of Construct 1, wherein the effective amount is about 5×10⁶, 5×10⁷, 5×10⁸, 1×10⁹, or 5×10⁹ replication-competent virus focus-forming units (RCV FFU), and wherein Construct 1 is administered intravenously with a frequency of every 3 weeks for 3 cycles and the method ends after 3 cycles.

Further provided herein is a method for treating cancer in a patient in need thereof comprising 3 sessions, wherein each session comprises i. administering to the patient an effective amount of Construct 2 intravenously, wherein the effective amount is about 1×10⁷ replication-competent virus focus-forming units (RCV FFU); and ii. administering to the patient an effective amount of Construct 1 intravenously at a time point around half of the session, wherein the effective amount is about 5×10⁷ RCV FFU, and wherein each sessions lasts for 6 weeks, and the method ends after 3 sessions.

Further provided herein is a method for treating cancer in a patient in need thereof comprising 3 sessions, wherein each session comprises i. administering to the patient an effective amount of Construct 2 intravenously, wherein the effective amount is about 1×10⁸ replication-competent virus focus-forming units (RCV FFU); and administering to the patient an effective amount of Construct 1 intravenously at a time point around half of the session, wherein the effective amount is about 5×10⁷ RCV FFU, and wherein each sessions lasts for 6 weeks, and the method ends after 3 sessions.

Further provided herein is a method for treating cancer in a patient in need thereof comprising 3 sessions, wherein each session comprises i. administering to the patient an effective amount of Construct 2 intravenously, wherein the effective amount is about 1×10⁸ replication-competent virus focus-forming units (RCV FFU); and ii. administering to the patient an effective amount of Construct 1 intravenously at a time point around half of the session, wherein the effective amount is about 5×10⁸ RCV FFU, and wherein each sessions lasts for 6 weeks, and the method ends after 3 sessions.

Further provided herein is a method for treating cancer in a patient in need thereof comprising 3 sessions, wherein each session comprises i. administering to the patient an effective amount of Construct 2 intravenously, wherein the effective amount is about 1×10⁹ replication-competent virus focus-forming units (RCV FFU); and administering to the patient an effective amount of Construct 1 intravenously at a time point around half of the session, wherein the effective amount is about 5×10⁸ RCV FFU, and wherein each sessions lasts for 6 weeks, and the method ends after 3 sessions.

Further provided herein is a method for treating cancer in a patient in need thereof comprising 3 sessions, wherein each session comprises i. administering to the patient an effective amount of Construct 2 intravenously, wherein the effective amount is about 1×10⁹ replication-competent virus focus-forming units (RCV FFU); and ii. administering to the patient an effective amount of Construct 1 intravenously at a time point around half of the session, wherein the effective amount is about 1×10⁹ RCV FFU, and wherein each sessions lasts for 6 weeks, and the method ends after 3 sessions.

Further provided herein is a method for treating cancer in a patient in need thereof comprising 3 sessions, wherein each session comprises i. administering to the patient an effective amount of Construct 2 intravenously, wherein the effective amount is about 1×10⁹ replication-competent virus focus-forming units (RCV FFU); ii. administering to the patient an effective amount of Construct 1 intravenously at a time point around half of the session, wherein the effective amount is about 5×10⁹ RCV FFU, and wherein each sessions lasts for 6 weeks, and the method ends after 3 sessions.

It is understood that the embodiments described herein for the administration of the engineered replication-competent tri-segmented arenavirus particles derived from a single arenavirus species are applicable for the repeated administration of the engineered replication-competent tri-segmented arenavirus particles derived from different arenavirus species.

In some embodiments, provided herein is pharmaceutical composition comprising an engineered replication-competent tri-segmented arenavirus particle described herein and a pharmaceutically acceptable carrier.

### Nucleic Acids Encoding Dinucleotide Optimized Fusion Protein of Human Papillomavirus Strain 16 (HPV16) E7/E6 for Use in a Method of Treating Cancer

Provided herein are optimized nucleotide sequences that encode the E7/E6 fusion protein of human papillomavirus strain 16 (HPV16). These optimized open reading frames are provided as SEQ ID No: 1 and 2. The sequences are provided in the SEQUENCE TABLE below.

In certain embodiments, these optimized open reading frames are included into an arenaviral genomic segment. SEQ ID No: 3 sets forth a first S segment derived from LCMV with the optimized open reading frame for the E7/E6 fusion protein under control of the 5′ UTR and the open reading frame for the NP protein under control of the 3′ UTR. SEQ ID No: 4 sets forth a second S segment derived from LCMV with the optimized open reading frame for the E7/E6 fusion protein under control of the 5′ UTR and the open reading frame for the GP protein under control of the 3′ UTR. SEQ ID No: 6 sets forth a first S segment derived from PICV with the optimized open reading frame for the E7/E6 fusion protein under control of the 5′ UTR and the open reading frame for the NP protein under control of the 3′ UTR. SEQ ID No: 7 sets forth a second S segment derived from PICV with the optimized open reading frame for the E7/E6 fusion protein under control of the 5′ UTR and the open reading frame for the GP protein under control of the 3′ UTR. These genomic segments (e.g., SEQ ID NOs: 3, 4, and 5; or SEQ ID NOs: 6, 7, and 8) can be incorporated into a viral particle such as S segments 1 and 2 (SEQ ID NOs: 3, 4, 6, 7, respectively), together with an L segment (SEQ ID NOs: 5 and 8, respectively) thereby creating a replicating tri-segmented viral particle encoding the E7/E6 fusion protein.

The nucleotide sequences presented as SEQ ID NOs: 1 to 8 can be RNA or DNA sequences. Once present in a viral particle, these nucleotide sequences can be present as RNA. The DNA sequences shown as SEQ ID NOs: 1-8 can be converted to RNA sequences by replacing the “T” (thymidine) with a “U” (uridine).

The nucleotide sequences provided as SEQ ID NOs: 1-8 can be used in the methods of treatment disclosed herein. In some embodiments, SEQ ID NOs: 3-5 can be used to generate a tri-segmented replication competent viral particle as Construct 1. In some embodiments, SEQ ID NOs: 6-8 can be used to generate a tri-segmented replication competent viral particle as Construct 2.

Provided herein are pharmaceutical compositions comprising engineered replication-competent tri-segmented arenavirus particles comprising SEQ ID NOs: 3-5 and SEQ ID NOs: 6-8, respectively. These pharmaceutical compositions can be used in any of the methods disclosed herein.

Provided herein are expression vectors comprising a nucleotide sequence of SEQ ID NO: 1, 2, 3, 4, 5, 6, 7, or 8. Also provided herein are host cells comprising such an expression vector. Any method known to the skilled artisan can be used to generate a replication competent, tri-segmented viral particle with the genomic segments of SEQ ID NOs: 3 to 5. Any method known to the skilled artisan can be used to generate a replication competent, tri-segmented viral particle with the genomic segments of SEQ ID NOs: 6 to 8. Also provided herein are expression vectors from which any one of the genomic segments of SEQ ID NOs: 3 to 8 can be transcribed.

SEQUENCE TABLE

| SEQ | | |
|---|---|---|
| ID | | |
| NO. | Description | Sequence |
| 1 | Nucleotide | ATGCATGGTGACACCCCCACCCTGCATGAGTACATGCTG |
| | sequence of | GACCTGCAGCCAGAGACCACAGACCTGTATGGCTATGG |
| | HPV16 E7E6 | CCAGCTGAATGACAGCAGTGAGGAAGAGGATGAGATTG |
| | | ATGGGCCAGCAGGCCAGGCAGAACCTGACAGAGCCCAC |
| | | TACAACATTGTCACCTTCTGCTGCAAGTGTGACAGCACC |
| | | CTGAGACTGTGTGTGCAGAGCACCCATGTGGACATCAG |
| | | AACCCTGGAAGACCTGCTGATGGGCACCCTGGGCATTG |
| | | TGGGCCCCATCTGCTCCCAGAAGCCCCACCAGAAAAGA |
| | | ACTGCCATGTTCCAGGACCCCCAGGAGAGGCCCAGAAA |
| | | GCTGCCCCAGCTCTGCACAGAGCTGCAGACCACCATCC |
| | | ATGACATCATCCTGGAATGTGTCTACTGCAAGCAGCAGC |
| | | TGCTGAGGAGAGAGGTGTATGACTTTGCCTTCAGGGAC |
| | | CTGTGCATTGTGTACAGGGATGGCAACCCCTATGCTGTG |
| | | GGGGACAAGTGCCTCAAGTTCTACAGCAAGATCAGTGA |
| | | GTACAGGCACTACTGCTACAGCCTGTATGGCACCACCCT |
| | | GGAACAGCAGTACAACAAGCCCCTGTGTGACCTCCTGA |
| | | TCAGATGCATCAATGGCCAGAAACCCCTCTGCCCTGAG |
| | | GAAAAGCAGAGACACCTGGACAAGAAGCAGAGGTTCC |
| | | ACAACATCAGAGGCAGGTGGACAGGCAGATGCATGAGC |
| | | TGCTGCAGAAGCAGCAGAACCAGAAGAGAGACCCAGCT |
| | | GTGA |
| 2 | Nucleotide | ATGCATGGTGACACCCCCACCCTGCATGAGTACATGCTG |
| | sequence of | GACCTGCAGCCAGAGACCACAGACCTGTATGGCTATGG |
| | HPV16 E7E6 | CCAGCTGAATGACAGCAGTGAGGAAGAGGATGAGATTG |
| | | ATGGGCCAGCAGGCCAGGCAGAACCTGACAGAGCCCAC |
| | | TACAACATTGTCACCTTCTGCTGCAAGTGTGACAGCACC |
| | | CTGAGACTGTGTGTGCAGAGCACCCATGTGGACATCAG |
| | | AACCCTGGAAGACCTGCTGATGGGCACCCTGGGCATTG |
| | | TGGGCCCCATCTGCTCCCAGAAGCCCCACCAGAAAAGA |
| | | ACTGCCATGTTCCAGGACCCCCAGGAGAGGCCCAGAAA |
| | | GCTGCCCCAGCTCTGCACAGAGCTGCAGACCACCATCC |
| | | ATGACATCATCCTGGAATGTGTCTACTGCAAGCAGCAGC |
| | | TGCTGAGGAGAGAGGTGTATGACTTTGCCTTCAGGGAC |
| | | CTGTGCATTGTGTACAGGGATGGCAACCCCTATGCTGTG |
| | | GGGGACAAGTGCCTCAAGTTCTACAGTAAGATCAGTGA |
| | | GTACAGGCACTACTGCTACAGCCTGTATGGCACCACCCT |
| | | GGAACAGCAGTACAACAAGCCCCTGTGTGACCTCCTGA |
| | | TCAGATGCATCAATGGCCAGAAACCCCTCTGCCCTGAG |
| | | GAAAAGCAGAGACACCTGGACAAGAAGCAGAGGTTCC |
| | | ACAACATCAGAGGCAGGTGGACAGGCAGATGCATGAGC |
| | | TGCTGCAGAAGCAGCAGAACCAGAAGAGAGACCCAGCT |
| | | GTGA |
| 3 | Nucleotide | GCGCACCGGGGATCCTAGGCTTTTTGGATTGCGCTTTCC |
| | sequence of | TCTAGATCAACTGGGTGTCAGGCCCTATCCTACAGAAGG |
| | LCMV HPV16 | ATGCATGGTGACACCCCCACCCTGCATGAGTACATGCTG |
| | E7E6-NP-S- | GACCTGCAGCCAGAGACCACAGACCTGTATGGCTATGG |
| | segment 1 | CCAGCTGAATGACAGCAGTGAGGAAGAGGATGAGATTG |
| | (The genomic | ATGGGCCAGCAGGCCAGGCAGAACCTGACAGAGCCCAC |
| | segment is RNA, | TACAACATTGTCACCTTCTGCTGCAAGTGTGACAGCACC |
| | the sequence in | CTGAGACTGTGTGTGCAGAGCACCCATGTGGACATCAG |
| | SEQ ID NO: 3 is | AACCCTGGAAGACCTGCTGATGGGCACCCTGGGCATTG |
| | shown for DNA; | TGGGCCCCATCTGCTCCCAGAAGCCCCACCAGAAAAGA |
| | however, | ACTGCCATGTTCCAGGACCCCCAGGAGAGGCCCAGAAA |
| | exchanging all | GCTGCCCCAGCTCTGCACAGAGCTGCAGACCACCATCC |
| | thymidines (“T”) | ATGACATCATCCTGGAATGTGTCTACTGCAAGCAGCAGC |
| | in SEQ ID NO: 3 | TGCTGAGGAGAGAGGTGTATGACTTTGCCTTCAGGGAC |
| | for uridines (“U”) | CTGTGCATTGTGTACAGGGATGGCAACCCCTATGCTGTG |
| | provides the RNA | GGGGACAAGTGCCTCAAGTTCTACAGCAAGATCAGTGA |
| | sequence.) | GTACAGGCACTACTGCTACAGCCTGTATGGCACCACCCT |
| | | GGAACAGCAGTACAACAAGCCCCTGTGTGACCTCCTGA |
| | | TCAGATGCATCAATGGCCAGAAACCCCTCTGCCCTGAG |
| | | GAAAAGCAGAGACACCTGGACAAGAAGCAGAGGTTCC |
| | | ACAACATCAGAGGCAGGTGGACAGGCAGATGCATGAGC |
| | | TGCTGCAGAAGCAGCAGAACCAGAAGAGAGACCCAGCT |
| | | GTGAAGAACAGCGCCTCCCTGACTCTCCACCTCGAAAG |
| | | AGGTGGAGAGTCAGGGAGGCCCAGAGGGTCTTAGAGTG |
| | | TCACAACATTTGGGCCTCTAAAAATTAGGTCATGTGGCA |
| | | GAATGTTGTGAACAGTTTTCAGATCTGGGAGCCTTGCTT |
| | | TGGAGGCGCTTTCAAAAATGATGCAGTCCATGAGTGCA |
| | | CAGTGCGGGGTGATCTCTTTCTTCTTTTTGTCCCTTACTA |
| | | TTCCAGTATGCATCTTACACAACCAGCCATATTTGTCCC |
| | | ACACTTTATCTTCATACTCCCTCGAAGCTTCCCTGGTCAT |
| | | TTCAACATCGATAAGCTTAATGTCCTTCCTATTTTGTGA |
| | | GTCCAGAAGCTTTCTGATGTCATCGGAGCCTTGACAGCT |
| | | TAGAACCATCCCCTGCGGAAGAGCACCTATAACTGACG |
| | | AGGTCAACCCGGGTTGCGCATTGAAGAGGTCGGCAAGA |
| | | TCCATGCCGTGTGAGTACTTGGAATCTTGCTTGAATTGT |
| | | TTTTGATCAACGGGTTCCCTGTAAAAGTGTATGAACTGC |
| | | CCGTTCTGTGGTTGGAAAATTGCTATTTCCACTGGATCA |
| | | TTAAATCTACCCTCAATGTCAATCCATGTAGGAGCGTTG |
| | | GGGTCAATTCCTCCCATGAGGTCTTTTAAAAGCATTGTC |
| | | TGGCTGTAGCTTAAGCCCACCTGAGGTGGACCTGCTGCT |
| | | CCAGGCGCTGGCCTGGGTGAGTTGACTGCAGGTTTCTCG |
| | | CTTGTGAGATCAATTGTTGTGTTTTCCCATGCTCTCCCCA |
| | | CAATCGATGTTCTACAAGCTATGTATGGCCATCCTTCAC |
| | | CTGAAAGGCAAACTTTATAGAGGATGTTTTCATAAGGGT |
| | | TCCTGTCCCCAACTTGGTCTGAAACAAACATGTTGAGTT |
| | | TTCTCTTGGCCCCGAGAACTGCCTTCAAGAGATCCTCGC |
| | | TGTTGCTTGGCTTGATCAAAATTGACTCTAACATGTTAC |
| | | CCCCATCCAACAGGGCTGCCCCTGCCTTCACGGCAGCAC |
| | | CAAGACTAAAGTTATAGCCAGAAATGTTGATGCTGGAC |
| | | TGCTGTTCAGTGATGACCCCCAGAACTGGGTGCTTGTCT |
| | | TTCAGCCTTTCAAGATCATTAAGATTTGGATACTTGACT |
| | | GTGTAAAGCAAGCCAAGGTCTGTGAGCGCTTGTACAAC |
| | | GTCATTGAGCGGAGTCTGTGACTGTTTGGCCATACAAGC |
| | | CATAGTTAGACTTGGCATTGTGCCAAATTGATTGTTCAA |
| | | AAGTGATGAGTCTTTCACATCCCAAACTCTTACCACACC |
| | | ACTTGCACCCTGCTGAGGCTTTCTCATCCCAACTATCTG |
| | | TAGGATCTGAGATCTTTGGTCTAGTTGCTGTGTTGTTAA |
| | | GTTCCCCATATATACCCCTGAAGCCTGGGGCCTTTCAGA |
| | | CCTCATGATCTTGGCCTTCAGCTTCTCAAGGTCAGCCGC |
| | | AAGAGACATCAGTTCTTCTGCACTGAGCCTCCCCACTTT |
| | | CAAAACATTCTTCTTTGATGTTGACTTTAAATCCACAAG |
| | | AGAATGTACAGTCTGGTTGAGACTTCTGAGTCTCTGTAG |
| | | GTCTTTGTCATCTCTCTTTTCCTTCCTCATGATCCTCTGA |
| | | ACATTGCTGACCTCAGAGAAGTCCAACCCATTCAGAAG |
| | | GTTGGTTGCATCCTTAATGACAGCAGCCTTCACATCTGA |
| | | TGTGAAGCTCTGCAATTCTCTTCTCAATGCTTGCGTCCAT |
| | | TGGAAGCTCTTAACTTCCTTAGACAAGGACATCTTGTTG |
| | | CTCAATGGTTTCTCAAGACAAATGCGCAATCAAATGCCT |
| | | AGGATCCACTGTGCG |
| 4 | Nucleotide | GCGCACCGGGGATCCTAGGCTTTTTGGATTGCGCTTTCC |
| | sequence of | TCTAGATCAACTGGGTGTCAGGCCCTATCCTACAGAAGG |
| | LCMV HPV16 | ATGCATGGTGACACCCCCACCCTGCATGAGTACATGCTG |
| | E7E6-GP-S- | GACCTGCAGCCAGAGACCACAGACCTGTATGGCTATGG |
| | segment 2 | CCAGCTGAATGACAGCAGTGAGGAAGAGGATGAGATTG |
| | (The genomic | ATGGGCCAGCAGGCCAGGCAGAACCTGACAGAGCCCAC |
| | segment is RNA, | TACAACATTGTCACCTTCTGCTGCAAGTGTGACAGCACC |
| | the sequence in | CTGAGACTGTGTGTGCAGAGCACCCATGTGGACATCAG |
| | SEQ ID NO: 4 is | AACCCTGGAAGACCTGCTGATGGGCACCCTGGGCATTG |
| | shown for DNA; | TGGGCCCCATCTGCTCCCAGAAGCCCCACCAGAAAAGA |
| | however, | ACTGCCATGTTCCAGGACCCCCAGGAGAGGCCCAGAAA |
| | exchanging all | GCTGCCCCAGCTCTGCACAGAGCTGCAGACCACCATCC |
| | thymidines (“T”) | ATGACATCATCCTGGAATGTGTCTACTGCAAGCAGCAGC |
| | in SEQ ID NO: 4 | TGCTGAGGAGAGAGGTGTATGACTTTGCCTTCAGGGAC |
| | for uridines (“U”) | CTGTGCATTGTGTACAGGGATGGCAACCCCTATGCTGTG |
| | provides the RNA | GGGGACAAGTGCCTCAAGTTCTACAGTAAGATCAGTGA |
| | sequence.) | GTACAGGCACTACTGCTACAGCCTGTATGGCACCACCCT |
| | | GGAACAGCAGTACAACAAGCCCCTGTGTGACCTCCTGA |
| | | TCAGATGCATCAATGGCCAGAAACCCCTCTGCCCTGAG |
| | | GAAAAGCAGAGACACCTGGACAAGAAGCAGAGGTTCC |
| | | ACAACATCAGAGGCAGGTGGACAGGCAGATGCATGAGC |
| | | TGCTGCAGAAGCAGCAGAACCAGAAGAGAGACCCAGCT |
| | | GTGAAGAACAGCGCCTCCCTGACTCTCCACCTCGAAAG |
| | | AGGTGGAGAGTCAGGGAGGCCCAGAGGGTCTCAGCGTC |
| | | TTTTCCAGATAGTTTTTACACCAGGCACCTTGAATGCAC |
| | | CACAACTACAGATCCCCTTGTTGGTCAAGCGGTGTGGCT |
| | | TTGGACATGAACCGCCCTTTATGTGTCTATGTGTTGGTA |
| | | TCTTCACAAGATGCAGAAAGATGCTGATTAGATATGCTG |
| | | ATGTTGAAAACATCAAAAGATCCATTAAGGCTAAAGGA |
| | | GTACTCCCTTGTCTTTTTATGTAGTCCTTCCTCAACATCT |
| | | CTGTGATCATGTTATCTGCTTCTTGTTCGATTTGATCACT |
| | | AAAGTGGGTCTCATTCAAGTAGGAGCCATTAGTGACAA |
| | | GCCAGCACTTGGGTACACTAGTCTCACCAGTCTTAGCAT |
| | | GTTCCAGATACCAGAACTTTGAGTAATTACAGTATGGTA |
| | | CCCCCATTAGATCTCTTAGATGATTCCTCATCAACAGCT |
| | | GATCGGAAATCAGAGAATTTACTGTTGTTTTGAATACAT |
| | | GCAAGGCAGACTCTACATCTTGCTTGAACTTACTCAGGG |
| | | CGGCCTTGTTGTAATCAATTAGTCGTAGCATGTCACAGA |
| | | ACTCTTCATCATGATTGACATTACATTTTGCAACAGCTG |
| | | TATTCCCAAAACATTTGAGCTCTGCAGCAAGGATCATCC |
| | | ATTTGGTCAGGCAATAACCACCTGGATTTTCTACTCCTG |
| | | AGGAGTCTGACAGGGTCCAGGTGAATGTGCCTGCAAGT |
| | | CTCCTAGTGAGAAACTTTGTCTTTTCCTGAGCAAAGAGG |
| | | ATTCTAGACATCCCAAAAGGGCCTGCATATCTACAGTGG |
| | | TTTTCCCAAGTCCTGTTTTGTATGATTAGGTACTGATAGC |
| | | TTGTTTGGCTGCACCAAGTGGTCTTGCCATCTGAACCTG |
| | | CCCAGCCCCAGCCACTTCTCATGTATTTTCCTCCAAAGG |
| | | CAGTTCTAAACATGTCCAAGACTCTACCTCTGAAAGTCC |
| | | TACACTGGCTTATAGCGCTCTGTGGGTCCGAAAATGACA |
| | | AGTTGTATTGAATGGTGATGCCATTGTTAAAATCACAAG |
| | | ACACTGCTTTGTGGTTGGAATTCCCTCTAATACTGAGGT |
| | | GCAGACTCGAGACTATACTCATGAGTGTATGGTCAAAA |
| | | GTCTTTTTGTTGAAAGCGGAGGTTAAGTTGCAAAAATTG |
| | | TGATTAAGGATGGAGTCGTTAGTGAAAGTTAGCTCCAGT |
| | | CCAGAGCTTCCCATACTGATGTAGTGATGAGAGTTGTTG |
| | | GCTGAGCACGCATTGGGCATCGTCAGATTTAAGTGAGA |
| | | CATATCAAACTCCACTGATTTGAACTGGTAAACCCCTTT |
| | | ATAGATGTCGGGACCATTAAGGCCGTACATGCCACAGG |
| | | ACCTACCAGCCAAAAAAAGGAAGCTGACCAGTGCTAAT |
| | | ATCCCACAGGTGGCGAAATTGTACACAGCTTTGATGCTC |
| | | GTGATTATAATGAGCACAATAATGACAATGTTGATGAC |
| | | CTCATCAATGATGTGAGGCAAAGCCTCAAACATTGTCAC |
| | | AATCTGACCCATCTTGTTGCTCAATGGTTTCTCAAGACA |
| | | AATGCGCAATCAAATGCCTAGGATCCACTGTGCG |
| 5 | Nucleotide | GCGCACCGGGGATCCTAGGCGTTTAGTTGCGCTGTTTGG |
| | sequence of | TTGCACAACTTTCTTCGTGAGGCTGTCAGAAGTGGACCT |
| | LCMV L- | GGCTGATAGCGATGGGTCAAGGCAAGTCCAGAGAGGAG |
| | segment | AAAGGCACCAATAGTACAAACAGGGCCGAAATCCTACC |
| | (The genomic | AGATACCACCTATCTTGGCCCTTTAAGCTGCAAATCTTG |
| | segment is RNA, | CTGGCAGAAATTTGACAGCTTGGTAAGATGCCATGACC |
| | the sequence in | ACTACCTTTGCAGGCACTGTTTAAACCTTCTGCTGTCAG |
| | SEQ ID NO: 5 is | TATCCGACAGGTGTCCTCTTTGTAAATATCCATTACCAA |
| | shown for DNA; | CCAGATTGAAGATATCAACAGCCCCAAGCTCTCCACCTC |
| | however, | CCTACGAAGAGTAACACCGTCCGGCCCCGGCCCCGACA |
| | exchanging all | AACAGCCCAGCACAAGGGAACCGCACGTCACCCAACGC |
| | thymidines (“T”) | ACACAGACACAGCACCCAACACAGAACACGCACACACA |
| | in SEQ ID NO: 6 | CACACACACACACCCACACGCACGCGCCCCCACCACCG |
| | for uridines (“U”) | GGGGGCGCCCCCCCCCGGGGGGCGGCCCCCCGGGAGCC |
| | provides the RNA | CGGGCGGAGCCCCACGGAGATGCCCATCAGTCGGTGTC |
| | sequence.) | CTCGGCCACCGACCCGCCTAGCCAATCGTCGCAGGACCT |
| | | CCCCTTGAGTCTAAACCTGCCCCCCACTGTTTCATACAT |
| | | CAAAGTGCTCCTAGATTTGCTAAAACAAAGTCTGCAATC |
| | | CTTAAAGGCGAACCAGTCTGGCAAAAGCGACAGTGGAA |
| | | TCAGCAGAATAGATCTGTCTATACATAGTTCCTGGAGGA |
| | | TTACACTTATCTCTGAACCCAACAAATGTTCACCAGTTC |
| | | TGAATCGATGCAGGAAGAGGTTCCCAAGGACATCACTA |
| | | ATCTTTTCATAGCCCTCAAGTCCTGCTAGAAAGACTTTC |
| | | ATGTCCTTGGTCTCCAGCTTCACAATGATATTTTGGACA |
| | | AGGTTTCTTCCTTCAAAAAGGGCACCCATCTTTACAGTC |
| | | AGTGGCACAGGCTCCCACTCAGGTCCAACTCTCTCAAAG |
| | | TCAATAGATCTAATCCCATCCAGTATTCTTTTGGAGCCC |
| | | AACAACTCAAGCTCAAGAGAATCACCAAGTATCAAGGG |
| | | ATCTTCCATGTAATCCTCAAACTCTTCAGATCTGATATC |
| | | AAAGACACCATCGTTCACCTTGAAGACAGAGTCTGTCCT |
| | | CAGTAAGTGGAGGCATTCATCCAACATTCTTCTATCTAT |
| | | CTCACCCTTAAAGAGGTGAGAGCATGATAAAAGTTCAG |
| | | CCACACCTGGATTCTGTAATTGGCACCTAACCAAGAATA |
| | | TCAATGAAAATTTCCTTAAACAGTCAGTATTATTCTGAT |
| | | TGTGCGTAAAGTCCACTGAAATTGAAAACTCCAATAC |
| | | CCCTTTTGTGTAGTTGAGCATGTAGTCCCACAGATCCTT |
| | | TAAGGATTTAAATGCCTTTGGGTTTGTCAGGCCCTGCCT |
| | | AATCAACATGGCAGCATTACACACAACATCTCCCATTCG |
| | | GTAAGAGAACCACCCAAAACCAAACTGCAAATCATTCC |
| | | TAAACATAGGCCTCTCCACATTTTTGTTCACCACCTTTG |
| | | AGACAAATGATTGAAAGGGGCCCAGTGCCTCAGCACCA |
| | | TCTTCAGATGGCATCATTTCTTTATGAGGGAACCATGA |
| | | AAAATTGCCTAATGTCCTGGTTGTTGCAACAAATTCTCG |
| | | AACAAATGATTCAAAATACACCTGTTTTAAGAAGTTCTT |
| | | GCAGACATCCCTCGTGCTAACAACAAATTCATCAACCA |
| | | GACTGGAGTCAGATCGCTGATGAGAATTGGCAAGGTCA |
| | | GAAAACAGAACAGTGTAATGTTCATCCCTTTTCCACTTA |
| | | ACAACATGAGAAATGAGTGACAAGGATTCTGAGTTAAT |
| | | ATCAATTAAAACACAGAGGTCAAGGAATTTAATTCTGG |
| | | GACTCCACCTCATGTTTTTTGAGCTCATGTCAGACATAA |
| | | ATGGAAGAAGCTGATCCTCAAAGATCTTGGGATATAGC |
| | | CGCCTCACAGATTGAATCACTTGGTTCAAATTCACTTTG |
| | | TCCTCCAGTAGCCTTGAGCTCTCAGGCTTTCTTGCTACAT |
| | | AATCACATGGGTTTAAGTGCTTAAGAGTTAGGTTCTCAC |
| | | TGTTATTCTTCCCTTTGGTCGGTTCTGCTAGGACCCAAAC |
| | | ACCCAACTCAAAAGAGTTGCTCAATGAAATACAAAT |
| | | GTAGTCCCAAAGAAGAGGCCTTAAAAGGCATATATGAT |
| | | CACGGTGGGCTTCTGGATGAGACTGTTTGTCACAAATGT |
| | | ACAGCGTTATACCATCCCGATTGCAAACTCTTGTCACAT |
| | | GATCATCTGTGGTTAGATCCTCAAGCAGCTTTTTGATAT |
| | | ACAGATTTTCCCTATTTTTGTTTCTCACACACCTGCTTCC |
| | | TAGAGTTTTGCAAAGGCCTATAAAGCCAGATGAGATAC |
| | | AACTCTGGAAAGCTGACTTGTTGATTGCTTCTGACAG |
| | | CAGCTTCTGTGCACCCCTTGTGAATTTACTACAAAGTTT |
| | | GTTCTGGAGTGTCTTGATCAATGATGGGATTCTTTCCTCT |
| | | TGGAAAGTCATCACTGATGGATAAACCACCTTTTGTCTT |
| | | AAAACCATCCTTAATGGGAACATTTCATTCAAATTCAAC |
| | | CAGTTAACATCTGCTAACTGATTCAGATCTTCTTCAAGA |
| | | CCGAGGAGGTCTCCCAATTGAAGAATGGCCTCCTTTTTA |
| | | TCTCTGTTAAATAGGTCTAAGAAAAATTCTTCATTAAAT |
| | | TCACCATTTTTGAGCTTATGATGCAGTTTCCTTACAAGCT |
| | | TTCTTACAACCTTTGTTTCATTAGGACACAGTTCCTCAAT |
| | | GAGTCTTTGTATTCTGTAACCTCTAGAACCATCCAGCCA |
| | | ATCTTTCACATCAGTGTTGGTATTCAGTAGAAATGGATC |
| | | CAAAGGGAAATTGGCATACTTTAGGAGGTCCAGTGTTCT |
| | | CCTTTGGATACTATTAACTAGGGAGACTGGGACGCCATT |
| | | TGCGATGGCTTGATCTGCAATTGTATCTATTGTTTCACA |
| | | AAGTTGATGTGGCTCTTTACACTTGACATTGTGTAGCGC |
| | | TGCAGATACAAACTTTGTGAGAAGAGGGACTTCCTCCCC |
| | | CCATACATAGAATCTAGATTTAAATTCTGCAGCGAACCT |
| | | CCCAGCCACACTTTTTGGGCTGATAAATTTGTTTAACAA |
| | | GCCGCTCAGATGAGATTGGAATTCCAACAGGACAAGGA |
| | | CTTCCTCCGGATCACTTACAACCAGGTCACTCAGCCTCC |
| | | TATCAAATAAAGTGATCTGATCATCACTTGATGTGTAAG |
| | | CCTCTGGTCTTTCGCCAAAGATAACACCAATGCAGTAGT |
| | | TGATGAACCTCTCGCTAAGCAAACCATAGAAGTCAGAA |
| | | GCATTATGCAAGATTCCCTGCCCCATATCAATAAGGCTG |
| | | GATATATGGGATGGCACTATCCCCATTTCAAAATATTGT |
| | | CTGAAAATTCTCTCAGTAACAGTTGTTTCTGAACCCCTG |
| | | AGAAGTTTTAGCTTCGACTTGACATATGATTTCATCATT |
| | | GCATTCACAACAGGAAAGGGGACCTCGACAAGCTTATG |
| | | CATGTGCCAAGTTAACAAAGTGCTAACATGATCTTTCCC |
| | | GGAACGCACATACTGGTCATCACCTAGTTTGAGATTTTG |
| | | TAGAAACATTAAGAACAAAAATGGGCACATCATTGGTC |
| | | CCCATTTGCTGTGATCCATACTATAGTTTAAGAACCCTT |
| | | CCCGCACATTGATAGTCATTGACAAGATTGCATTTTCAA |
| | | ATTCCTTATCATTGTTTAAACAGGAGCCTGAAAAGAAAC |
| | | TTGAAAAAGACTCAAAATAATCTTCTATTAACCTTGTGA |
| | | ACATTTTTGTCCTCAAATCTCCAATATAGAGTTCTCTATT |
| | | TCCCCCAACCTGCTCTTTATAAGATAGTGCAAATTTCAG |
| | | CCTTCCAGAGTCAGGACCTACTGAGGTGTATGATGTTGG |
| | | TGATTCTTCTGAGTAGAAGCACAGATTTTTCAAAGCAGC |
| | | ACTCATACATTGTGTCAACGACAGAGCTTTACTAAGGGA |
| | | CTCAGAATTACTTTCCCTCTCACTGATTCTCACGTCTTCT |
| | | TCCAGTTTGTCCCAGTCAAATTTGAAATTCAAGCCTTGC |
| | | CTTTGCATATGCCTGTATTTCCCTGAGTACGCATTTGCAT |
| | | TCATTTGCAACAGAATCATCTTCATGCAAGAAAACCAAT |
| | | CATTCTCAGAAAAGAACTTTCTACAAAGGTTTTTTGCCA |
| | | TCTCATCGAGGCCACACTGATCTTTAATGACTGAGGTGA |
| | | AATACAAAGGTGACAGCTCTGTGGAACCCTCAACAGCC |
| | | TCACAGATAAATTTCATGTCATCATTGGTTAGACATGAT |
| | | GGGTCAAAGTCTTCTACTAAATGGAAAGATATTTCTGAC |
| | | AAGATAACTTTTCTTAAGTGAGCCATCTTCCCTGTTAGA |
| | | ATAAGCTGTAAATGATGTAGTCCTTTTGTATTTGTAAGT |
| | | TTTTCTCCATCTCCTTTGTCATTGGCCCTCCTACCTCTTCT |
| | | GTACCGTGCTATTGTGGTGTTGACCTTTTCTTCGAGACT |
| | | TTTGAAGAAGCTTGTCTCTTCTTCTCCATCAAAACATATT |
| | | TCTGCCAGGTTGTCTTCCGATCTCCCTGTCTCTTCTCCCT |
| | | TGGAACCGATGACCAATCTAGAGACTAACTTGGAAACT |
| | | TTATATTCATAGTCTGAGTGGCTCAACTTATACTTTTGTT |
| | | TTCTTACGAAACTCTCCGTAATTTGACTCACAGCACTAA |
| | | CAAGCAATTTGTTAAAGTCATATTCCAGAAGTCGTTCTC |
| | | CATTTAGATGCTTATTAACCACCACACTTTTGTTACTAG |
| | | CAAGATCTAATGCTGTCGCACATCCAGAGTTAGTCATGG |
| | | GATCTAGGCTGTTTAGCTTCTTCTCTCCTTTGAAAATTAA |
| | | AGTGCCGTTGTTAAATGAAGACACCATTAGGCTAAAGG |
| | | CTTCCAGATTAACACCTGGAGTTGTATGCTGACAGTCAA |
| | | TTTCTTTACTAGTGAATCTCTTCATTTGCTCATAGAACAC |
| | | ACATTCTTCCTCAGGAGTGATTGCTTCCTTGGGGTTGAC |
| | | AAAAAAACCAAATTGACTTTTGGGCTCAAAGAACTTTTC |
| | | AAAACATTTTATCTGATCTGTTAGCCTGTCAGGGGTCTC |
| | | CTTTGTGATCAAATGACACAGGTATGACACATTCAACAT |
| | | AAATTTAAATTTTGCACTCAACAACACCTTCTCACCAGT |
| | | ACCAAAAATAGTTTTTATTAGGAATCTAAGCAGCTTATA |
| | | CACCACCTTCTCAGCAGGTGTGATCAGATCCTCCCTCAA |
| | | CTTATCCATTAATGATGTAGATGAAAAATCTGACACTAT |
| | | TGCCATCACCAAATATCTGACACTCTGTACCTGCTTTTG |
| | | ATTTCTCTTTGTTGGGTTGGTGAGCATTAGCAACAATAG |
| | | GGTCCTCAGTGCAACCTCAATGTCGGTGAGACAGTCTTT |
| | | CAAATCAGGACATGATCTAATCCATGAAATCATGATGTC |
| | | TATCATATTGTATAAGACCTCATCTGAAAAAATTGGTAA |
| | | AAAGAACCTTTTAGGATCTGCATAGAAGGAAATTAAAT |
| | | GACCATCCGGGCCTTGTATGGAGTAGCACCTTGAAGATT |
| | | CTCCAGTCTTCTGGTATAATAGGTGGTATTCTTCAGAGT |
| | | CCAGTTTTATTACTTGGCAAAACACTTCTTTGCATTCTAC |
| | | CACTTGATATCTCACAGACCCTATTTGATTTTGCCTTAGT |
| | | CTAGCAACTGAGCTAGTTTTCATACTGTTTGTTAAGGCC |
| | | AGACAAACAGATGATAATCTTCTCAGGCTCTGTATGTTC |
| | | TTCAGCTGCTCTGTGCTGGGTTGGAAATTGTAATCTTCA |
| | | AACTTCGTATAATACATTATCGGGTGAGCTCCAATTTTC |
| | | ATAAAGTTCTCAAATTCAGTGAATGGTATGTGGCATTCT |
| | | TGCTCAAGGTGTTCAGACAGTCCGTAATGCTCGAAACTC |
| | | AGTCCCACCACTAACAGGCATTTTTGAATTTTTGCAATG |
| | | AACTCACTAATAGATGCCCTAAACAATTCCTCAAAAGA |
| | | CACCTTTCTAAACACCTTTGACTTTTTTCTATTCCTCAAA |
| | | AGTCTAATGAACTCCTCTTTAGTGCTGTGAAAGCTTACC |
| | | AGCCTATCATTCACACTACTATAGCAACAACCCACCCAG |
| | | TGTTTATCATTTTTTAACCCTTTGAATTTCGACTGTTTTA |
| | | TCAATGAGGAAAGACACAAAACATCCAGATTTAACAAC |
| | | TGTCTCCTTCTAGTATTCAACAGTTTCAAACTCTTGACTT |
| | | TGTTTAACATAGAGAGGAGCCTCTCATATTCAGTGCTAG |
| | | TCTCACTTCCCCTTTCGTGCCCATGGGTCTCTGCAGTTAT |
| | | GAATCTCATCAAAGGACAGGATTCGACTGCCTCCCTGCT |
| | | TAATGTTAAGATATCATCACTATCAGCAAGGTTTTCATA |
| | | GAGCTCAGAGAATTCCTTGATCAAGCCTTCAGGGTTTAC |
| | | TTTCTGAAAGTTTCTCTTTAATTTCCCACTTTCTAAATCT |
| | | CTTCTAAACCTGCTGAAAAGAGAGTTTATTCCAAAAACC |
| | | ACATCATCACAGCTCATGTTGGGGTTGATGCCTTCGTGG |
| | | CACATCCTCATAATTTCATCATTGTGAGTTGACCTCGCA |
| | | TCTTTCAGAATTTTCATAGAGTCCATACCGGAGCGCTTG |
| | | TCGATAGTAGTCTTCAGGGACTCACAGAGTCTAAAATAT |
| | | TCAGACTCTTCAAAGACTTTCTCATTTTGGTTAGAATAC |
| | | TCCAAAAGTTTGAATAAAAGGTCTCTAAATTTGAAGTTT |
| | | GCCCACTCTGGCATAAAACTATTATCATAATCACAACGA |
| | | CCATCTACTATTGGAACTAATGTGACACCCGCAACAGCA |
| | | AGGTCTTCCCTGATGCATGCCAATTTGTTAGTGTCCTCT |
| | | ATAAATTTCTTCTCAAAACTGGCTGGAGTGCTCCTAACA |
| | | AAACACTCAAGAAGAATGAGAGAATTGTCTATCAGCTT |
| | | GTAACCATCAGGAATGATAAGTGGTAGTCCTGGGCATA |
| | | CAATTCCAGACTCCACCAAAATTGTTTCCACAGACTTAT |
| | | CGTCGTGGTTGTGTGTGCAGCCACTCTTGTCTGCACTGT |
| | | CTATTTCAATGCAGCGTGACAGCAACTTGAGTCCCTCAA |
| | | TCAGAACCATTCTGGGTTCCCTTTGTCCCAGAAAGTTGA |
| | | GTTTCTGCCTTGACAACCTCTCATCCTGTTCTATATAGTT |
| | | TAAACATAACTCTCTCAATTCTGAGATGATTTCATCCAT |
| | | TGCGCATCAAAAAGCCTAGGATCCTCGGTGCG |
| 6 | Nucleotide | GCGCACCGGGGATCCTAGGCATACCTTGGACGCGCATA |
| | sequence of PICV | TTACTTGATCAAAGATGCATGGTGACACCCCCACCCTGC |
| | HPV16 E7E6- | ATGAGTACATGCTGGACCTGCAGCCAGAGACCACAGAC |
| | NP-S-segment 1 | CTGTATGGCTATGGCCAGCTGAATGACAGCAGTGAGGA |
| | (The genomic | AGAGGATGAGATTGATGGGCCAGCAGGCCAGGCAGAAC |
| | segment is RNA, | CTGACAGAGCCCACTACAACATTGTCACCTTCTGCTGCA |
| | the sequence in | AGTGTGACAGCACCCTGAGACTGTGTGTGCAGAGCACC |
| | SEQ ID NO: 6 is | CATGTGGACATCAGAACCCTGGAAGACCTGCTGATGGG |
| | shown for DNA; | CACCCTGGGCATTGTGGGCCCCATCTGCTCCCAGAAGCC |
| | however, | CCACCAGAAAAGAACTGCCATGTTCCAGGACCCCCAGG |
| | exchanging all | AGAGGCCCAGAAAGCTGCCCCAGCTCTGCACAGAGCTG |
| | thymidines (“T”) | CAGACCACCATCCATGACATCATCCTGGAATGTGTCTAC |
| | in SEQ ID NO: 6 | TGCAAGCAGCAGCTGCTGAGGAGAGAGGTGTATGACTT |
| | for uridines (“U”) | TGCCTTCAGGGACCTGTGCATTGTGTACAGGGATGGCAA |
| | provides the RNA | CCCCTATGCTGTGGGGGACAAGTGCCTCAAGTTCTACAG |
| | sequence.) | CAAGATCAGTGAGTACAGGCACTACTGCTACAGCCTGT |
| | | ATGGCACCACCCTGGAACAGCAGTACAACAAGCCCCTG |
| | | TGTGACCTCCTGATCAGATGCATCAATGGCCAGAAACCC |
| | | CTCTGCCCTGAGGAAAAGCAGAGACACCTGGACAAGAA |
| | | GCAGAGGTTCCACAACATCAGAGGCAGGTGGACAGGCA |
| | | GATGCATGAGCTGCTGCAGAAGCAGCAGAACCAGAAGA |
| | | GAGACCCAGCTGTGAGCCCTAGCCTCGACATGGGCCTC |
| | | GACGTCACTCCCCAATAGGGGAGTGACGTCGAGGCCTC |
| | | TGAGGACTTGAGCTCAGAGGTTGATCAGATCTGTGTTGT |
| | | TCCTGTACAGCGTGTCAATAGGCAAGCATCTCATCGGCT |
| | | TCTGGTCCCTAACCCAGCCTGTCACTGTTGCATCAAACA |
| | | TGATGGTATCAAGCAATGCACAGTGAGGATTCGCAGTG |
| | | GTTTGTGCAGCCCCCTTCTTCTTCTTCTTTATGACCAAAC |
| | | CTTTATGTTTGGTGCAGAGTAGATTGTATCTCTCCCAGA |
| | | TCTCATCCTCAAAGGTGCGTGCTTGCTCGGCACTGAGTT |
| | | TCACGTCAAGCACTTTTAAGTCTCTTCTCCCATGCATTTC |
| | | GAACAAACTGATTATATCATCTGAACCTTGAGCAGTGA |
| | | AAACCATGTTTTGAGGTAAATGTCTGATGATTGAGGAA |
| | | ATCAGGCCTGGTTGGGCATCAGCCAAGTCCTTTAAAAG |
| | | GAGACCATGTGAGTACTTGCTTTGCTCTTTGAAGGACTT |
| | | CTCATCGTGGGGAAATCTGTAACAATGTATGTAGTTGCC |
| | | CGTGTCAGGCTGGTAGATGGCCATTTCCACCGGATCATT |
| | | TGGTGTTCCTTCAATGTCAATCCATGTGGTAGCTTTTGA |
| | | ATCAAGCATCTGAATTGAGGACACAACAGTATCTTCTTT |
| | | CTCCTTAGGGATTTGTTTAAGGTCCGGTGATCCTCCGTTT |
| | | CTTACTGGTGGCTGGATAGCACTCGGCTTCGAATCTAAA |
| | | TCTACAGTGGTGTTATCCCAAGCCCTCCCTTGAACTTGA |
| | | GACCTTGAGCCAATGTAAGGCCAACCATCCCCTGAAAG |
| | | ACAAATCTTGTATAGTAAATTTTCATAAGGATTTCTCTG |
| | | TCCGGGTGTAGTGCTCACAAACATACCTTCACGATTCTT |
| | | TATTTGCAATAGACTCTTTATGAGAGTACTAAACATAGA |
| | | AGGCTTCACCTGGATGGTCTCAAGCATATTGCCACCATC |
| | | AATCATGCAAGCAGCTGCTTTGACTGCTGCAGACAAACT |
| | | GAGATTGTACCCTGAGATGTTTATGGCTGATGGCTCATT |
| | | ACTAATGATTTTTAGGGCACTGTGTTGCTGTGTGAGTTT |
| | | CTCTAGATCTGTCATGTTCGGGAACTTGACAGTGTAGAG |
| | | CAAACCAAGTGCACTCAGCGCTTGGACAACATCATTAA |
| | | GTTGTTCACCCCCTTGCTCAGTCATACAAGCGATGGTTA |
| | | AGGCTGGCATTGATCCAAATTGATTGATCAACAATGTAT |
| | | TATCCTTGATGTCCCAGATCTTCACAACCCCATCTCTGTT |
| | | GCCTGTGGGTCTAGCATTAGCGAACCCCATTGAGCGAA |
| | | GGATTTCGGCTCTTTGTTCCAACTGAGTGTTTGTGAGAT |
| | | TGCCCCCATAAACACCAGGCTGAGACAAACTCTCAGTTC |
| | | TAGTGACTTTCTTTCTTAACTTGTCCAAATCAGATGCAA |
| | | GCTCCATTAGCTCCTCTTTGGCTAAGCCTCCCACCTTAA |
| | | GCACATTGTCCCTCTGGATTGATCTCATATTCATCAGAG |
| | | CATCAACCTCTTTGTTCATGTCTCTTAACTTGGTCAGATC |
| | | AGAATCAGTCCTTTTATCTTTGCGCATCATTCTTTGAACT |
| | | TGAGCAACTTTGTGAAAGTCAAGAGCAGATAACAGTGC |
| | | TCTTGTGTCCGACAACACATCAGCCTTCACAGGATGGGT |
| | | CCAGTTGGATAGACCCCTCCTAAGGGACTGTACCCAGC |
| | | GGAATGATGGGATGTTGTCAGACATTTTGGGGTTGTTTG |
| | | CACTTCCTCCGAGTCAGTGAAGAAGTGAACGTACAGCG |
| | | TGATCTAGAATCGCCTAGGATCCACTGTGCG |
| 7 | Nucleotide | GCGCACCGGGGATCCTAGGCATACCTTGGACGCGCATA |
| | sequence of PICV | TTACTTGATCAAAGATGCATGGTGACACCCCCACCCTGC |
| | HPV16 E7E6- | ATGAGTACATGCTGGACCTGCAGCCAGAGACCACAGAC |
| | GP-S-segment 2 | CTGTATGGCTATGGCCAGCTGAATGACAGCAGTGAGGA |
| | (The genomic | AGAGGATGAGATTGATGGGCCAGCAGGCCAGGCAGAAC |
| | segment is RNA, | CTGACAGAGCCCACTACAACATTGTCACCTTCTGCTGCA |
| | the sequence in | AGTGTGACAGCACCCTGAGACTGTGTGTGCAGAGCACC |
| | SEQ ID NO: 7 is | CATGTGGACATCAGAACCCTGGAAGACCTGCTGATGGG |
| | shown for DNA; | CACCCTGGGCATTGTGGGCCCCATCTGCTCCCAGAAGCC |
| | however, | CCACCAGAAAAGAACTGCCATGTTCCAGGACCCCCAGG |
| | exchanging all | AGAGGCCCAGAAAGCTGCCCCAGCTCTGCACAGAGCTG |
| | thymidines (“T”) | CAGACCACCATCCATGACATCATCCTGGAATGTGTCTAC |
| | in SEQ ID NO: 7 | TGCAAGCAGCAGCTGCTGAGGAGAGAGGTGTATGACTT |
| | for uridines (“U”) | TGCCTTCAGGGACCTGTGCATTGTGTACAGGGATGGCAA |
| | provides the RNA | CCCCTATGCTGTGGGGGACAAGTGCCTCAAGTTCTACAG |
| | sequence.) | CAAGATCAGTGAGTACAGGCACTACTGCTACAGCCTGT |
| | | ATGGCACCACCCTGGAACAGCAGTACAACAAGCCCCTG |
| | | TGTGACCTCCTGATCAGATGCATCAATGGCCAGAAACCC |
| | | CTCTGCCCTGAGGAAAAGCAGAGACACCTGGACAAGAA |
| | | GCAGAGGTTCCACAACATCAGAGGCAGGTGGACAGGCA |
| | | GATGCATGAGCTGCTGCAGAAGCAGCAGAACCAGAAGA |
| | | GAGACCCAGCTGTGAGCCCTAGCCTCGACATGGGCCTC |
| | | GACGTCACTCCCCAATAGGGGAGTGACGTCGAGGCCTC |
| | | TGAGGACTTGAGCTTATTTACCCAGTCTCACCCATTTGT |
| | | AGGGTTTCTTTGGGATTTTATAATACCCACAGCTGCAAA |
| | | GAGAGTTCCTAGTAATCCTATGTGGCTTCGGACAGCCAT |
| | | CACCAATGATGTGCCTATGAGTGGGTATTCCAACTAAGT |
| | | GGAGAAACACTGTGATGGTGTAAAACACCAAAGACCAG |
| | | AAGCAAATGTCTGTCAATGCTAGTGGAGTCTTACCTTGT |
| | | CTTTCTTCATATTCTTTTATCAGCATTTCATTGTACAGAT |
| | | TCTGGCTCTCCCACAACCAATCATTCTTAAAATGCGTTT |
| | | CATTGAGGTACGAGCCATTGTGAACTAACCAACACTGC |
| | | GGTAAAGAATGTCTCCCTGTGATGGTATCATTGATGTAC |
| | | CAAAATTTTGTATAGTTGCAATAAGGGATTTTGGCAAGC |
| | | TGTTTGAGACTGTTTCTAATCACAAGTGAGTCAGAAATA |
| | | AGTCCGTTGATAGTCTTTTTAAAGAGATTCAACGAATTC |
| | | TCAACATTAAGTTGTAAGGTTTTGATAGCATTCTGATTG |
| | | AAATCAAATAACCTCATCGTATCGCAAAATTCTTCATTG |
| | | TGATCTTTGTTGCATTTTGCCATCACAGTGTTATCAAAA |
| | | CATTTTATTCCAGCCCAAACAATAGCCCATTGCTCCAAA |
| | | CAGTAACCACCTGGGACATGTTGCCCAGTAGAGTCACTC |
| | | AAGTCCCAAGTGAAAAAGCCAAGGAGTTTCCTGCTCAC |
| | | AGAACTATAAGCAGTTTTTTGGAGAGCCATCCTTATTGT |
| | | TGCCATTGGAGTATATGTACAGTGATTTTCCCATGTGGT |
| | | GTTCTGTATGATCAGGAAATTGTAATGTGTCCCACCTTC |
| | | ACAGTTTGTTAGTCTGCAAGACCCTCCACTACAGTTATT |
| | | GAAACATTTTCCAACCCACGCAATTTTTGGGTCCCCAAT |
| | | GATTTGAGCAAGCGACGCAATAAGATGTCTGCCAACCT |
| | | CACCTCCTCTATCCCCAACTGTCAAGTTGTACTGGATCA |
| | | ACACCCCAGCACCCTCAACTGTTTTGCATCTGGCACCTA |
| | | CATGACGAGTGACATGGAGCACATTGAAGTGTAACTCA |
| | | TTAAGCAACCATTTTAATGTGTGACCTGCTTCTTCTGTCT |
| | | TATCACAATTACTAATGTTACCATATGCAAGGCTTCTGA |
| | | TGTTGGAAAAGTTTCCAGTAGTTTCATTTGCAATGGATG |
| | | TGTTTGTCAAAGTGAGTTCAATTCCCCATGTTGTGTTAG |
| | | ATGGTCCTTTGTAGTAATGATGTGTGTTGTTCTTGCTACA |
| | | TGATTGTGGCAAGTTGTCAAACATTCTTGTGAGGTTGAA |
| | | CTCAACGTGGGTGAGATTGTGCCTCCTATCAATCATCAT |
| | | GCCATCACAACTTCTGCCAGCCAAAATGAGGAAGGTGA |
| | | TGAGTTGGAATAGGCCACATCTCATCAGATTGACAAATC |
| | | CTTTGATGATGCATAGGGTTGAGACAATGATTAAGGCG |
| | | ACATTGAACACCTCCTGCAGGACTTCGGGTATAGACTGG |
| | | ATCAAAGTCACAACTTGTCCCATTTTGGGGTTGTTTGCA |
| | | CTTCCTCCGAGTCAGTGAAGAAGTGAACGTACAGCGTG |
| | | ATCTAGAATCGCCTAGGATCCACTGTGCG |
| 8 | Nucleotide | GCGCACCGGGGATCCTAGGCATCTTTGGGTCACGCTTCA |
| | sequence of PICV | AATTTGTCCAATTTGAACCCAGCTCAAGTCCTGGTCAAA |
| | L-segment | ACTTGGGATGGGACTCAGATATAGCAAAGAGGTCAGGA |
| | (The genomic | AGAGACATGGCGACGAAGATGTGGTGGGAAGGGTCCCC |
| | segment is RNA, | ATGACCCTCAATCTACCACAGGGCCTGTATGGCAGGTTC |
| | the sequence in | AACTGCAAATCTTGCTGGTTCGTCAACAAAGGTCTCATC |
| | SEQ ID NO: 8 is | AGGTGCAAAGACCACTATCTGTGTCTTGGGTGCTTAACC |
| | shown for DNA; | AAAATGCACTCCAGAGGCAATCTCTGCGAGATATGCGG |
| | however, | CCACTCACTGCCAACCAAGATGGAGTTCCTAGAAAGCC |
| | exchanging all | CCTCTGCACCACCCTACGAGCCATAAACCAGGGCCCCTG |
| | thymidines (“T”) | GGCGCACCCCCCTCCGGGGGTGCGCCCGGGGGCCCCCG |
| | in SEQ ID NO: 8 | GCCCCATGGGGCCGGTTGTTTACTCGATCTCCACTGACT |
| | for uridines (“U”) | CATTGTCCTCAAACAACTTTCGACACCTGATTCCCTTGA |
| | provides the RNA | TCTTGAAGGGTCCTGTCTCGTCTGCAATCATAACAGATC |
| | sequence.) | CTAGAGTCTTACTTCTTATTATACTAAAGTGACCACAAT |
| | | TCAACCAATCTTTGGCATCATGCAACATGTGTTCAAACA |
| | | CTTCGGGGAAATTTTCAATCATGAGTCTTAAATCCTGCT |
| | | CGTTCATACTTATTCCCTTGTTGTGAGACTGTGCACTTGA |
| | | AAGGTACTGAAAAAGGTTGGCAATAAATCTTGGCCTTTT |
| | | CTCAGGTTCTAATGCTTCCAGTGCAATGATGACCACCTT |
| | | TGAGTCTAAGTTCACTTCCAATCTAGAAACCACTCTGTT |
| | | GCCCTCTTTGATCAACCCACCCTCTAAAATGAGGGGTTG |
| | | CATCCCAACATCAGGACCAATCAACTTATAGGAAAATTT |
| | | GTTTTTCAAATCCTTGAAACGATTTTTCAAATCTATTCTC |
| | | ACCTTCTGGAACACAGTTGACCTTGACTTGAAGTGAATG |
| | | TCTTGACCTTCCAATAGATCATTGAAGTCTAGAACATCT |
| | | TTTCCGTTGATGAGAGGATTCAGAACCAAAAGTGACAC |
| | | ACCATCCAGACTTATGTGATTCCCGGAAGATTGAGAAA |
| | | CATAATACTCAACAGAATGGGGGTTCAACAATAGGTAA |
| | | CCATCAGAGTCCAATGAGTCCAGCAATGACTCCCTTTCA |
| | | ATAAGAAATCTTAATTTTAATATGTAATTGGTAGACCTC |
| | | TCATATCTAAATTTGTGGCTCACTCTCTTATGAGAAAAT |
| | | GTTAGGTTGAGCTCAATGGGAATGACCTCAGAAGGTGA |
| | | TGCTAAAATGAGTTGTTCAATGTTCTCATAGTTATCTCT |
| | | ATTCACCCAGTCAAGTTCATTAATAAATACACTAATGTT |
| | | CAAATTAACACAGGACAAAATCAGTTTGCTGCTTACAA |
| | | AGCCAACATCCAAGTCATCCAGATTCATTGTCCTAGAAG |
| | | TGTTATTCTTTTTGCAGTCACAAATGAACTGGGTTAATT |
| | | GTTTCAGATCATGTTGTGCATTGTTTGGCAACAATTCAA |
| | | GCTCACCAAACCAAAAATATTTCTTGAACTGAGATGTTG |
| | | ACATAATCACAGGCACCAACATTGACTCAAACAAAATC |
| | | TGTATCAAGAAATTTGTGCACACTTCTTCTGGTTCAAGG |
| | | TTGAATCCTCTCTCCAGTGGATGAGACTCTCTGCTATGG |
| | | GACATTGCAAGCTCATTTTGCTTTACAATATACAATTCT |
| | | TCTCTGCGATGTTTTATAATATGACTAACAATACCAAGA |
| | | CATTCTGATGTTATATCAATTGCCACACAAAGGTCTAAG |
| | | AACTTTATCCTCTGAACCCATGATAGCCTCAGCATATTC |
| | | AAATCAGACAGGAAAGGGGATATGTGTTCATCAAATAG |
| | | TGTAGGGAAGTTCCTCCTGATTGAGTAAAGTATGTGGTT |
| | | GATGCCCACCTTGTCCTCAAGCTCAGAATGTGTGCTTGG |
| | | TTTTATTGGCCAGAAGTGATTGGGATTGTTTAGGTGAGT |
| | | GACTATCTTGGGTACTTCAGCTTTTTGAAACACCCAGTT |
| | | ACCCAACTCGCAAGCATTGGTTAACACAAGAGCAAAAT |
| | | AATCCCAAATTAAGGGTCTGGAGTACTCACTTACTTCAC |
| | | CAAGTGCTGCTTTACAATAAACACCTTTGCGCTGATTAC |
| | | AAAAGTGACAATCACGGTGTAAGATAATCTTGCTTGTA |
| | | ATATCCCTGATATACTTAAATCCTCCTTTCCCATCTCTTA |
| | | CACATTTTGAGCCCATACTTTTGCAAACTCCTATGAATC |
| | | CTGATGCTATGCTGCTCTGAAAAGCTGATTTGTTGATAG |
| | | CATCAGCCAAAATCTTCTTAGCCCCTCTGACATAGTTCT |
| | | TTGATAATTTGGACTGTACGGATTTGACAAGACTGGGTA |
| | | TTTCTTCTCGCTGCACAGTTCTTGTTGTGCTCATTAACTT |
| | | AGTACGAAGCACCAATCTGAGATCACCATGAACCCTTA |
| | | AATTTAACCACCTAATATTAAGAGCATCCTCAATAGCCT |
| | | CAGTCTCGACATCACAAGTCTCTAATAACTGTTTTAAGC |
| | | AGTCATCCGGTGATTGCTGAAGAGTIGTTACAATATAAC |
| | | TTTCTTCCAGGGCTCCAGACTGTATTTTGTAAAATATTTT |
| | | CCTGCATGCCTTTCTGATTATTGAAAGTAGCAGATCATC |
| | | AGGAAATAGTGTCTCAATTGATCGCTGAAGTCTGTACCC |
| | | TCTCGACCCATTAACCCAATCGAGTACATCCATTTCTTC |
| | | CAGGCACAAAAATGGATCATTTGGAAACCCACTATAGA |
| | | TTATCATGCTATTTGTTCGTTTTGCAATGGCCCCTACAAC |
| | | CTCTATTGACACCCCGTTAGCAACACATTGGTCCAGTAT |
| | | TGTGTCAATTGTATCTGCTTGCTGATTGGGTGCTTTAGCC |
| | | TTTATGTTGTGTAGAGCTGCAGCAACAAACTTTGTAAGG |
| | | AGGGGGACTTCTTGTGACCAAATGAAGAATCTCGATTTG |
| | | AACTCACTTGCAAAGGTCCCCACAACTGTTTTAGGGCTC |
| | | ACAAACTTGTTGAGTTTGTCTGATAGAAAGTAGTGAAAC |
| | | TCCATACAGTCCAATACCAATTCAACATTCAACTCATCT |
| | | CTGTCCTTAAATTTGAAACCCTCATTCAAGGATAACATG |
| | | ATCTCATCATCACTCGAAGTATATGAGATGAACCGTGCT |
| | | CCATAACAAAGCTCCAATGCGTAATTGATGAACTGCTCA |
| | | GTGATTAGACCATATAAGTCAGAGGTGTTGTGTAGGAT |
| | | GCCCTGACCCATATCTAAGACTGAAGAGATGTGTGATG |
| | | GTACCTTGCCCTTCTCAAAGTACCCAAACATAAATTCCT |
| | | CTGCAATTGTGCACCCCCCTTTATCCATCATACCCAACC |
| | | CCCTTTTCAAGAAACCTTTCATGTATGCCTCAACGACAT |
| | | TGAAGGGCACTTCCACCATCTTGTGAATGTGCCATAGCA |
| | | ATATGTTGATGACTGCAGCATTGGGAACTTCTGACCCAT |
| | | CTTTGAGTTTGAACTCAAGACCTTTTAATAATGCGGCAA |
| | | AGATAACCGGCGACATGTGTGGCCCCCATTTTGAATGGT |
| | | CCATTGACACCGCAAGACCACTTTGCCTAACAACTGACT |
| | | TCATGTCTAATAATGCTCTCTCAAACTCTTTCTCGTTGTT |
| | | CAGACAAGTATACCTCATGTTTTGCATAAGGGATTCAGA |
| | | GTAATCCTCAATGAGTCTGGTTGTGAGTTTAGTATTTAA |
| | | ATCACCGACATAAAGCTCCCTGTTGCCACCCACCTGTTC |
| | | TTTATAAGAAAGACCAAATTTCAATCTCCCTACATTGGT |
| | | GGATACACCAGACCTCTCTGTGGGAGACTCATCTGAATA |
| | | GAAACAGAGATTTCGTAAGGATGAGTTGGTAAAAAAGC |
| | | TTTGATCCAATCTTTTAGCTATCGATTCAGAATTGCTCTC |
| | | TCTTGAGCTTATACGTGATGTCTCTCTAATTTGTAGTGCT |
| | | GCATCTGTGAACCCAAGTCTGCTTCTACTTTTGTGATCA |
| | | TATCTTCCGACTCGATTATCATAATCGCTTGCAATGAGA |
| | | ATGTATTTAAAGCACTCAAAATAATCAGCTTCTTTGTAC |
| | | GCCTTCAATGTGAGGTTCTTTATTAAAAACTCCAGAGGA |
| | | CACGGATTCATTAGTCTGTCTGCAAAGTACACTGATCTA |
| | | GCAGTGACATCCTCATAGATCAAGTTTACAAGATCCTCA |
| | | TACACTTCTGCTGAAAACAGGCTGTAATCAAAATCCTTT |
| | | ACATCATGAAGTGAAGTCTCTCTTTTGATGACAACCATT |
| | | GTCGATTTGGGCCATAATCTCTCTAGTGGACATGAAGTC |
| | | TTAAGGTTGGTTTTGACATTGGTGTCAACCTTAGACAAT |
| | | ACTTTTGCAACTCTGGTCTCAATTTCTTTAAGACAGTCA |
| | | CCCTGATCTTCTGATAGTAACTCTTCAACTCCATCAGGC |
| | | TCTATTGACTCCTTTTTTATTTGGATCAATGATGACAACC |
| | | TCTTCAGAATCTTGAAATTTACCTCCTTTGGATCTAACTT |
| | | GTATTTACCCTTAGTTTTGAAATGTTCAATCATTTCCACA |
| | | ACAACAGCAGACACAATGGAAGAGTAATCATATTCAGT |
| | | GATGACCTCACCAACTTCATTGAGTTTTGGAACCACCAC |
| | | ACTTTTGTTGCTGGACATATCCAAGGCTGTACTTGTGAA |
| | | GGAGGGAGTCATAGGGTCACAAGGAAGCAGGGGTTTCA |
| | | CTTCCAATGAGCTACTGTTAAATAGTGATAGACAAACAC |
| | | TAAGTACATCCTTATTCAACCCCGGCCTTCCCTCACATTT |
| | | GGATTCCAGCTTTTTACCAAGTAGTCTCTCTATATCATG |
| | | CACCATCTTCTCTTCTTCCTCAGTAGGAAGTTCCATACTA |
| | | TTAGAAGGGTTGACCAAGACTGAATCAAACTTTAACTTT |
| | | GGTTCCAAGAACTTCTCAAAACATTTGATTTGATCAGTT |
| | | AATCTATCAGGGGTTTCTTTGGTTATAAAATGGCATAAA |
| | | TAGGAGACATTCAAAACAAACTTAAAGATCTTAGCCAT |
| | | ATCTTCCTCTCTGGAGTTGCTGAGTACCAGAAGTATCAA |
| | | ATCATCAATAAGCATTGCTGTCTGCCATTCTGAAGGTGT |
| | | TAGCATAACGACTTTCAATTTCTCAAACAATTCTTTAAA |
| | | ATGAACTTCATTTACAAAGGCCATAATGTAATATCTAAA |
| | | GCCTTGCAAGTAAACTTGAATACGCTTGGAAGGGGTGC |
| | | ACAGTATGCAGAGAATAAGTCGTCTGAGTAAATCAGAA |
| | | ACAGAATCCAAGAGGGGTTGGGACATAAAGTCCAACCA |
| | | GGATAACATCTCCACACAAGTCCTTTGAATCACATCTGC |
| | | ACTAAAGATCGGTAAGAAAAATCTCTTGGGATCACAGT |
| | | AAAAAGACGCTTTTGTTTCATACAAACCCCCACTTTTGG |
| | | ATCTATAAGCAACAGCATAACACCTGGACCTCTCCCCTG |
| | | TCTTCTGGTACAGTAGTGTGAGAGAACCTCCTTCTCCAA |
| | | ATCGCTGGAAGAAAACTTCGTCACAGTAAACCTTCCCAT |
| | | AAAACTCATCAGCATTGTTCACCTTCATCTTAGGAACTG |
| | | CTGCTGTCTTCATGCTATTAATGAGTGACAAACTCAAAC |
| | | TTGACAATGTTTTCAGCAATTCCTCAAACTCACTTTCGC |
| | | CCATGATGGTATAATCAGGCTGCCCTCTTCCTGGCCTAC |
| | | CCCCACACATACACTGTGACTTTGTCTTGTATTGAAGAC |
| | | AGGGTTTAGCACCCCATTCATCTAACACTGATGTTTTCA |
| | | GATTGAAGTAATATTCAACATCAGGTTCCCGTAGAAGA |
| | | GGGAGAATGTCATCAAGGGGAAGTTCACCACAGACCGA |
| | | GCTCAGTCTCTTCTTAGCCTTCTCTAACCAGTTGGGGTTT |
| | | TTAATGAATTTTTTAGTGATTTGTTCCATCAGGAAGTCG |
| | | ACATTAATCAACCTGTCATTTACAGACGGTAACCCTTGC |
| | | ATTAGGAGCACCTCTCTGAACACAGCACCTGGAGAAGA |
| | | CTTGTCCAAGTCACACAAAATGTTGTACATGATAAGGTC |
| | | CAGAACCAACATGGTGTTCCTCCTTGTGTTAAAAACCTT |
| | | TTGAGACTTAATTTTGTTGCATATTGAAAGTACTCTAAA |
| | | ATATTCTCTGCTTTCAGTTGATGAATGCTTGACCTCAGA |
| | | TTGCCTGAGTTGGCCTATTATGCCCAAAATGTGTACTGA |
| | | GCAAAACTCACATAATCTGATTTCTGATTTAGGTACATC |
| | | TTTGACAGAACATTGGATAAATTCATGGTTCTGAAGTCT |
| | | AGAAATCATATCTTCCCTATCTGTAGCCTGCAGTTTCCT |
| | | ATCGAGTTGACCAGCAAGTTGCAACATTTTAAATTGCTG |
| | | AAAGATTTCCATGATTTTTGTTCTACATTGATCTGTTGTC |
| | | AGTTTATTATTAATGCCAGACATTAATGCCTTTTCCAAC |
| | | CTCACTTTGTAAGGAAGTCCCCTTTCCTTTACAGCAAGT |
| | | AGTGACTCCAGACCGAGACTCTGATTTTCTAAGGATGAG |
| | | AGGGAACTTATAAGGCGTTCGTACTCCAACTCCTCAACT |
| | | TCTTCACCAGATGTCCTTAATCCATCCATGAGTTTTAAA |
| | | AGCAACCACCGAAGTCTCTCTACCACCCAATCAGGAAC |
| | | AAATTCTACATAATAACTGGATCTACCGTCAATAACAGG |
| | | TACTAAGGTTATGTTCTGTCTCTTGAGATCAGAACTAAG |
| | | CTGCAACAGCTTCAAAAAGTCCTGGTTGTATTTCTTCTC |
| | | AAATGCTTCTTGACTGGTCCTCACAAACACTTCCAAAAG |
| | | AATGAGGACATCTCCAACCATACAGTAACCATCTGGTGT |
| | | AACATCCGGCAATGTAGGACATGTTACTCTCAACTCCCT |
| | | AAGGATAGCATTGACAGTCATCTTTGTGTTGTGTTTGCA |
| | | GGAGTGTTTCTTGCATGAATCCACTTCCACTAGCATGGA |
| | | CAAAAGCTTCAGGCCCTCTATCGTGATGGCCCTATCTTT |
| | | GACTTGTGCAAGAACGTTGTTTTTCTGTTCAGATAGCTC |
| | | TTCCCATTCGGGAACCCATTTTCTGACTATGTCTTTAAGT |
| | | TCGAAAACGTATTCCTCCATGATCAAGAAATGCCTAGG |
| | | ATCCTCGGTGCG |

The nucleotide sequence of SEQ ID NOs: 1 and 2 encode the HPV16 E7E6 fusion protein. The nucleotide sequence of the HPV16 E7E6 fusion protein of SEQ ID NOs: 1 and 2 are modified to reduce CpG dinucleotide motifs (dinucleotide optimized). Arenavirus particles comprising SEQ ID No: 1 or 2 can demonstrate improved genetic stability, improved expression and improved immunogenicity in the methods provided herein. Assays to demonstrate these properties are described below. The attenuated, replication-competent viral vector can be generated de novo using a cDNA rescue system comprising plasmids encoding the two short (S) genome segments including the dinucleotide optimized E7E6 nucleotide sequence as well as the gene for the arenaviral nucleoprotein (NP) or arenaviral glycoprotein (GP), respectively, and the long (L) genome segment including the genes for the RING finger protein Z and the RNA-directed RNA polymerase L.

### Assays to Demonstrate Clinical Benefit of Dinucleotide Optimized Sequences

The following assays may be used to demonstrate the improvement of the dinucleotide optimized sequences of SEQ ID NOs: 1 or 2.

Genetic Stability

The tri-segmented arenavirus particle encoding the dinucleotide optimized HPV16 E7E6 nucleotide sequence can have a stable expression of the encoded HPV antigen after being passaged multiple generations, which is necessary for larger-scale commercial production. In some embodiments, the tri-segmented arenavirus particle can have stable expression of the HPV antigen after being passaged at least 4, 5, 6, 7, 8, 9, or 10 generations.

Serial passaging of vector candidates in the propagation cell line. Small-scale HEK293 cell cultures can be infected with replication competent vectors at MOI 0.001 (RCV FFU/ml titer). At day 4 post infection, supernatant can be cleared from cells and debris by centrifugation. Thereof determined RCV FFU titers can be used to generate the next passage by infecting fresh cells as described above. Vector stock material can be passaged for 9 sequential passages (up to passage p10).

Analysis of passaged vector material. Vector material generated de novo (P1) as well as derived from serial passages thereof can be subsequently analyzed for infectivity by FFU and RCV FFU assays. Transgene stability of the vector can be analyzed by isolating genomic vRNA from the virus containing supernatant of different passage levels, transcription into cDNA and subsequent amplification by PCR with transgene flanking primers specific for the respective transgene and S-Segment. Transgene expression of vector stocks can be confirmed by Western Blot analysis of cell lysates at different passage levels using transgene-specific antibodies.

Antigen Expression

The tri-segmented arenavirus particle encoding the dinucleotide optimized HPV16 E7E6 nucleotide sequence can have consistent expression of the encoded HPV fusion protein.

Analysis of expression level of the encoded E7E6 antigen. Western blot, ELISA, radioimmunoassay, immunoprecipitation, immunocytochemistry, or immunocytochemistry in conjunction with FACS can be used to quantify the gene products of the arenavirus S segment or tri-segmented arenavirus particle.

Western Blotting. Infected cells grown in tissue culture flasks or in suspension can be lysed at indicated time points post infection using RIPA buffer (Thermo Scientific) or used directly without cell-lysis. Samples can be heated to 99° C. for 10 minutes with reducing agent and NuPAGE LDS Sample buffer (NOVEX) and chilled to room temperature before loading onto NuPAGE 4-12% Bis-Tris SDS-gels for electrophoresis. Proteins can be blotted onto membranes using the Invitrogen iBlot Gel transfer Device. Finally, the membranes can be probed with primary antibodies directed against proteins of interest and horseradish peroxidase (HRP) conjugated secondary antibodies followed by staining with Immobilon Western Chemiluminescent HRP Substrate (Merck/Millipore)

Immunogenicity

The tri-segmented arenavirus particle encoding the dinucleotide optimized HPV16 E7E6 nucleotide sequence can induce strong immune responses against the encoded HPV fusion protein.

MHC-Peptide Multimer Staining Assay for Detection of Antigen-Specific CD8⁺ T-cells. Any assay well known in the art can be used to measure antigen-specific CD8⁺ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay can comprise a tetramer assay used to detect the presence of antigen specific T-cells. In order to detect an antigen-specific T-cell, it must bind to both, the peptide and the tetramer of MHC molecules custom made for a defined antigen specificity and MHC haplotype of T-cells (typically fluorescently labeled). T-cells that recognize the tetramer, and are thus specific for the antigen can then be detected by flow cytometry via the fluorescent label.

ELISPOT Assay for Detection of Antigen-Specific T-cells. Any assay well-known in the art can be used to test antigen-specific T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8) as exemplified in Table 1. Summary of Sample Collection for Central Laboratory Analyses, cytokines such as but not limited to IFN-γ can be measured by the ELISPOT assay. Briefly, the assay comprises the following steps: An immunospot plate is coated with an anti-cytokine antibody. Cells are then incubated in the immunospot plate with peptides derived from the antigen of interest. Antigen-specific cells secrete cytokines, which bind to the coated antibodies. The cells are then washed off and a second biotyinlated-anticytokine antibody is added to the plate and visualized with an avidin-HRP system or other appropriate methods.

TABLE 1
Summary of Sample Collection for Central Laboratory Analyses

| Category | Sample Type | Type of Analyses |
|---|---|---|
| Viral Shedding | Saliva, feces, blood, | Viral shedding is analyzed by |
| | and urine | quantitative reverse transcription PCR to |
| | | quantify the copies of nucleoprotein RNA |
| Viral Infectivity | Serum, urine, and | Viral Infectivity is analyzed by RCV |
| (RCV) | saliva | assay to measure the number of replication |
| | | competent viral vectors |
| Biomarker | Blood/plasma | Exome and mutational changes (ctDNA) |
| | | Transcriptional analysis (RNA-seq) |
| Biomarker | Serum | Cytokines |
| | | Neutralizing antibodies |
| | | bAb (anti-PAP, anti-PSA, anti-PSMA |
| | | antibodies) |
| Biomarker | Tumor tissue | IHC TIL |
| | | Transcriptome analysis (RNA-seq) |
| | | WES analysis |
| Immunogenicity | Blood | Intracellular cytokine staining (ICS) |
| | | panel CD4 and CD8: IFN-γ, TNF-α, IL-2, |
| | | CD107a, and CD154 from PBMC samples |
| Immunogenicity | Blood | ELISpot assay measuring secreted IFN-γ |
| | | using PSA, PAP, and PSMA-based peptides and |
| | | LCMV NP peptides from PBMC samples |

Abbreviations: bAb = binding antibody assay, CD4 = cluster of differentiation 4, CD8 = cluster of differentiation 8, ctDNA = circulating tumor deoxyribonucleic acid, ELISpot = enzyme-linked immune absorbent spot, ICS = intracellular cytokine staining, IFN-γ = interferon-gamma, IHC = immunohistochemistry, LCMV = lymphocytic choriomeningitis virus, NP = nucleoprotein, PAP = prostatic acid phosphatase, PBMC = peripheral blood mononuclear cell, PSA = prostate-specific antigen, PSMA = prostate-specific membrane antigen, RCV = replication-competent virus, RNA = ribonucleic acid, TIL = tumor-infiltrating lymphocyte, TNF α = tumor necrosis factor alpha, WES = whole exome sequencing.

Intracellular Cytokine Assay for Detection of Functionality of CD8⁺ and CD4⁺ T-cells. Any assay well-known in the art can be used to test the functionality of CD8⁺ and CD4⁺ T cell responses. For example, the intracellular cytokine assay combined with flow cytometry can be used as exemplified but not limited to Table 1. Summary of Sample Collection for Central Laboratory Analyses (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: upon activation of cells via specific peptides or protein, an inhibition of protein transport (e.g., brefeldin A) is added to retain the cytokines within the cell. After a defined period of incubation, typically 5 hours, a washing step follows, and antibodies to other cellular markers can be added to the cells. Cells are then fixed and permeabilized. The flurochrome-conjugated anti-cytokine antibodies are added and the cells can be analyzed by flow cytometry.

Serum ELISA. Determination of the humoral immune response upon vaccination of animals (e.g., mice, guinea pigs) can be done by antigen-specific serum ELISA's (enzyme-linked immunosorbent assays). In brief, plates can be coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

It is understood that modifications which do not substantially affect the activity of the various embodiments of this disclosure are also provided within the definition of the disclosure provided herein. Accordingly, the following examples are intended to illustrate but not limit the present disclosure.

### Example I

Arenavirus-Based Cancer Immunotherapy, Alone or in Combination with an Immune Checkpoint Inhibitor, in Patients with HPV16⁺ Cancers

This example describes an immunotherapy treatment strategy using arenavirus based particles alone or in combination with an immune checkpoint inhibitor.

The arenavirus particle that can be used in this treatment strategy includes Construct 1 and Construct 2, HPV antigen constructs described in FIG. 2B. Moreover, the antigen constructs for the HPV16 E7/E6 antigen that can be used in the treatment strategy described herein include the antigens described in US Patent Application Publication US-2018-0179257-A1, published Jun. 28, 2018, which are incorporated herein by reference. In particular, in certain embodiments, the HPV16 E7/E6 antigen encoded by the arenavirus particles used in the described treatment strategy includes the amino acid sequence of SEQ ID NO: 10 of US Patent Application Publication US-2018-0179257-A1 (Construct 1 and Construct 2).

The immune checkpoint inhibitor used in this treatment strategy includes an anti-PD-1 immune checkpoint inhibitor. Immune checkpoint inhibitors that can be used in the treatment strategy described herein, including the anti-PD-1 immune checkpoint inhibitor, include those described in US Patent Application Publication US-2018-0344830-A1, published Dec. 6, 2018, which are incorporated herein by reference.

The intratumoral administration of the arenavirus particle used in this treatment strategy includes the methods described in US Patent Application Publication US-2020-0113995-A1, published Apr. 16, 2020, which are incorporated herein by reference.

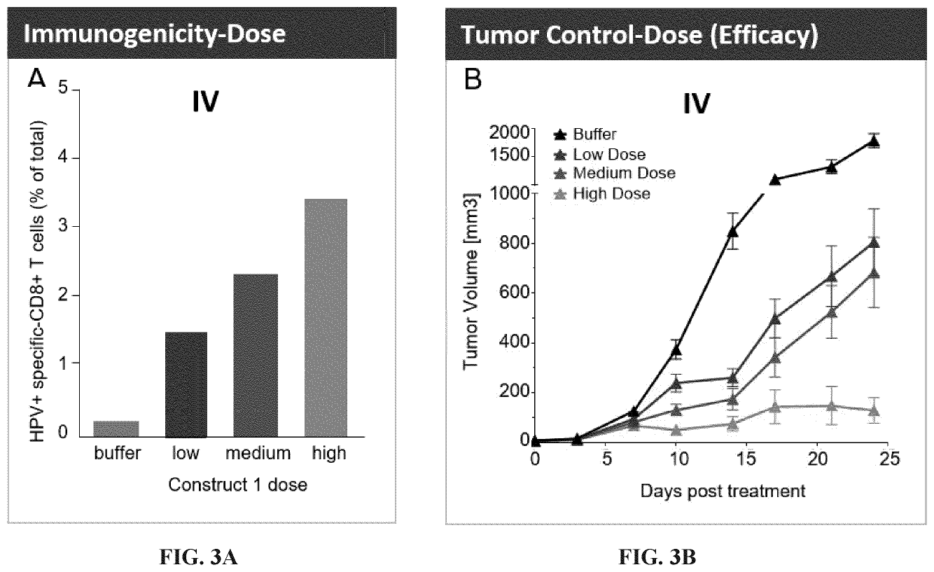

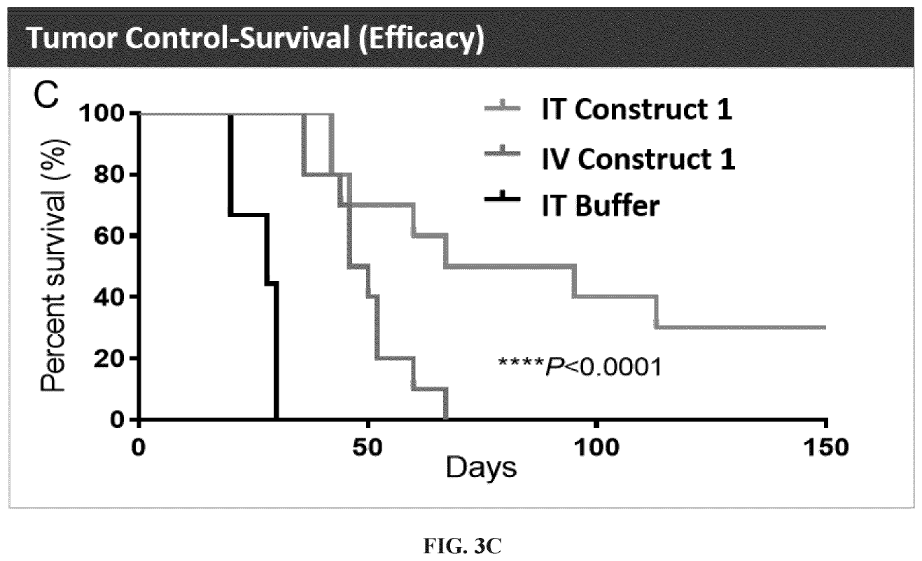

Construct 1 is a replication-competent live-attenuated vector based on the arenavirus LCMV encoding a non-oncogenic E7 and E6 fusion protein. In preclinical models, both intravenously (IV) and intratumorally (IT) administered Construct 1 demonstrates potent immunogenicity by induction of HPV16-specific cytotoxic T cells and associated efficacy (FIGS. 3A to 3C).

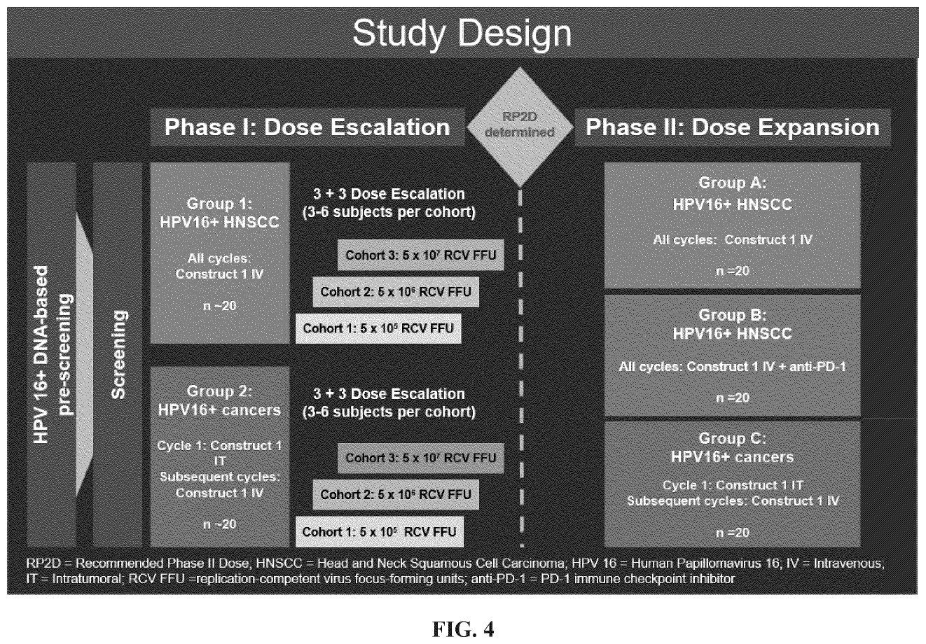

The treatment strategy for using an arenavirus-based cancer immunotherapy, alone or in combination with an immune checkpoint inhibitor, in patients with HPV16⁺ cancers is described in FIG. 4. This treatment strategy is a first in humans, Phase I/II study of Construct 1 monotherapy or in combination with PD-1 immune checkpoint inhibitor (anti-PD-1) in HPV16⁺ confirmed recurrent/metastatic cancers. Phase I consists of 2 treatment groups, each conducted with a 3⁺3 dose escalation design. Group 1 enrolls patients with HPV16⁺ head and neck squamous cell carcinoma who will receive Construct 1 IV only. Group 2 enrolls HPV16⁺ cancer patients with a safely accessible tumor site who will receive Construct 1 IT for the first dose, followed by Construct 1 IV for subsequent doses (IT-IV). Construct 1 can be administered every 21 days. The Phase II component can be conducted with the recommended Phase II doses (RP2Ds) defined in Phase I and can consist of 3 groups: Group A (Construct 1 IV only), Group B (Construct 1 IV plus anti-PD-1), and Group C (Construct 1 IT-IV).

Key inclusion and exclusion criteria for the treatment strategy includes the following:
All Patients—
Age ≥18 years
ECOG performance status of 0 to 1
At least one measurable lesion per RECIST 1.1 criteria that will be assessed for tumor response
Tumor progression or recurrence on standard of care therapy (including at least one systemic therapy) or a contraindication to standard of care
No untreated and/or symptomatic metastatic central nervous system disease, unless treated and stable for at least 4 weeks

Arenavirus based particles alone with IV administration—
Histologically confirmed HPV 16⁺ (genotype) head and neck squamous cell carcinoma
Tumor tissue collected following progression from last treatment, unless otherwise agreed

Arenavirus based particles with IT or IV administration—
Histologically confirmed HPV 16⁺ (genotype) cancer of any origin
Safe and accessible tumor site, amenable for biopsy and IT administration, unless otherwise agreed
At least one additional measurable lesion per RECIST 1.1 criteria, apart from the tumor site amenable for biopsy and IT administration

By employing the treatment strategy described above, the following study objectives and endpoints can be evaluated:
Primary
Phase I only: recommended Phase II dose of each group
Phase II only: preliminary antitumor activities of all groups (ORR)
Secondary
Safety and tolerability of all groups
Preliminary antitumor activities of all groups (PFS, OS, duration of response, disease control rate)
Exploratory
Preliminary immunogenicity (E7 and E6 antigen-specific assays, CD4⁺ and CD8⁺ T cell measurements)
Biomarkers correlating with immune and/or antitumor response in blood, tissue, serum, and plasma

Statistical analysis that can be conducted includes each group of the Phase I Dose Escalation part following a traditional 3⁺3 design, with at least 3 DLT-evaluable patients per dose level. For this viral-based therapy, the highest dose may not necessarily be the most efficacious. Backfill of cohorts can, therefore, be used to better assess safety and potential efficacy across doses.

For safety analysis, the number of treatment-emergent adverse events (TEAEs) and incidence rates can be tabulated by CTCAE grade. The incidence of treatment emergent abnormal laboratory, vital signs, and ECG values can also be summarized using descriptive statistics.

For efficacy analyses, all efficacy endpoints can be determined according to RECIST v1.1 and iRECIST. For Phase I, efficacy endpoints can be presented and no formal statistical testing needs to be performed. For Phase II, ORR and disease control rate can be summarized using exact 2-sided 95% CIs according to the Clopper-Pearson method. Duration of response, PFS, and OS can be performed using Kaplan-Meier curves.

### Example II

Immunogenicity of Arenavirus-Based Cancer Immunotherapy in Patients with Advanced HPV16⁺ Cancers

This example describes the immunogenicity of immunotherapy using arenavirus based particles, which result in changes of cytokine and chemokine, and the induction of tumor-antigen-specific T cells, in patients with advanced HPV16⁺ cancers.

Attenuated, replicating arenavirus vectors carrying non-oncogenic HPV16-specific E7 and E6 fusion protein were expressed in the genomic background of LCMV or PICV (i.e., Construct 1 and Construct 2, respectively; see FIG. 2B).

In previous pre-clinical studies, administering both LCMV-based arenavirus alone and sequential administration of PICV-based arenavirus followed by LCMV-based one (i.e., alternating 2-vector therapy) were shown to be safe and efficacious. The alternating 2-vector therapy induced E7- and E6-specific CD8⁺ T cell responses that accounted for up to 50% of circulating T cells (see Schmidt S, et al. Oncoimmunology. 2020; 9(1):1809960; Bonilla W, et al. Cell Rep Med. 2021; 2(3):1-17.).

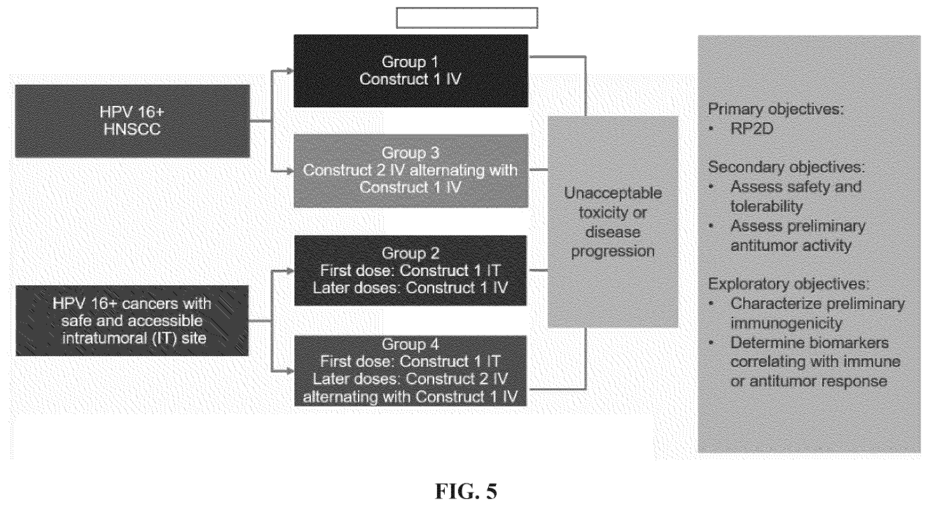

In the current example, immunogenicity results were obtained from the phase 1 portion of an open-label, first-in-human phase 1/2 clinical trial in heavily pretreated patients with prior failure of an anti-PD-1/PD-L1 and/or platinum-based chemotherapy for HPV16⁺ cancers. Construct 1 is a genetically engineered replication-competent tri-segmented arenavirus particle comprising two S-segments each encoding a fusion protein of HPV16 E7/E6, based on the LCMV strain Clone 13 with the viral surface glycoprotein from LCMV strain WE. Construct 2 is a genetically engineered replication-competent tri-segmented PICV particle comprising two S-segments each encoding a fusion protein of HPV16 E7/E6, based on virulent strain passage 18 of PICV. Different dose levels and schedules of monotherapy injections of Construct 1 alone or Construct 2 alternating with Construct 1 were analyzed. (see FIG. 5, Bonilla W, et al. Cell Rep Med. 2021; 2(3):1-17).

Patients had been enrolled in the phase 1/2 study. Cohort doses and the numbers of patients enrolled are as follows. For cohort 1 with Construct 1 monotherapy, the dosage was 5×10⁵ replication-competent virus focus-forming units (RCV FFU; n=13); for cohort 2 with Construct 1 monotherapy, the dosage was 5×10⁶ RCV FFU (n=13). For cohort 1 with Construct 1 and Construct 2 alternating monotherapy, the dosage for Construct 1 was 5×10⁶ RCV FFU, and the dosage for Construct 2 was 1×10⁶ RCV FFU (n=5); for cohort 2 with Construct 1 and Construct 2 alternating monotherapy, the dosage for Construct 1 was 5×10⁶ RCV FFU, and the dosage of Construct 2 was 1×10⁷ RCV FFU (n=1). 78.2% of the enrolled patients had head and neck squamous cell carcinoma, and 75% were males. The median age was 62 years old (the range of ages was 30 to 86 years old). 59.4% of the enrolled patients had an Eastern Cooperative Oncology Group (ECOG) performance status of 1, and the median prior lines of therapy was 3 (the range was 1 to 8). Measurement and analysis of the immunogenicity after the initial round of treatment are presented below (i.e., Example II). Specifically, the patients treated with Construct 1 monotherapy received 5×10⁶ RCV FFU of Construct 1. The patients who were treated with Construct 2 monotherapy, but not with Construct 1 yet by the time of the data collection, received 1×10⁶ RCV FFU of Construct 2.

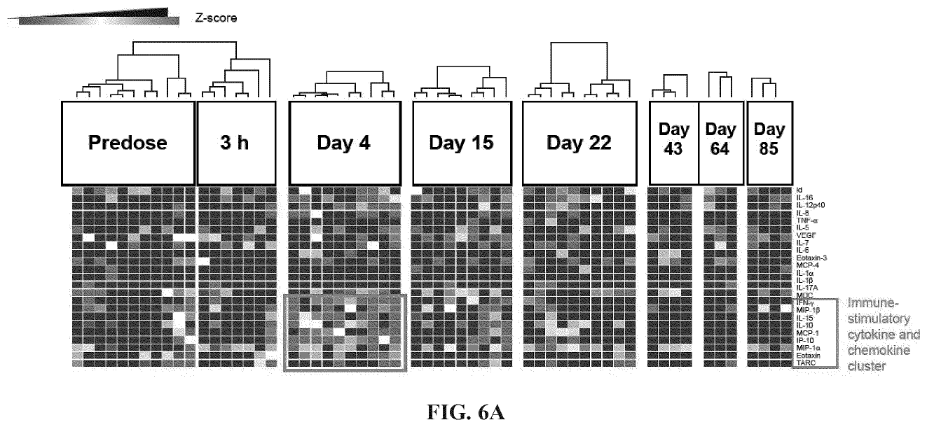

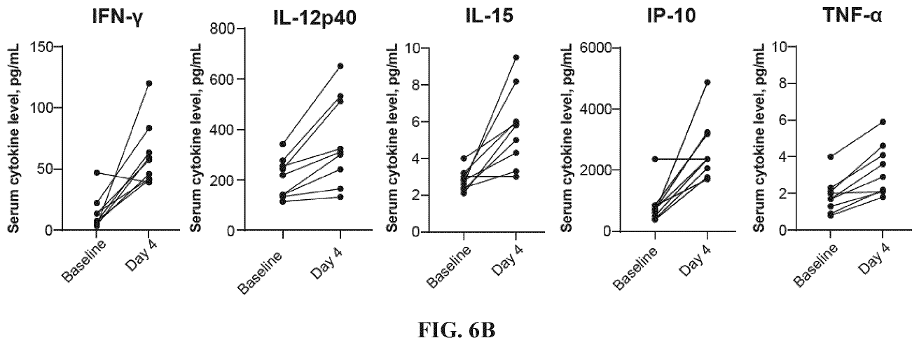

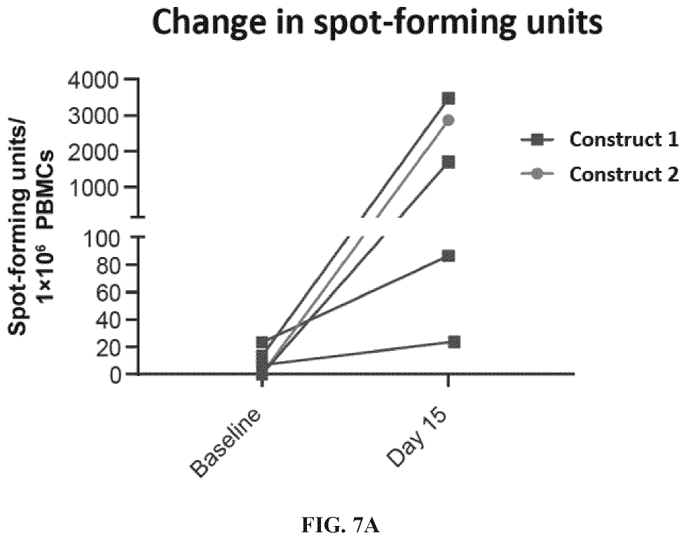

Serum cytokine and chemokine patterns from 66 samples (12 patients at up to 13 time points) were evaluated by the 30-plex Meso Scale Discovery (MSD) panel. An IFN-γ signature in serum post-treatment is an early sign of immune activation. Hierarchical clustering of serum 30-plex analysis showed that IFN-γ levels increased in 90% of patients after a single administration of Construct 1 (see FIG. 6A). On the 4^{th} day after the treatment of a single dose of Construct 1, levels of IFN-γ, IL-12p40, IL-15, IFN-inducible protein (IP)-10, and TNFα increased in nearly all 9 patients in this analysis (see FIG. 6B).

The above changes in immune-stimulatory cytokine and chemokine levels are an early sign of natural killer (NK) and T cell activation. Furthermore, the balanced and physiological increase in systemic cytokine levels also indicate virus-induced immune activation. At the same time, the changes in cytokine levels were generally not associated with adverse events.

Direct IFN-γ ELISpot analysis was conducted on five patients, using samples from baseline and day 15 after administration of a single dose of Construct 1 or Construct 2, respectively. Specifically, cryopreserved and thawed peripheral blood mononuclear cells (PBMCs) from seven patients were stimulated with overlapping HPV16 E6/E7 peptides for 24 h (±2 h) for direct ex vivo IFN-γ ELISpot measurement. Enough cells were available from five of seven patients to be evaluated by ELISpot at the time of data cutoff. The number of circulating functional E6/E7-specific T cells in Construct 1- and Construct 2-treated patients reached levels that allowed detection in an ex vivo direct ELISpot (i.e., without in vitro expansion of T cells).

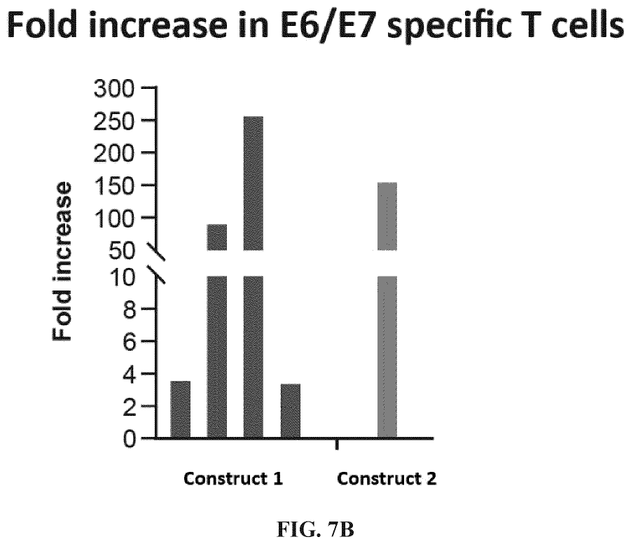

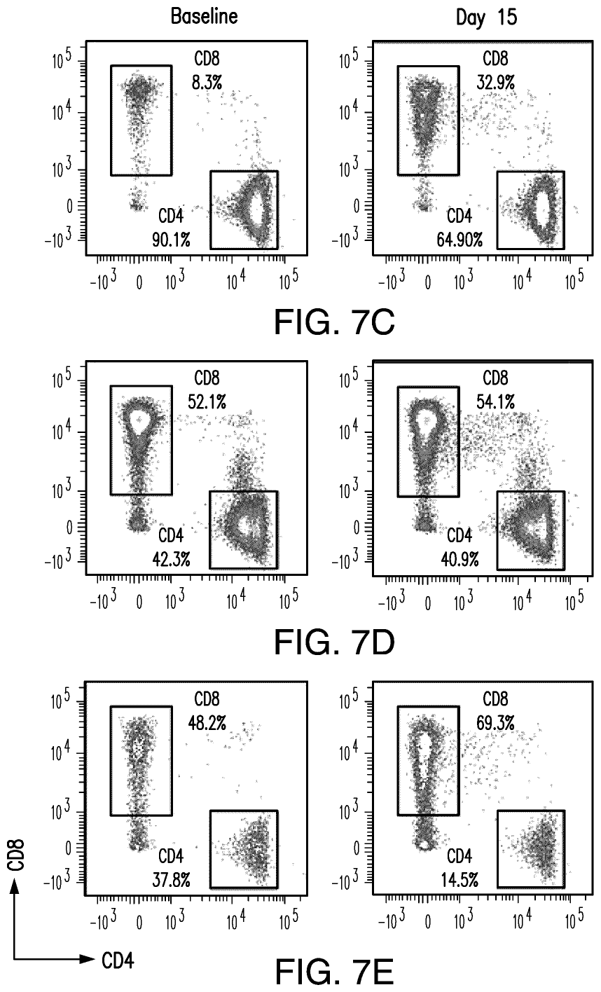

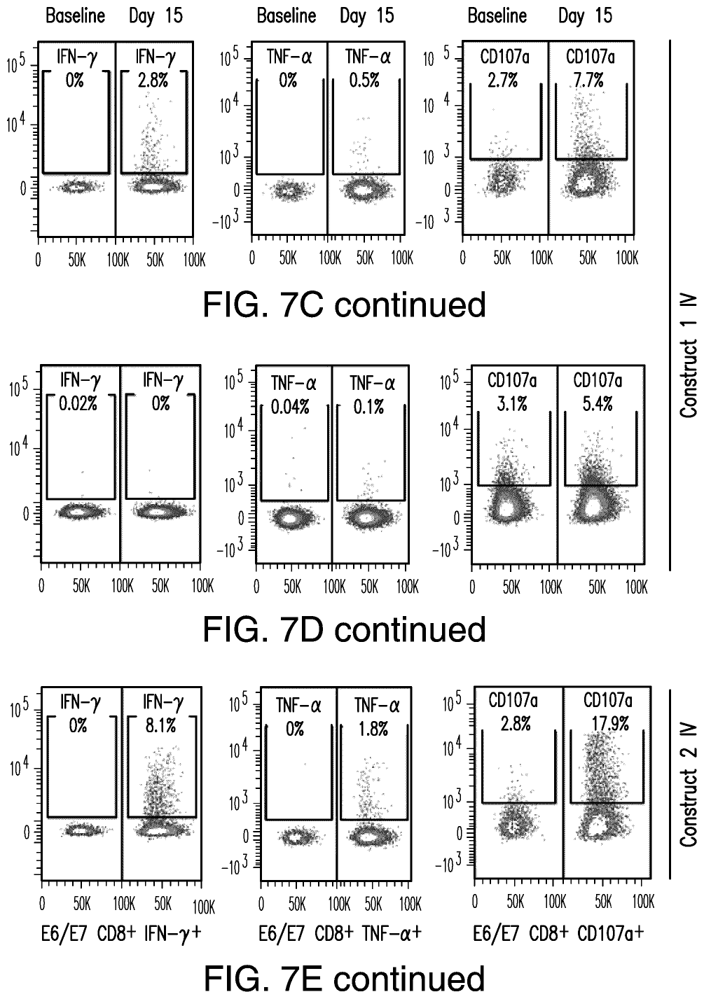

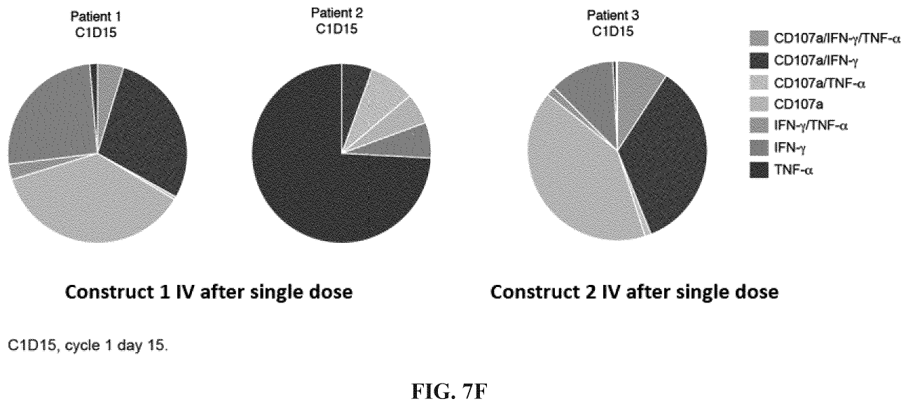

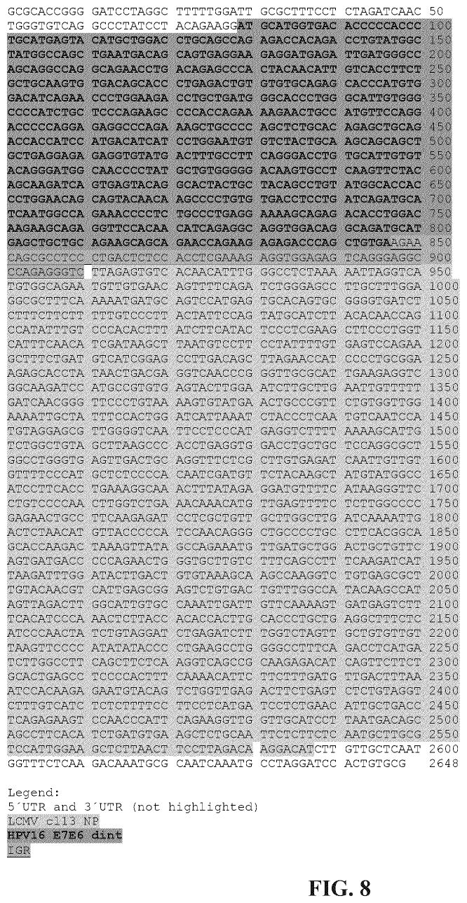

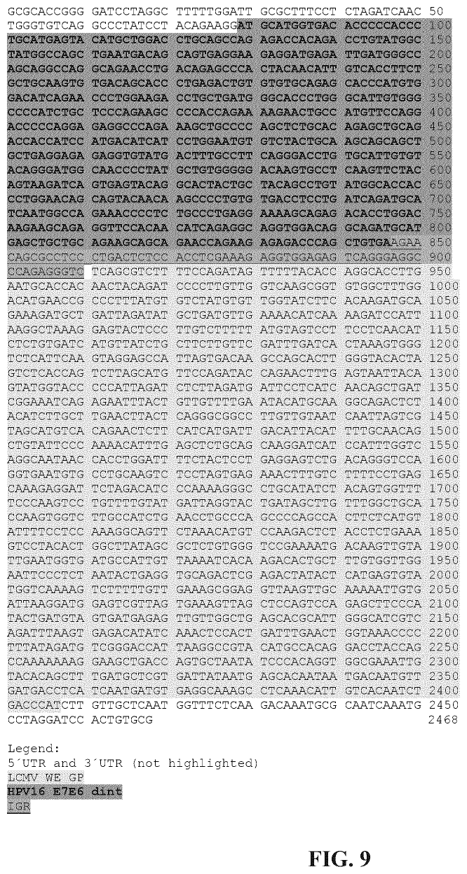

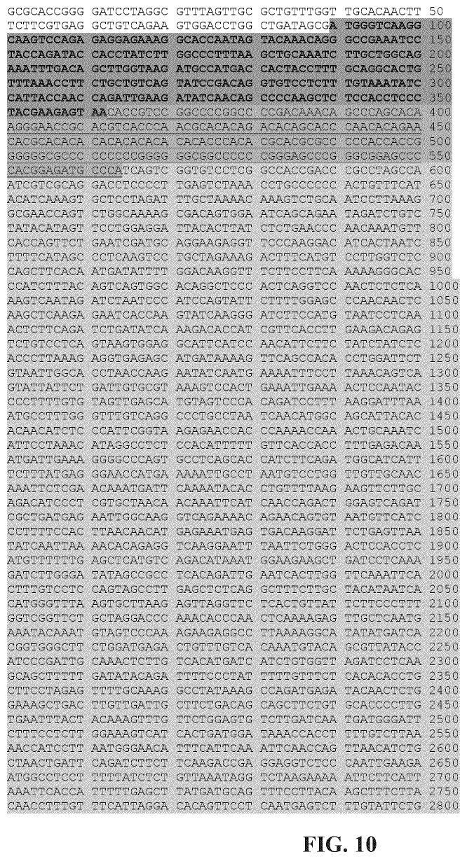

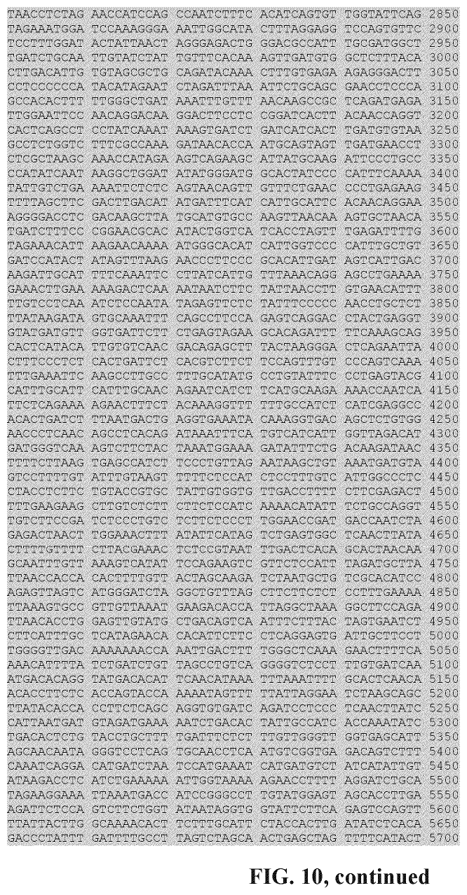

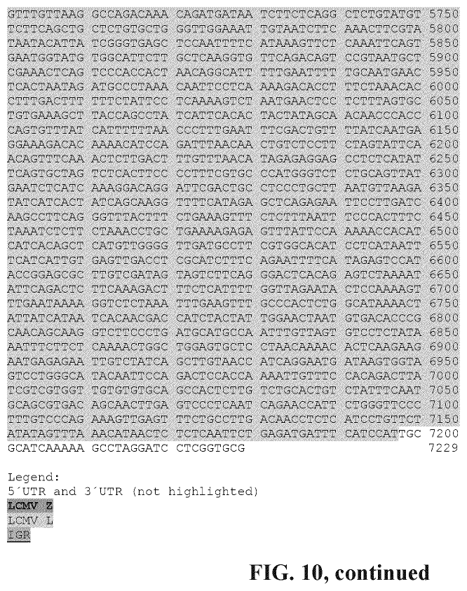

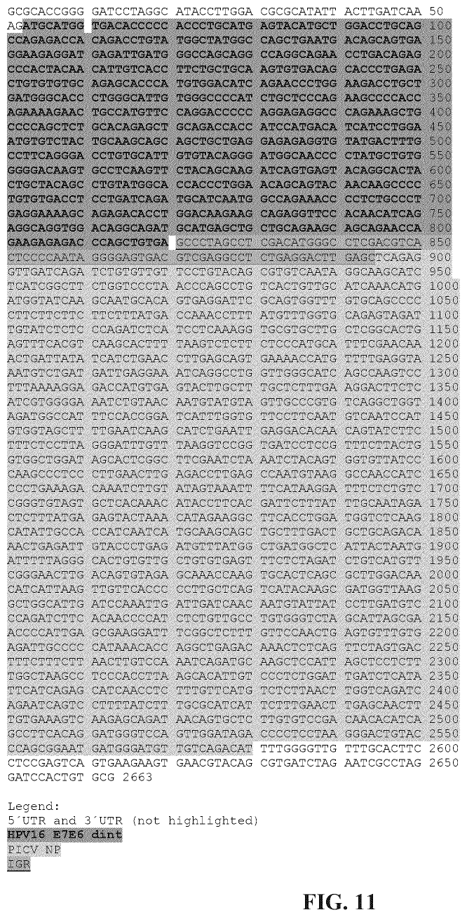

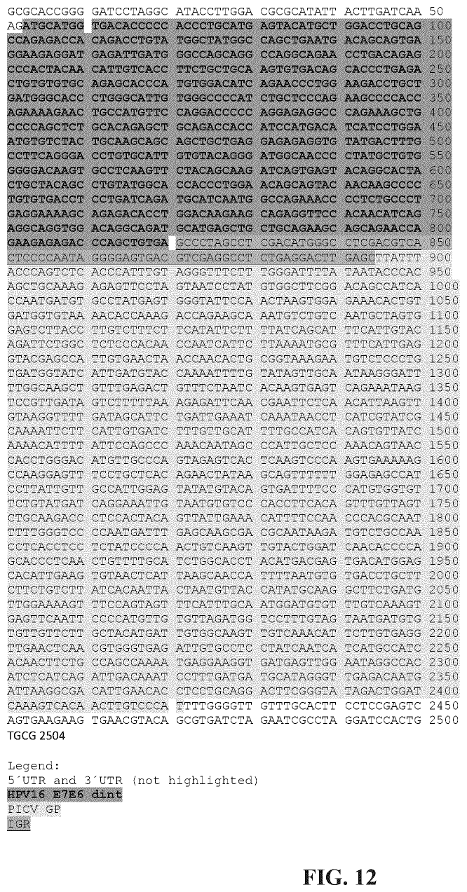

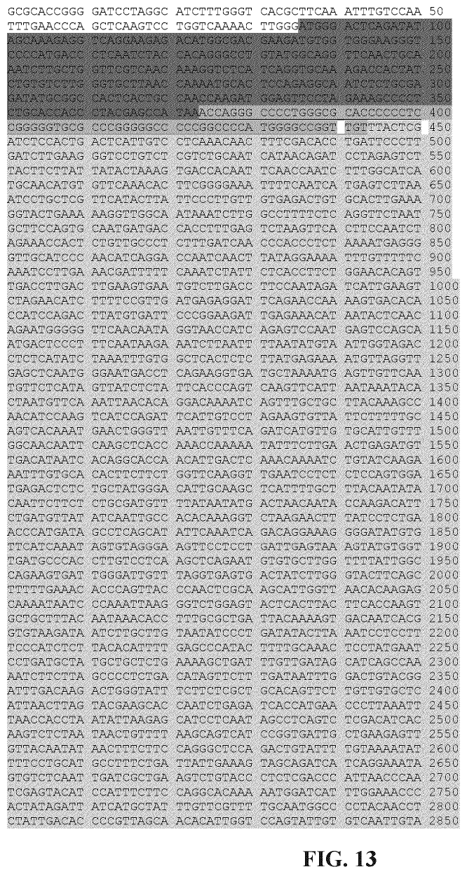

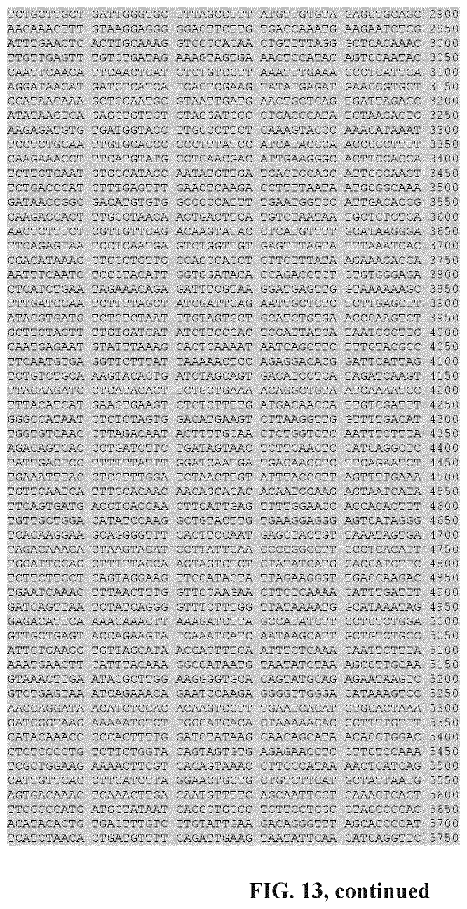

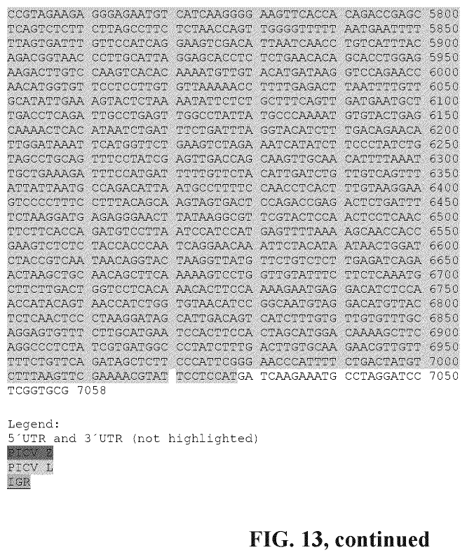

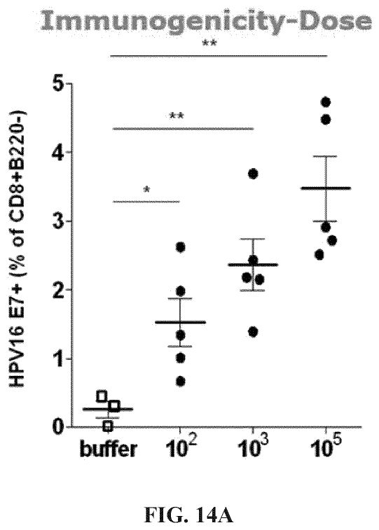

As shown in FIG. 7A, all patients (n=5) had a strong induction of antigen-specific T cell responses to HPV16 E6/E7 overlapping peptides from baseline to day 15. As shown in FIG. 7B, up to a 250-fold increase in antigen-specific IFN-γ—secreting T cells from baseline to day 15 was observed in four patients who received one dose of Construct 1 monotherapy systemically (IV). Additionally, a 150-fold increase was observed in one patient after administration of a single dose of Construct 2 monotherapy (IV).

Intracellular cytokine staining (ICS) was conducted on three patients. Specifically, samples from three patients were evaluated by ICS at baseline and day 15. Cryopreserved PBMCs from the three patients (two patients were treated with Construct 1 and one patient was treated with Construct 2) were stimulated with HPV16 E6/E7 overlapping peptides for 6 hours, and washed for subsequent immunostaining. The frequency of IFN-γ⁺, TNF-α⁺, IL-2⁺, CD107a⁺, CD4⁺, and CD8⁺ T cells was determined by ICS followed by multicolor flow cytometry analyzed. The three patients evaluated by ICS were among the seven patients whose cells were tested by ELISpot.

FIGS. 7C to 7E show representative pseudocolor plots with the frequencies of CD4⁺ and CD8⁺ T cells and frequencies of IFN-γ⁺, TNF-α⁺, and CD107a⁺ cells gated on CD8⁺ T cells at baseline and day 15 for the three patients. Two patients had an increase in T cells, predominantly CD8⁺ T cells, within the total peripheral T cell population after one dose of Construct 1 (8.3% vs 32.9%; see FIG. 7C) and Construct 2 (48.2% vs 69.3%; see FIG. 7E) at baseline versus day 15, respectively. Among CD8⁺ T cells, E6/E7-specific IFN-γ⁺ CD8⁺ T cells increased substantially following administration of single doses of Construct 1 or Construct 2. For example, antigen-specific IFN-γ⁺ CD8⁺ T cells increased from 0% at baseline to 2.8% on day 15 following a single dose of Construct 1 (see FIG. 7C). Following a single dose of Construct 2, antigen-specific IFN-γ⁺ CD8⁺ T cells increased from 0% at baseline to 8.1% on day 15 (see FIG. 7E). Similarly, E6/E7 specific CD8⁺ T cells had a higher expression of CD107a at day 15. On the other hand, one patient treated with Construct 1 had a slight increase in TNF-α⁺ and CD107a⁺, but no increase in IFN-γ⁺ or CD8⁺ T cells (see FIG. 7D).

Furthermore, to investigate the multi-functionality of the circulating HPV16 E6/E7-specific CD8⁺ T cells, co-staining of the degranulation marker CD107a and/or the cytokines IFN-γ and TNFα in the three patients was carried out, and the relative frequencies were depicted in FIG. 7F.

As illustrated above, the data from the first-in-human trial with arenavirus vectors demonstrated for the first time that patients with HPV16⁺ cancer who were injected systemically with E7/E6-expressing Construct 1 or Construct 2 as monotherapy had an increase in key systemic cytokine and chemokine levels, which is indicative of a virus-induced immune activation. In addition, the patients showed a strong induction of circulating HPV16 E6/E7-specific poly-functional CD8⁺ T cells up to 8% after the first dose.

Taken together, the example indicates arenavirus vectors expressing E7/E6 constitute a new potential therapy for patients with immunotherapy and/or chemotherapy-refractory HPV16⁺ cancers.

### Example III

Dose Escalation and Dose Expansion of Arenavirus-Based Cancer Immunotherapy in Patients with HPV16⁺ Cancers

This example describes a first-in-human Phase I/II, multinational, multicenter, open-label study of Construct 1 monotherapy and Construct 2/Construct 1 alternating 2-vector therapy in patients with HPV 16⁺ confirmed cancers. The example comprises two parts: Phase I Dose Escalation and Phase II Dose Expansion. Construct 1 monotherapy and/or Construct 2/Construct 1 alternating 2-vector therapy with or without pembrolizumab in patients with HPV 16⁺ head and neck squamous cell carcinoma (HNSCC) and other HPV 16⁺ confirmed cancers are explored during Dose Expansion.

1. Reagents Used and Previous In-Vivo Experiments

Construct 1 is a genetically engineered TheraT® vector based on the LCMV strain Clone 13 with the viral surface glycoprotein from LCMV strain WE (Kallert et al, 2017, Nat Commun. 2017; 8:15327). The Construct 1 vectors deliver a non-oncogenic synthetic fusion protein based on the HPV 16 E7 and E6 proteins (i.e., E7E6 fusion protein) in a tri-segmented, replication-competent, attenuated arenavirus vector. This synthetic E7E6 fusion protein has been particularly mutated in five pivotal positions (Cassetti et al, 2004, Vaccine. 2004; 22(3-4):520-27) to eliminate its retinoblastoma protein and tumor protein p53 binding abilities, thus abrogating the oncogenicity of the parental E7 and E6 proteins while still retaining full antigenicity.

Construct 1 contains several fractions of vector particles that contribute to immune response. To design Construct 1, TheraT® vectors were engineered to encode the mutated version of E7E6 such that one Small segment (S-segment) carries the LCMV viral surface glycoprotein from LCMV strain WE plus the mutated E7E6 fusion protein, and a second S segment carries the LCMV viral surface nucleoprotein plus a second, identical copy of the mutant E7E6 fusion protein. In addition, Construct 1 contains the Large segment (L-segment) of LCMV Clone 13. Thus, Construct 1 contains three genome segments (i.e., r3LCMV), two S segments, and one L segment. Inefficient packaging of the three genome segments is the basis of attenuation of Construct 1 compared to the wild type LCMV.

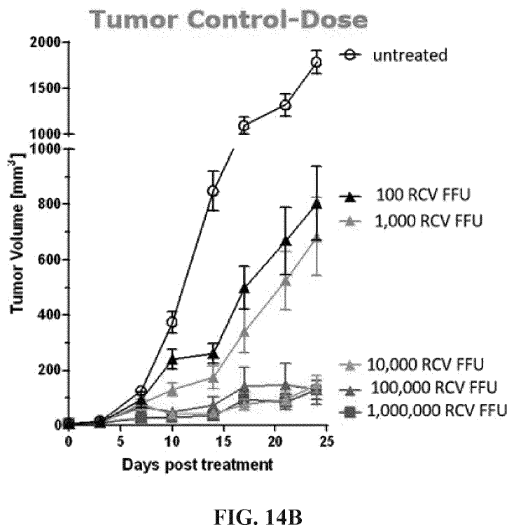

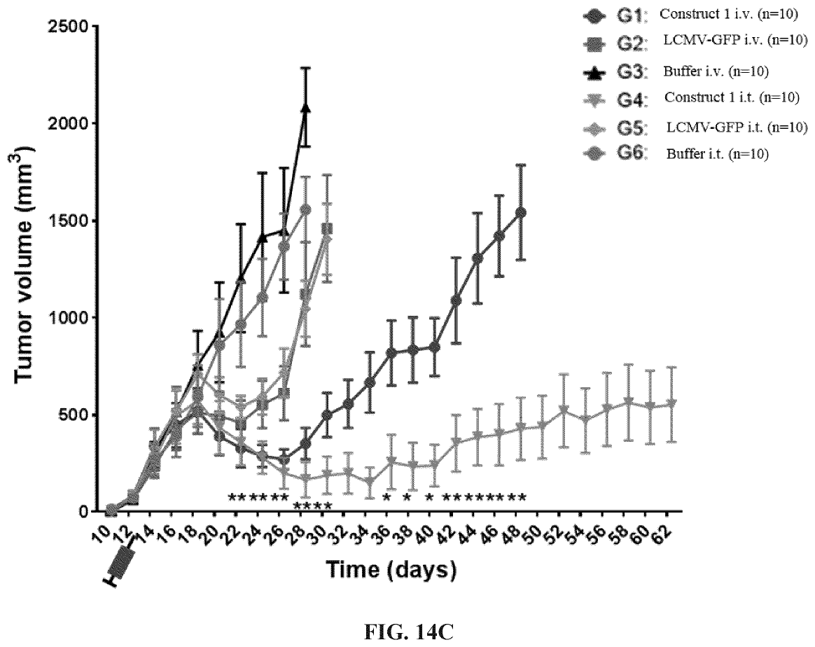

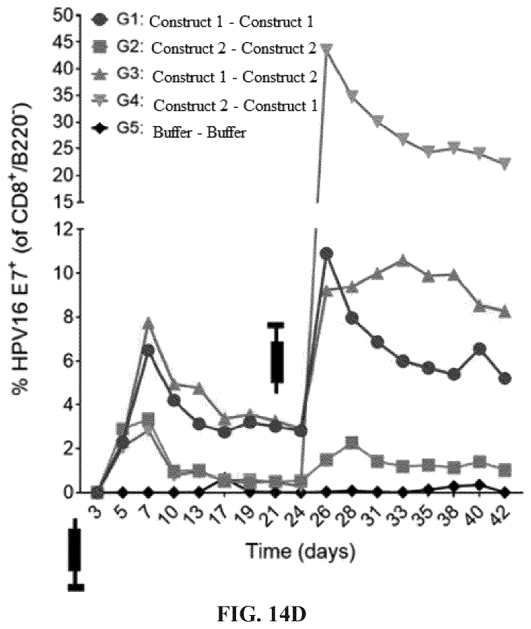

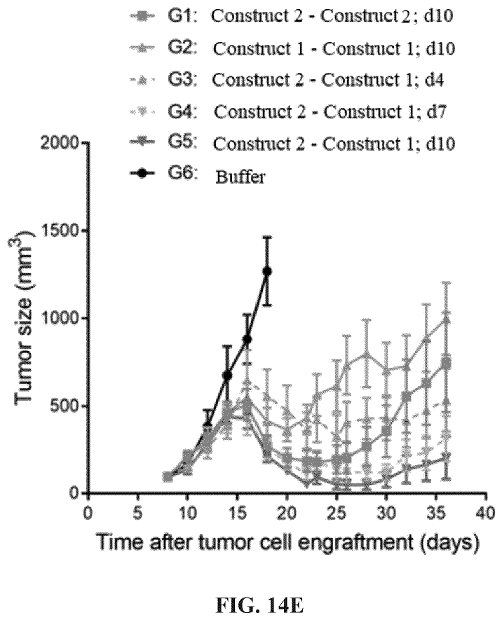

Nonclinical studies have demonstrated efficacy of Construct 1 in tumor control of palpable HPV 16⁺ tumors in the mouse TC-1 model. The dose of Construct 1 strongly correlated with immunogenicity, as depicted in FIG. 14A, and higher doses of Construct 1 also resulted in improvement of tumor growth control in the mouse TC-1 tumor model, as depicted in FIG. 14B. Specifically, Construct 1 doses containing as few as 100 RCV FFU significantly suppressed tumor growth. Dosing with the highest three doses of Construct 1, ranging from 10,000 to 1,000,000 RCV FFU per dose, led to almost complete suppression of tumor growth and significant benefit with regard to survival time and overall survival (OS). These data suggest that the maximal effective dose was reached at the lowest of those three doses (10,000 RCV FFU). All doses of Construct 1 were well tolerated in this model. In a further nonclinical study, Construct 1 was administered either IV or IT to animals when tumors were approximately 100 mm³. In both cases, as depicted in FIG. 14C, single doses of Construct 1 led to significant suppression of tumor growth in all treated mice, and IT administrations resulted in approximately 40% of long-term survival. This nonclinical data indicate that Construct 1 is highly efficacious when administered as local treatment of HPV 16⁺ tumors (IT administration of TC-1 tumors), which supports administration of Construct 1 by direct IT administration as part of the overall clinical development program. Furthermore, when these long term survivors were re-challenged with the same tumor 6 months later, no new tumor growth was detected. This protection was evident in mice that had only received a single dose of Construct 1 to treat the primary tumor. These results suggest the potential for further investigation into Construct 1 in humans for the treatment of primary tumors, metastatic tumors, and recurrent tumors.

Construct 2 is a genetically engineered, attenuated replication competent tri-segmented PICV vector based on the P18 variant of PICV. Construct 2 delivers the same non-oncogenic HPV 16 E7E6 antigens as those in Construct 1.

The Construct 2 vector was designed using the same tri-segment principle as that in Construct 1 vector by segregating the essential PICV viral surface glycoprotein and nucleoprotein from the original one genomic segment onto two artificially duplicated genomic S-segments. As a result, the Construct 2 vector contains three genome segments (i.e., r3PICV), including: one S segment carrying the PICV viral surface glycoprotein plus the mutated E7E6 fusion protein, a second S segment carries the PICV viral surface nucleoprotein plus a second, identical copy of the mutant E7E6 fusion protein, and an L-segment of P18 variant of PICV. Same as Construct 1, the genetic design of these S segments in Construct 2 absolutely prevents intersegmental recombination and reversion to a functional wild type-like single S segment encoding both PICV glycoprotein and nucleoprotein.

In the current example, Construct 2 is administered with Construct 1 following a sequential alternating IV administration strategy, in which Construct 2 is administered IV as the prime dose, the next dose is a Construct 1 IV booster dose, and the subsequent administrations alternate between Construct 2 and Construct 1 sequentially. This treatment plan is designated as “Construct 2/Construct 1 alternating 2-vector therapy.” Nonclinical studies using palpable HPV 16⁺ tumors in the TC1 model have demonstrated that the Construct 2/Construct 1 alternating treatment regimen resulted in suppression of tumor growth and prolonged overall survival (OS) that is superior to either vector alone (homologous Construct 1 & Construct 1 and Construct 2 & Construct 2) or prime with Construct 1 then boost with Construct 2 administrations. In addition, Construct 2/Construct 1 alternating 2-vector therapy induced the most potent HPV 16 E7-specific CD8 T cell responses (immunogenicity) among all the possible combination regimens tested. As depicted in FIG. 14D, among all rationally designed dosing regimens of Construct 1 and Construct 2, sequential IV administration of Construct 2 followed by Construct 1 (priming with 10⁵ RCV FFU of Construct 2 and boosting with 10⁵ RCV FFU of Construct 1, a regimen designated as Construct 2/Construct 1 alternating 2-vector administration) proved to be the most immunogenic regimen, which triggered an HPV 16, E7-specific CD8 T cell response substantially higher than those induced by other combination sequence or single vector regimens. Specifically, in the group receiving the Construct 2/Construct 1 alternating 2-vector regimen (G4), the frequencies of HPV E7-specific cells reached ˜43% of total CD8 T cells 5 days after the boost administration of Construct 1. Furthermore, the superior immunological effect of the Construct 2/Construct 1 alternating 2-vector therapy was sustained over the observation period (see FIG. 14D). In further efficacy studies using HPV 16⁺ TC 1 tumor model, as depicted in FIG. 14E, the Construct 2/Construct 1 alternating 2-vector therapy with IV administration of each vector both at a dose of 10⁵ RCV FFU (G4) also conferred superior tumor suppression capacity as compared to homologous prime-boost regimens using either Construct 1 or Construct 2 alone (G1 and G2). Together, these findings warrant further clinical testing of the Construct 2/Construct 1 alternating 2-vector therapy in HPV 16⁺ cancer patients.

In addition, pembrolizumab (KEYTRUDA®) is used in this example. Pembrolizumab has recently been approved by the FDA and the European Commission for the first-line treatment of patients with metastatic or unresectable recurrent HNSCC in monotherapy (for patients with tumors PD-L1 positive [CPS≥1]) or in combination with chemotherapy.

2. Experimental Design

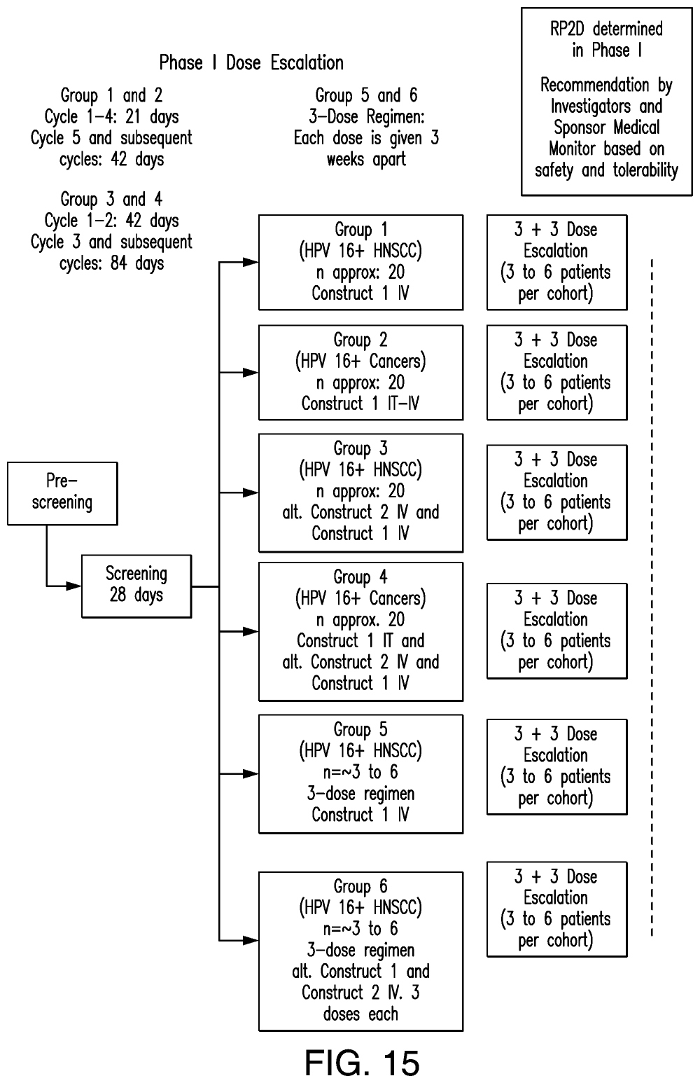

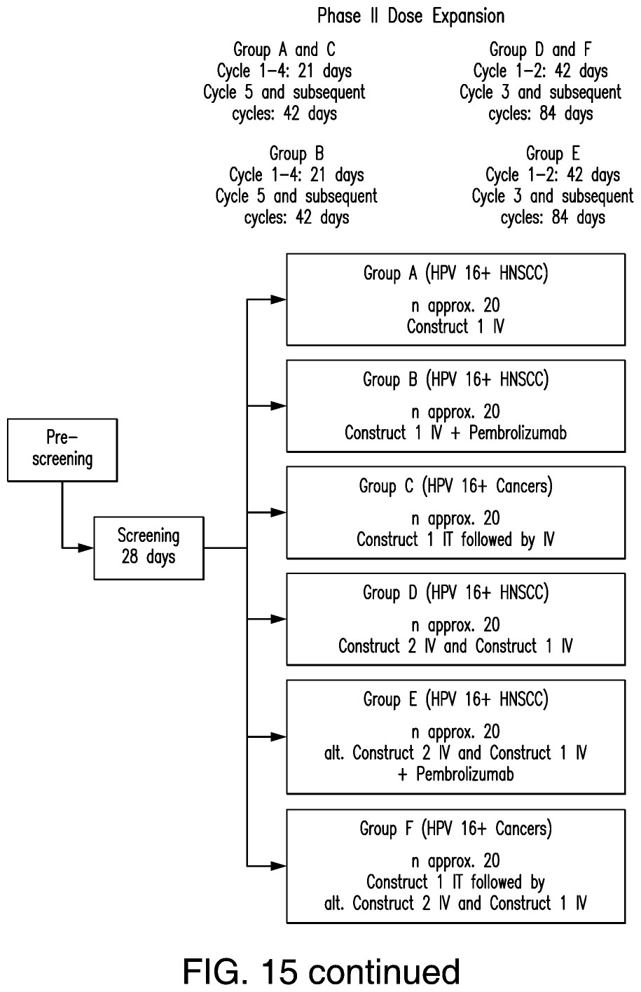

Patients with HPV 16⁺ HNSCC are enrolled in the IV treatment groups. Patients with HPV 16⁺ HNSCC and other HPV 16⁺ confirmed cancers are included in the IT IV treatment group. See FIG. 15 for a schematic of the study design.

The Phase I Dose Escalation has two treatment regimens: Construct 1 monotherapy and Construct 2/Construct 1 alternating 2-vector therapy. The Construct 1 monotherapy is given to 3 different groups (1, 2, and 5, see Table 2 below). Groups 1, 2, and 5 are studied to determine a safe recommended Phase II dose (RP2D) of Construct 1 for intravenous (IV) and intratumoral (IT) treatment. Group 5 explores Construct 1 as an IV administration given in 3 doses. The Construct 2/Construct 1 alternating 2-vector therapy is given to 3 different groups (3, 4, and 6). Groups 3, 4, and 6 are studied to determine a safe RP2D of Construct 2 for IV administration. Group 6 explores administering 3 doses of Construct 2/Construct 1 alternating 2-vector therapy such that patients receive Construct 2 administered first, followed by Construct 1, in an alternating manner until each patient has received 3 doses of Construct 2 and Construct 1 each, 6 doses in total.

The Phase II Dose Expansion have up to six treatment groups as shown in the Table 2 below. Based on the safety, efficacy, and/or biomarker data from the Dose Escalation results, the specific Dose Expansion Treatment Groups are opened accordingly. Phase II Dose Expansion Groups A and B commence upon completion of the Phase I Dose Escalation Group 1. Phase II Dose Expansion Groups D and E commence upon completion of the Phase I Dose Escalation Group 3. Phase II Dose Expansion Group C commence upon completion of the Phase I Dose Escalation Group 2. Phase II Dose Expansion Group F commence upon completion of the Phase I Dose Escalation Group 4.

For Phase I Dose Escalation, Groups 1, 2, 3, and 4, approximately 20 patients are enrolled in each group; Groups 5 and 6 enroll approximately 3 to 6 patients each. The actual number of patients enrolled in each group depend on when the RP2Ds are reached. Additional patients may be added in each group after a dose level has been determined to be safe to ensure sufficient biomarker data are obtained. For the Phase II Dose Expansion, Groups A, B, C, D, E, and F, approximately 20 patients are enrolled in each group. In total, approximately 200 patients are enrolled in this study, with approximately 100 in Phase I and approximately 60 to 100 in Phase II.

TABLE 2
Overview of Phase I & II Treatment Groups

| | Phase I dose escalation | | | | | | Phase II dose expansion | | | | | |
|---|---|---|---|---|---|---|---|---|---|---|---|---|
| Groups | 1 ^{a} | 2 ^{b} | 3 ^{c} | 4 ^{d} | 5 ^{a} | 6 ^{c} | A ^{a} | B ^{e} | C ^{b} | D ^{c} | E ^{f} | F ^{d} |
| Construct 1 | IV | IT- | — | — | — | — | IV | IV | IT- | — | — | — |
| monotherapy | | IV | | | | | | | IV | | | |
| Construct 2/ | — | — | IV- | IT- | — | — | — | — | — | IV- | IV- | IT- |
| Construct 1 | | | IV | IV | | | | | | IV | IV | IV |
| alternating 2- | | | | | | | | | | | | |
| vector therapy | | | | | | | | | | | | |
| Construct 1 | — | — | — | — | IV | — | — | — | — | — | — | — |
| 3-dose | | | | | | | | | | | | |
| treatment | | | | | | | | | | | | |
| Construct 2/ | — | — | — | — | — | IV- | — | — | — | — | — | — |
| Construct 1 3- | | | | | | IV | | | | | | |
| dose treatment | | | | | | | | | | | | |
| Pembrolizumab | — | — | — | — | — | — | — | IV | — | — | IV | — |

IT = intratumoral, IV = intravenous(ly).
^{a} Construct 1 as an IV administration.
^{b} Construct 1 as an IT administration for the first dose, followed by Construct 1 as an IV administration in the subsequent doses.
^{c} Sequential alternating IV administrations of Construct 2 and Construct 1.
^{d} First dose is with IT administration of Construct 1, followed by sequential alternating IV administrations of Construct 2 and Construct 1.
^{e} Construct 1 as an IV administration in combination with pembrolizumab.

For Groups 2, 4, C, and F, where first dose is with IT administration of Construct 1, if IT administration should not be administered, the first dose is then be given as IV for the given dose level. In this case, this group for the given dose level is limited to HPV 16⁺ non-HNSCC cancers. ^{f} Sequential alternating administrations of Construct 2 IV and Construct 1 IV, and pembrolizumab.

2.1 Phase I Dose Escalation

Phase I Dose Escalation is a safety and tolerability phase; there is no primary efficacy endpoint. Incidence of dose-limiting toxicity (DLTs) from the first study drug administered during the DLT observation period is monitored. Safety parameters (e.g., types, frequency, and severity of AEs and SAEs) are recorded. Tolerability parameters (e.g., dose interruptions, reductions and dose intensity, and evaluations of laboratory values) are also recorded. The secondary efficacy endpoints for Phase I are objective response rate (ORR), and disease control rate (DCR) Response Evaluation Criteria in Solid Tumors (RECIST) and immune Response Evaluation Criteria in Solid Tumors (iRECIST). The ORR and DCR are presented. Time to event efficacy endpoints (duration of response, progression free survival [PFS], and overall survival [OS]) are listed. As exploratory readouts, E7 and E6 antigen-specific T cell response, CD4 and CD8 T cell measurements, changes in SUV-based quantitative measures on CD8 tracer PET scan at baseline and post-treatment, and biomarkers in tumor specimens, blood, and serum/plasma are tested. The six groups of patients are assigned and treated as below.

Group 1 (Construct 1 IV only): Construct 1 as an ongoing IV administration in patients with HPV 16⁺ HNSCC.

Group 2 (Construct 1 IT-IV): Construct 1 as an IT administration for the first dose, followed by ongoing Construct 1 as an IV administration for the subsequent doses in patients with HPV 16⁺ cancers with a safe and accessible tumor site amenable for IT administration.

Group 3 (Construct 2 IV and Construct 1 IV): Construct 2 as an IV administration (initial) and then followed by Construct 1 IV, alternating treatment on an ongoing basis in patients with HPV 16⁺ HNSCC.

[symbol] 1 IT, followed by Construct 2 IV and Construct 1 IV): Construct 1 as an IT administration for the first dose. Treatment is followed by Construct 2 IV, then followed by Construct 1 IV, alternating treatment on an ongoing basis in patients with HPV 16⁺ cancers with a safe and accessible tumor site amenable for IT administration. Only one tumor site for IT administration required.

Group 5 (3 doses of Construct 1 IV): Assess if 3 administrations at the highest doses evaluated induce a similar immunogenicity to E7/E6 than continuous dosing. The patient would have an option to receive another 3 doses of Construct 1 IV if they progressed radiologically.

Group 6 (3 doses of Construct 2 IV and Construct 1 IV each): Assess 3 administrations of Construct 1 and Construct 2 each, for a total of 6 administrations, in order to test if a limited number of administrations of the construct 1 and 2 therapeutic vaccines permit to reach a level of circulating T cells compatible with control of the disease and lasting after the last injection.

Patients from groups 1, 3, 5, and 6 are the ones who have HPV 16⁺ HNSCC with tumor progression or recurrence on standard of care therapy, including more than or equal to 1 systemic therapy. Patients from groups 2 and 4 are the ones who have HPV 16⁺ cancers with a safe and accessible tumor site amenable for IT administration, who had tumor progression or recurrence on standard of care therapy, including more than or equal to 1 systemic therapy.

During Phase I Dose Escalation, Backfill cohorts are explored to enroll additional patients to be evaluated at the dose level(s) of Construct 1 monotherapy and/or Construct 2/Construct 1 alternating 2-vector therapy that is declared safe to better assess safety and potential efficacy.

Selected backfill cohorts require “fresh paired biopsies” for the purpose of investigating effects of Construct 1 and/or 2 treatment on molecular signaling and tumor cell responses, identifying biomarkers that may be predictive of efficacy and response. In addition, tumor material is used to quantify the levels of tumor infiltrated lymphocytes by immunohistochemistry staining. The analysis provides assessment of tumor infiltration of immune cells and particularly CD8⁺ T cells in the tumor.

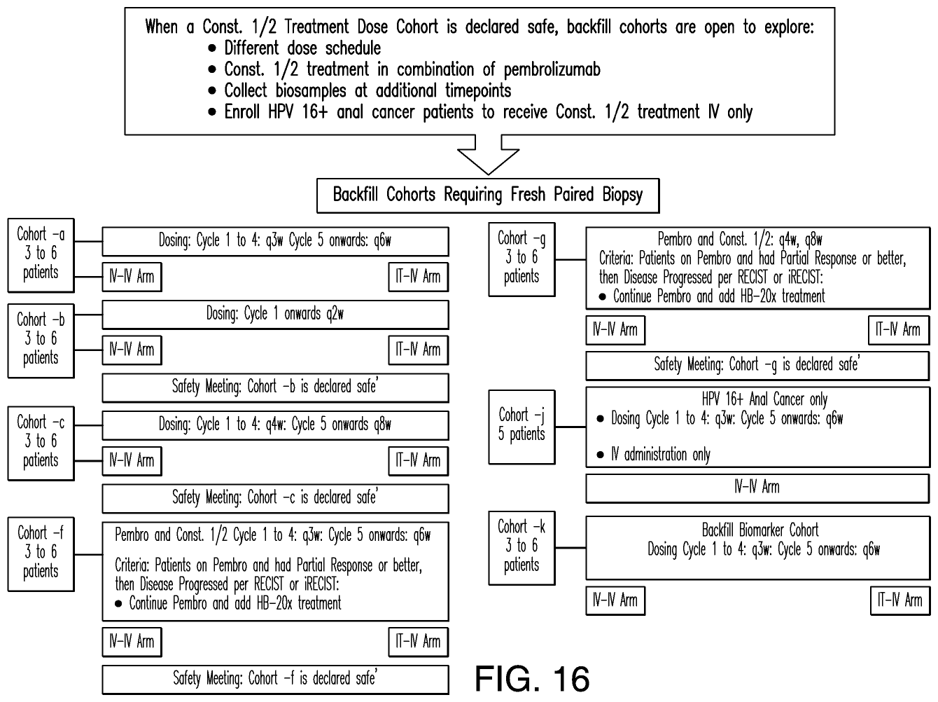

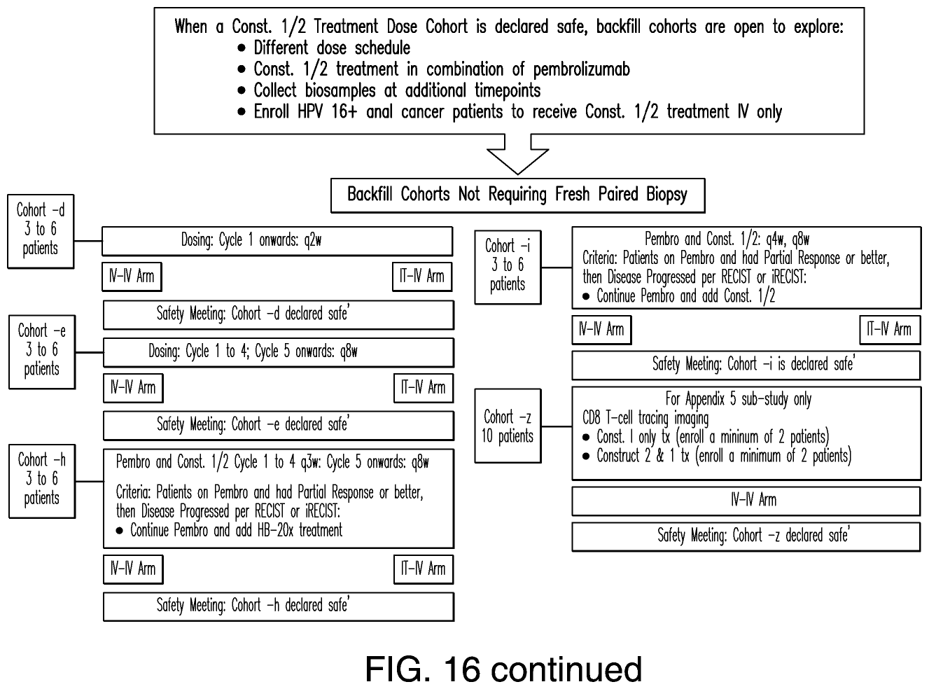

A schematic of the backfill cohorts is presented in FIG. 16. Backfill cohorts keep the same number as the Construct 1 monotherapy dose cohort number and are further identified by the addition of a lowercase letter for each backfill cohort. As an example, after Construct 1 monotherapy dose Cohort 1 exploring Construct 1 at dose level 1 is declared safe, the backfill cohorts are named as Cohorts 1a, 1b, 1c, 1d, and 1e.

The backfill cohorts may explore Construct 1 and/or Construct 2 treatment:
at a different administration schedule,
to explore the combination of pembrolizumab with Construct 1 and/or Construct 2 treatment,
to enroll HPV 16+ anal cancer patients to administer Construct 1 and/or Construct 2 as an IV only, and/or
to collect biosamples at additional timepoints for central analysis of viral shedding, lymphocyte subsets (TruCount), serum biomarker, and immunogenicity.

2.1.1 Additional Biomarker Analysis

For backfill cohort -k, additional timepoints for collection of viral shedding, lymphocyte subsets (TruCount), serum biomarker, and immunogenicity are added to provide additional translational and biomarker data for Construct 1 monotherapy or Construct 2/Construct 1 alternating 2-vector therapy at the q3w, q6w dose schedule.

2.1.2 Exploration of Alternate Dosing Schedule for Construct 1 and/or 2 Study Treatment

The frequency of dosing using the previously recommended safe dose is increased. For example, backfill cohorts -b and -d explore Construct 1 monotherapy in an every 2 week dose (q2w) administration schedule. Backfill cohorts -c and -e explore Construct 1 monotherapy and/or Construct 2/Construct 1 alternating 2-vector therapy at a dosing schedule of every 4 weeks from Cycles 1 to 4, and every 8 weeks starting on Cycle 5. This schedule is known as “q4w, q8w.” If “q2w” or “q4w, q8w” dosing schedule in the backfill cohorts are opened, DLTs are evaluated using the same process as the Dose Escalation cohorts. Characterization of safety, tolerability, antitumor activity, and immunogenicity of Construct 1 and/or 2 study treatment is evaluated in the alternate dosing schedule explored.

2.1.3 Patients on Treatment with Pembrolizumab for Whom Construct 1 and/or 2 Study Treatment is Added Upon Progression

In the Phase I portion of the study, selected backfill cohorts enroll patients on treatment with pembrolizumab monotherapy who have since had disease progression to continue with their pembrolizumab treatment and add either a Construct 1 monotherapy or Construct 2/Construct 1 alternating 2-vector therapy. The rationale for allowing patients to continue their pembrolizumab monotherapy after they had disease progression and to add a Construct 1 and/or 2 treatment is to explore the hypothesis that once patients progress on pembrolizumab, those patients who are resistant or refractory may start responding again if pembrolizumab is administered in combination with another treatment.

Backfill cohorts -f and -h allow patients on pembrolizumab treatment who have since had disease progression to continue with their pembrolizumab and add Construct 1 monotherapy or Construct 2/Construct 1 alternating 2-vector therapy following the “q3w, q6w” schedule. Similarly, backfill cohorts -g and -i allow patients on pembrolizumab treatment who have since had disease progression to continue with their pembrolizumab and add Construct 1 monotherapy or Construct 2/Construct 1 alternating 2-vector therapy following the “q4w, q8w.”

Patients' disease progression while on pembrolizumab monotherapy should be characterized as having refractory disease or resistance to pembrolizumab accordingly. The definitions of refractory and resistant disease are: Patients with refractory disease (primary resistance) are defined as having progressed within <6 months of the first dose of pembrolizumab monotherapy by Response Evaluation Criteria in Solid Tumors (RECIST) version 1.1 (v1.1). A patient who presents with stable disease (SD) and then meets criteria for disease progression within <6 months of the first dose of pembrolizumab, should be considered as having refractory disease. Patients with resistance to pembrolizumab (secondary resistance) are defined as having progressed ≥6 months after the first dose of pembrolizumab monotherapy. Exception: patients who have disease progression after discontinuation due to AEs, and who did not receive at least 6 months of pembrolizumab and had no evidence of initial clinical benefit would be best classified as having primary resistance.

2.1.4 HPV 16⁺ Anal Cancer Patients Receiving IV Administration Only

Backfill cohort -j enrolls five HPV 16⁺ anal cancer patients receiving IV administration only to evaluate the efficacy of either Construct 1 monotherapy or Construct 2/Construct 1 alternating 2-vector therapy at the q3w, q6w dose schedule. To be eligible to participate in this backfill cohort(s), patients must meet the required inclusion and exclusion criteria.

2.2 Phase II Dose Expansion

The Phase II Dose Expansion assesses Construct 1 monotherapy and/or Construct 2/Construct 1 alternating 2-vector therapy at the RP2D that has been defined in the Phase I Dose Escalation.

The patients enrolled in groups A, B, D, and E are the ones with HPV 16⁺ HNSCC with tumor progression or recurrence on standard of care therapy, including more than or equal to 1 systemic therapy. The patients enrolled in groups C and F are the ones with HPV 16⁺ cancers with a safe and accessible tumor site amenable for IT administration, who had tumor progression or recurrence on standard of care therapy, including more than or equal to 1 systemic therapy.

The primary efficacy endpoints in the Phase II Dose Expansion groups are the ORR and disease control rate based on RECIST and iRECIST. The ORR by RECIST are summarized using the point estimate together with the exact two-sided 95% CIs according to the Clopper-Pearson method. The secondary efficacy endpoints for the Phase II are the duration of response, PFS and OS. Safety parameters (e.g., types, frequency, and severity of AEs and SAEs) are recorded. Tolerability parameters (e.g., dose interruptions, reductions and dose intensity, and evaluations of laboratory values) are also recorded. The percentage change in target lesion tumor size from baseline is summarized using descriptive statistics and presented at each timepoint. Best percentage change in tumor size are also summarized. Tumor size is also presented graphically using waterfall plots. Two patients within a treatment group with objective responses are viewed as supportive of further development. As exploratory readouts, E7 and E6 antigen-specific T cell response, CD4 and CD8 T cell measurements, and biomarkers in tumor specimens, blood, and serum/plasma are tested.

3. Patient Population

This example is conducted in adult patients with: HPV 16⁺ HNSCC and HPV 16⁺ cancer of any origin (e.g., cervical, anal, vaginal, vulvar, or penile cancers). Only patients meeting all the inclusion criteria and none of the exclusion criteria may be enrolled into the study. The below criteria apply to both Phase I Dose Escalation and Phase II Dose Expansion.

3.1 Inclusion Criteria

Patients are eligible to be included in the study only if all the following criteria apply:
1. Male or female patients 18 years of age, or older, at the time of signing the Informed Consent Form (ICF).
2. Patient must have ≥1 measurable lesion by computed tomography (CT) and/or magnetic resonance imaging (MRI), that is assessed for tumor response following RECIST and iRECIST during study conduct.
3. Eastern Cooperative Oncology Group (ECOG) performance status of 0 to 1.
4. Prior curative radiation therapy must have been completed ≥4 weeks prior to study treatment administration. Prior focal palliative radiotherapy must have been completed ≥2 weeks prior to study treatment administration.
5. Screening laboratory values must meet the following criteria and should be obtained within 28 days prior to study treatment administration:
   Absolute neutrophil count ≥1,500/mm³ (1.5×10⁹/L).
   Platelets ≥100×10³/mm³ (100×10⁹/L).
   Hemoglobin ≥9 g/dL.
   Serum creatinine ≤2.0×upper limit of normal (ULN) or creatinine clearance >30 mL/min (using the Cockcroft-Gault formula).
   Aspartate aminotransferase and alanine aminotransferase ≤2.5×ULN or ≤5×ULN for subjects with liver metastases.
   Total bilirubin ≤1.5×ULN. Direct bilirubin ≤ULN for subjects with total bilirubin levels >1.5×ULN.
   International Normalized Ratio (INR) or Prothrombin Time (PT)≤1.5×ULN unless subject is receiving anticoagulant therapy as long as PT or partial thromboplastin time (PTT) is within therapeutic range of intended use of anticoagulants.
   Activated Partial Thromboplastin Time (aPTT) or Partial Thromboplastin Time (PTT)≤1.5×ULN unless subject is receiving anticoagulant therapy as long as PT or PTT is within therapeutic range of intended use of anticoagulants.
6. Able to understand and willing to comply with study procedures, restrictions, and requirements, in the opinion of the Investigator.
7. Willing and able to give voluntary informed consent for participation in the study.

For Patients Enrolled in Treatment Group 1, Group 3, Group 5, Group 6, Group A, or Group D
8. Patient must have documentation of confirmed HPV 16⁺ HNSCC via genotype testing.
9. Patient must have had tumor progression or recurrence on standard of care therapy, including ≥1 systemic therapy, (e.g., failed platinum-based therapy and/or anti-PD-1/anti-PD-L1 therapy) or be a patient for whom standard of care therapy is contraindicated.
10. Tumor tissue (archival [no older than 2 years] or able to provide fresh biopsy specimen during Screening) collected following the patient's progression from the last treatment, unless agreed otherwise between the Sponsor and the Investigator.

For Patients Enrolled in Backfill Cohorts to Add Construct 1 and/or 2 Treatment to Their Ongoing Pembrolizumab Therapy (applicable to backfill cohorts -f, -g, -h, and -i) in Phase I Treatment Groups 1, 2, 3, or 4
11. Patient must have documentation of confirmed HPV 16⁺ HNSCC via genotype testing.
12. Patient must have had tumor progression or recurrence on standard of care therapy, including ≥1 systemic therapy, (e.g., failed platinum-based therapy and/or anti-PD-1/anti-PD-L1 therapy) or be a patient for whom standard of care therapy is contraindicated.
13. Tumor tissue (archival [no older than 2 years] or able to provide fresh biopsy specimen during Screening) collected following the patient's progression from the last treatment, unless agreed otherwise between the Sponsor and the Investigator.
14. Patients must be receiving ongoing treatment with pembrolizumab monotherapy prior to enrollment and show evidence of progression after initial response.
   The following criteria must be all met.
   Patients must:
      Have received at least 2 cycles of pembrolizumab monotherapy on a q3w schedule or equivalent for a longer schedule (e.g., 1 cycle if a q6w schedule)
      Present with best response of either tumor response (complete response [CR] or partial response [PR]) or prolonged SD lasting 6 months or greater as per RECIST v1.1 during pembrolizumab monotherapy
      Have then progressed on pembrolizumab monotherapy by iRECIST (progression confirmed by a second scan at least 4 weeks after initial RECIST v1.1 progression, if clinically acceptable).
   Note: Patients must NOT have discontinued pembrolizumab monotherapy prior to starting this study.

For Patients enrolled in Phase I Backfill Cohort -j (HPV 16⁺ Anal Cancer)

Patients must have:
15. Histologically or cytologically confirmed locally advanced or metastatic squamous cell carcinoma of the anal canal.
   Note: Patients HIV positive who do not meet acquired immunodeficiency syndrome (AIDS) criteria are eligible. Note: Patients who may have received the HPV preventive vaccine more than 90 days prior to enrollment are eligible.
16. Documented HPV 16⁺ tumor by immunohistochemistry. Sample for HPV 16⁺ testing must be submitted prior to enrollment for genotype testing.
17. Tumor progression or recurrence on standard of care treatment, including ≥1 systemic therapy such as platinum-based chemotherapy, or be ineligible for standard of care therapy.
18. Safe and accessible tumor site amenable for biopsy, unless agreed otherwise between the Sponsor and the Investigator:
   Note: Tumor tissue may be obtained by image-guided biopsy, such as interventional radiology, according to the institution's own guidelines and requirements for such procedures.
19. ≥1 measurable lesion, which is assessed for tumor response following RECIST and iRECIST during study conduct, apart from the tumor site(s) amenable for biopsy.

For Patients Enrolled in Treatment Group 2, Group 4, Group C, or Group F (First Dose is Construct 1 Given as an Intratumoral Administration)
20. Documentation of confirmed HPV 16⁺ cancer (of any origin) via genotype testing.
21. Patients who have had tumor progression or recurrence on standard of care therapy, including ≥1 systemic therapy, or for patients for whom standard of care therapy is contraindicated.
22. Patient must have a safe and accessible tumor site amenable for biopsy and IT administration, unless agreed otherwise between the Sponsor and the Investigator:
   Tumor tissue may be obtained by image-guided biopsy, such as interventional radiology, according to the institution's own guidelines and requirements for such procedures.
   IT Construct 1 is then administered to the tumor site.
23. Apart from the tumor site(s) amenable for biopsy and IT administration, the patient must have ≥1 measurable lesion, that is assessed for tumor response following RECIST and iRECIST during study conduct.

For Patients Enrolled in Treatment Group 2, Group 4, Group C, or Group F (if intratumoral Administration is Given as an IV Administration Instead)
24. Documentation of confirmed HPV 16⁺ non-HNSCC cancer via genotype testing.
25. Patients who have had tumor progression or recurrence on standard of care therapy, including ≥1 systemic therapy, or for patients for whom standard of care therapy is contraindicated.
26. Tumor tissue (archival [no older than 2 years] or able to provide fresh biopsy specimen during Screening) collected following the patient's progression from the last treatment, unless agreed otherwise between the Sponsor and the Investigator.

For Patients Enrolled in Phase II Treatment Group B or Group E
27. Documentation of confirmed HPV 16⁺ HNSCC via genotype testing.
28. Patient must be eligible, as per package insert or Summary of Product Characteristics (SmPC), to receive pembrolizumab (i.e., naïve to anti-PD-1/anti-PD-L1 therapy and have progressed on platinum-based therapy).
29. Tumor tissue (archival [no older than 2 years] or able to provide fresh biopsy specimen during Screening) collected following the patient's progression from the last treatment, unless agreed otherwise between the Sponsor and the Investigator.

3.2 Exclusion Criteria

Patients are excluded from the study if the patient meets or has any of the following criteria:

All Patients
1. Patients with untreated and/or symptomatic metastatic central nervous system (CNS) disease and/or carcinomatous meningitis.
   Exception: patients with brain/CNS metastases are eligible if:
      They have undergone surgery or radiotherapy, and their disease is stable (without evidence of progression by imaging using the identical imaging modality for each assessment, either MRI or CT scan), and any neurologic symptoms have returned to Baseline, AND
      they have no evidence of new or enlarging brain metastases, AND
      they have been on a stable dose of corticosteroids (≤10 mg prednisone or equivalent) for ≥4 weeks prior to the first administration of study treatment is eligible.
   Note: This exception does not include carcinomatous meningitis which is excluded regardless of clinical stability.
2. Any serious or uncontrolled medical disorder that may increase the risk associated with study participation or study treatment administration, impair the ability of the patient to receive study treatment, or interfere with the interpretation of the study results. This includes clinically significant (i.e., active) cardiovascular disease, including cerebral vascular accident/stroke and myocardial infarction less than 6 months prior to enrollment, unstable angina, congestive heart failure (New York Heart Association Classification Class II), or serious uncontrolled cardiac arrhythmias.
3. Concurrent malignancy that is clinically significant or requires active intervention at the time of Screening (with the exception of adequately treated, basal or squamous cell carcinoma, non-melanomatous skin cancer), unless agreed otherwise between the Sponsor and the Investigator.
4. Active, known or suspected, autoimmune or inflammatory disorders requiring immunosuppressive therapy, with the exception of low-dose prednisone (≤10 mg or equivalent). The following are exceptions to this criterion:
   Patients with vitiligo or alopecia.
   Patients with hypothyroidism (e.g., following Hashimoto syndrome) stable on hormone replacement.
   Any chronic skin condition that does not require systemic treatment.
5. Toxicity attributed to systemic prior anticancer therapy, including radiation and surgery, other than alopecia and fatigue that has not resolved to Grade 1 or Baseline prior to the first administration of study treatment. Patients with toxicities attributed to systemic prior anticancer therapy, which are not expected to resolve and result in long lasting sequelae, such as neuropathy or ototoxicity after platinum-based therapy, are permitted to enroll.
6. Treatment with any chemotherapy, biological, or investigational therapy for cancer within 28 days of the first administration of study treatment, unless agreed otherwise between the Sponsor and the Investigator on a case-by-case basis based on the half-life of the anticancer therapy.
   Exception: Ongoing treatment with pembrolizumab is permitted if the subject is enrolling in a backfill cohort continuing pembrolizumab and adding either Construct 1 monotherapy or Construct 2/Construct 1 alternating 2-vector therapy.
7. Treatment with immunosuppressive or replacement medication:
   Immunosuppressive doses of systemic medication, such as steroids or absorbed topical steroids (doses >10 mg/day prednisone or equivalent), within 14 days of the first administration of study treatment.
   Note: inhaled or topical steroids and adrenal replacement in doses equivalent to >10 mg/day prednisone are permitted in the absence of active autoimmune disease.
   Any chronic immunosuppressive medication within 6 months prior to the first administration of study treatment (unless agreed otherwise between the Sponsor and the Investigator on a case-by-case basis).
   Active autoimmune disease that has required systemic treatment in past 2 years (i.e., with use of disease modifying agents, corticosteroids, or immunosuppressive drugs). Replacement therapy (e.g., thyroxine, insulin, or physiologic corticosteroid replacement therapy for adrenal or pituitary insufficiency, etc.) is not considered a form of systemic treatment.
8. Prior anaphylactic or other severe reaction to human immunoglobulin or antibody formulation administration.
9. Live vaccines received within 28 days prior to the first dose of study treatment, unless agreed otherwise between the Sponsor and Investigator.
10. Herbal remedies with immune-stimulating properties or known to potentially interfere with major organ function taken within 28 days prior to the first dose of study treatment, unless agreed otherwise between the Sponsor and Investigator.
11. Female patients who are pregnant, breastfeeding, or plan on becoming pregnant during the study.
12. Active infection requiring systemic therapy, unless agreed otherwise between the Sponsor and Investigator on a case-by-case basis.
13. Positive test for hepatitis B surface antigen (HBsAg) or hepatitis C virus (HCV) antibody, indicating acute or chronic infection. Patients who test positive for HCV antibody but negative for HCV ribonucleic acid (RNA) are permitted to enroll.
14. Known history of AIDS. Testing for the HIV is not mandatory.
   Note: Patients HIV-positive patients with CD4 T cells >200/mm³ who do not have AIDS are eligible.
15. Other concurrent severe and/or uncontrolled medical conditions that would, in the Investigator's judgment, contraindicate participation in this study (e.g., pancreatitis, active hepatitis, chronic obstructive or restrictive pulmonary disease including dyspnea at rest or interstitial lung disease, adrenal insufficiency, uncontrolled hypertension).
16. Psychological, familial, sociological, or geographical conditions that do not permit medical follow-up and compliance with the study protocol.

For Patients Receiving Pembrolizumab on Study (Phase I Backfill Cohort -f, -g, -h, and -i of Group 1, Group 2, Group 3, or Group 4; All Patients in Phase II Group B or Group E)
17. History of severe hypersensitivity reaction to pembrolizumab.
18. Any contraindication to receiving pembrolizumab per package insert or SmPC
19. Allogeneic tissue/solid organ transplant.
20. History of (non-infectious) pneumonitis that required steroids or current pneumonitis.

4. Treatment Regimens

4.1 Phase I Regimens

Group 1 (Intravenous Administration of Construct 1): From Cycles 1 to 4, Construct 1 is administered every 3 weeks. Starting at Cycle 5 and onwards, Construct 1 is administered every 6 weeks. This schedule is referred to as “q3w, q6w.” For Cycles 1 to 4, a treatment cycle is defined as a period of 21 days. Construct 1 doses is administered IV on Day 1 (3 days) of each cycle. For Cycle 5 and subsequent cycles, a treatment cycle is defined as a period of 42 days. Construct 1 doses is administered IV on Day 1 (±7 days) of each cycle.

Group 2 (Single Intratumoral Administration of Construct 1 Followed by Intravenous Administration of Construct 1): From Cycles 1 to 4, Construct 1 is administered every 3 weeks. Starting at Cycle 5 and onwards, Construct 1 is administered every 6 weeks. This schedule is referred to as “q3w, q6w.” For Cycles 1 to 4, a treatment cycle is defined as a period of 21 days. Treatment begins with a single IT administration of Construct 1 on Day 1 of Cycle 1. Subsequent Construct 1 doses are administered IV on Day 1 (±3 days) of Cycle 2 and thereafter. For Cycle 5 and subsequent cycles, a treatment cycle is defined as a period of 42 days. Construct 1 doses are administered IV on Day 1 (±7 days) of each cycle.

IT administration may be performed by image-guided procedures such as interventional radiology. Methodology of IT administration is as per institutional standard. If delivery of the total volume by direct IT administration is not technically feasible, the remaining Construct 1 volume should be delivered peritumorally and/or local administration (Section 5).

Patients have one lesion selected for biopsy and IT administration of Construct 1. The lesion selected is not assessed for tumor response following RECIST and iRECIST (e.g., lesion can be followed individually for change in diameter but should not be included in the overall sum of diameters for RECIST assessment).

Group 3 (Intravenous Administration of Construct 1 and Construct 2): For Cycles 1 and 2, a treatment cycle is defined as a period of 42 days. Patients are administered first Construct 2, then followed by Construct 1, alternating treatment every 3 weeks (21 days), and have a window of ±3 days as follows: Construct 2 is administered IV on Day 1 of Cycles 1 and 2. Construct 1 is administered IV on Day 22 of Cycles 1 and 2. For Cycle 3 and subsequent cycles, a treatment cycle is defined as a period of 84 days. Cycle 3, Day 1 starts following the completion of Cycle 2, Day 42. Construct 2 and Construct 1 doses are administered sequentially, alternating every 6 weeks (42 days), with a window of ±7 days, as follows: Construct 2 is administered IV on Day 1 of Cycle 3 and subsequent cycles. Construct 1 is administered IV on Day 43 of Cycle 3 and subsequent cycles. This schedule is referred to as “q3w, q6w.”

Group 4 (Single IT Administration of Construct 1, Followed by Alternating Intravenous Administration of Construct 2 and Construct 1): Treatment begins with an initial IT administration of Construct 1 on Cycle 0, Day 1. 21 days (3 weeks) later, treatment continues with IV administration of Construct 2 on Day 1 of Cycle 1 and IV administration of Construct 1 on Day 22. For Cycles 1 and 2, a treatment cycle is defined as a period of 42 days. Construct 2 and Construct 1 doses are administered sequentially, alternating every 3 weeks (21 days), with a window of ±3 days, as follows: Construct 2 is administered IV on Day 1 of Cycles 1 and 2. Construct 1 is administered IV on Day 22 of Cycles 1 and 2. For Cycle 3 and subsequent cycles, a treatment cycle is defined as a period of 84 days. Day 1 of Cycle 3 starts following the completion of Day 42 of Cycle 2. Construct 2 and Construct 1 doses are administered sequentially, alternating every 6 weeks (42 days), with a window of ±7 days, as follows: Construct 2 is administered IV on Day 1 of Cycle 3 and subsequent cycles. Construct 1 is administered IV on Day 43 of Cycle 3 and subsequent cycles. This schedule is referred to as “q3w, q6w.” The IT administration is similar to the one described in group 2 above.

Group 5 (Three Doses of Intravenous Administration of Construct 1): Treatment Group 5 explores administering 3 doses of Construct 1 monotherapy. Patients receive IV administration of Construct 1 every 3 weeks and stop after the third dose is received. This treatment plan is referred to as “3-dose Construct 1.” A treatment cycle is defined as a period of 21 days (3 weeks). The 3 doses of Construct 1 are given 3 weeks apart on Day 1 (±3 days) of Cycles 1, 2, and 3. Tumor scan for efficacy assessment is performed every 42 days (6 weeks) starting from the first dose of Construct 1 administered. Tumor response is measured using RECIST until disease progression. Upon disease progression per RECIST, iRECIST is used to assess tumor response.

Upon radiological progression as defined by RECIST or iRECIST and after the patient has received the full 3-dose regimen, patient may receive another 3 doses of Construct 1 administered 3 weeks apart. Patients with disease progression during the 3-dose regimen are not eligible to receive the additional 3 doses. The efficacy assessment is re-baselined to RECIST. Tumor scan(s) continue every 42 days (6 weeks). Upon disease progression per RECIST, iRECIST is used to assess tumor response. Following disease progression per iRECIST, the patient proceeds to study EOT visit and complete the required assessments.

Group 6 (Three Doses of Intravenous Administration of Construct 2 and Construct 1): Treatment Group 6 explores administering 3 doses of Construct 2/Construct 1 alternating 2-vector therapy. Patients receive 3 doses of Construct 2 and Construct 1 each, of which they receive 6 doses in total. A treatment cycle is defined as a period of 42 days. In each cycle, Construct 2 is administered first, followed by Construct 1, in an alternating manner. Each dose is given 3 weeks apart, with a window of ±3 days as follows: Construct 2 is administered IV on Day 1 of Cycle 1, 2, and 3. Construct 1 is administered IV on Day 22 of Cycle 1, 2, and 3. This treatment plan is referred to as “3-dose Construct 1 & Construct 2.”

Tumor scan for efficacy assessment is every 42 days starting from the first dose of Construct 2 administered. Tumor response is measured using RECIST until disease progression. Upon radiological progression defined by RECIST or iRECIST and after the patient has received the full 3-dose regimen, another “3-dose Construct 2 & Construct 1” treatment may be given. Patients with disease progression during the “3-dose Construct 2 & Construct 1” regimen would not be eligible to receive the additional 3 doses. The efficacy assessment is re-baselined to RECIST. Tumor scan(s) continue every 42 days (6 weeks). Upon disease progression per RECIST, iRECIST is used to assess tumor response. Following disease progression per iRECIST, the patient proceeds to study EOT visit and complete the required assessments.

4.2 Phase II Dose Expansion Study Treatment Schedule

The dosing schedule for Phase II Dose Expansion is similarly selected based on the review of the available data from the safety, efficacy, and/or biomarker results of the Dose Escalation Treatment Groups. The study treatment dosing schedule for Dose Expansion could be one of the following:
Construct 1 and/or 2 study treatment administered per the “q3w, q6w” schedule.
Construct 1 and/or 2 study treatment administered every 2 weeks. This schedule is referred to as “q2w”.
Construct 1 and/or 2 study treatment administered every 4 weeks from Cycles 1 to 4 and every 8 weeks starting in Cycles 5 and after. This schedule is referred to as “q4w, q8w”.
Three-dose regimen of Construct 1 and/or 2 treatment, with each dose given 3 weeks apart. This treatment plan is referred to as “3-dose Construct 1 and/or 2 treatment.”

Group A (Intravenous Administration of Construct 1): Phase II Dose Expansion Group A of Construct 1 monotherapy can commence upon completion of Phase I Dose Escalation Group 1 (with determination of the RP2D of Construct 1 when administered IV). For Cycles 1 to 4, a treatment cycle is defined as a period of 21 days: Construct 1 doses are administered IV on Day 1 (±3 days) of each cycle. For Cycle 5 and subsequent cycles, a treatment cycle is defined as a period of 42 days: Construct 1 doses are administered IV on Day 1 (±7 days) of each cycle.

Group B (Intravenous Administration of Construct 1 and Pembrolizumab): Phase II Dose Expansion Group B of Construct 1 monotherapy and pembrolizumab can commence upon completion of Phase I Dose Escalation Group 1 (with determination of the RP2D of Construct 1 when administered IV). The patient are receiving Construct 1 monotherapy and pembrolizumab. For Cycles 1 to 4, a treatment cycle is defined as a period of 21 days. Construct 1 doses are administered IV only on Day 1 (±3 days) of each cycle. For Cycle 5 and subsequent cycles, a treatment cycle is defined as a period of 42 days. Construct 1 is administered IV on Day 1 (±7 days) of each cycle. Pembrolizumab is administered on a q3w or q6w schedule, overlapping with study visits.

Group C (Single IT Administration of Construct 1 Followed by Intravenous Administration of Construct 1): Phase II Dose Expansion Group C of Construct 1 monotherapy can begin upon completion of Phase I Dose Escalation Group 2 (with determination of the RP2D of Construct 1 IV and IT). For Cycles 1 to 4, a treatment cycle is defined as a period of 21 days. Treatment begins with a single IT administration of Construct 1 on Day 1 of Cycle 1. Subsequent Construct 1 doses are administered IV on Day 1 (±3 days) of each cycle. For Cycle 5 and subsequent cycles, a treatment cycle is defined as a period of 42 days. Construct 1 doses are administered IV on Day 1 (±7 days) of each cycle.

IT administration may be performed by image-guided procedures such as interventional radiology. Methodology of IT administration is as per institutional standard. Ideally, all the volume should be delivered via direct IT administration. If delivery of the total volume by direct IT administration is not technically feasible, the remaining Construct 1 volume should be delivered peritumorally and/or local administration (see Section 5).

Patients have one lesion selected for biopsy and IT administration of Construct 1. The lesion selected is not assessed for tumor response following RECIST and iRECIST (e.g., lesion can be followed individually for change in diameter but should not be included in the overall sum of diameters for RECIST assessment). All other lesions do not receive Construct 1 or be biopsied.

Group D (Sequential Alternating Intravenous Administrations of Construct 2 and Construct 1): Phase II Dose Expansion Group D of Construct 2/Construct 1 alternating 2-vector therapy can begin upon completion of the Phase I Dose Escalation Group 3 (with determination of the RP2D of Construct 2 when administered IV with Construct 1 in a sequential alternating schedule). For Cycles 1 and 2, a treatment cycle is defined as a period of 42 days. Treatment begins with IV administration of Construct 2 on Day 1 of Cycle 1, followed by Construct 1 alternating every 3 weeks (21 days) as specified below. Construct 2 and Construct 1 dose administrations have a window of ±3 days. Construct 2 is administered IV on Day 1 of Cycles 1 and 2. Construct 1 is administered IV on Day 22 of Cycles 1 and 2. For Cycle 3 and subsequent cycles, a treatment cycle is defined as a period of 84 days. Day 1 of Cycle 3 starts following the completion of Day 42 of Cycle 2. Construct 2 and Construct 1 dose administrations in Cycle 3 and subsequent cycles have a window of ±7 days. Construct 2 and Construct 1 doses alternate every 6 weeks (42 days) as follows: Construct 2 is administered IV on Day 1 of Cycle 3 and subsequent cycles. Construct 1 is administered IV on Day 43 of Cycle 3 and subsequent cycles.

Group E (Sequential Alternating Intravenous Administration of Construct 2 and Construct 1, and Pembrolizumab): Phase II Dose Expansion Group E Construct 2/Construct 1 alternating 2-vector therapy and pembrolizumab can begin upon completion of the Phase I Dose Escalation Group 3 (with determination of the RP2D of Construct 2 when administered IV with Construct 1 in a sequential alternating schedule). For Cycles 1 to 2, a treatment cycle is defined as a period of 42 days. Treatment begins with IV administration of Construct 2 on Day 1 of Cycle 1. Construct 2 and Construct 1 dose administrations in Cycles 1 and 2 have a window of 3 days. Patients are administered the first two doses of Construct 2 and Construct 1 alternating every 3 weeks (21 days) as follows: Construct 2 is administered IV on Day 1 of Cycles 1 and 2. Construct 1 is administered IV on Day 22 of Cycles 1 and 2. For Cycle 3 and subsequent cycles, a treatment cycle is defined as a period of 84 days. Day 1 of Cycle 3 starts following the completion of Day 42 of Cycle 2. Construct 2 and Construct 1 dose administrations in Cycle 3 and subsequent cycles have a window of ±7 days. Construct 2 and Construct 1 doses alternate every 6 weeks (42 days) as follows: Construct 2 is administered IV on Day 1 of Cycle 3 and subsequent cycles. Construct 1 is administered IV on Day 43 of Cycle 3 and subsequent cycles. Pembrolizumab is administered on a q3w or q6w schedule, overlapping with study visits.

Group F (Intratumoral Administration of Construct 1 Followed by Sequential Alternating Intravenous Administrations of Construct 2 and Construct 1):

Phase II Dose Expansion Group F can begin upon completion of the Phase I Dose Escalation Group 4 (with determination of the RP2D of Construct 1 when administered IT and followed by a sequential alternating schedule of the RP2D of Construct 2 and Construct 1 when administered IV).

Treatment begins with an initial IT administration of Construct 1 on Day 1 of Cycle 0. IT administration may be guided by image guided procedures such as interventional radiology. 21 days later, treatment continues with IV administration of Construct 2 on Day 1 of Cycle 1 and IV administration of Construct 1 on Day 22. Ideally, all the volume should be delivered via direct IT administration. If delivery of the total volume by direct IT administration is not technically feasible, the remaining Construct 1 volume should be delivered peritumorally and/or local administration (see Section 5).

For Cycles 1 and 2, a treatment cycle is defined as a period of 42 days. Construct 2 and Construct 1 dose administrations in Cycle 2 have a window of ±3 days. Patients are administered Construct 2 and Construct 1, alternating every 3 weeks (21 days) as follows: Construct 2 is administered IV on Day 1 of Cycles 1 and 2. Construct 1 is administered IV on Day 22 of Cycles 1 and 2. For Cycle 3 and subsequent cycles, a treatment cycle is defined as a period of 84 days. Day 1 of Cycle 3 starts following the completion of Day 42 of Cycle 2. Construct 2 and Construct 1 dose administrations in Cycle 3 and subsequent cycles have a window of ±7 days. Construct 2 and Construct 1 doses alternate every 6 weeks (42 days) as follows: Construct 2 is administered IV on Day 1 of Cycle 3 and subsequent cycles. Construct 1 is administered IV on Day 43 of Cycle 3 and subsequent cycles. IT administration is similar to the one in group C above.

4.3 Addition of Pembrolizumab Upon Disease Progression

For patients who are enrolled in treatment Groups 1, 2, 3, or 4 only and have subsequently progressed radiologically as defined by iRECIST, pembrolizumab can be added to the Construct 1 and/or 2 treatment. Eligibility to receive pembrolizumab should be assessed using inclusion and exclusion criteria pertaining to the pembrolizumab cohorts (Refer to Section 3). Dosing of pembrolizumab should overlap with study visits and follow the q3w or q6w schedule depending on the Construct 1 monotherapy or Construct 2/Construct 1 alternating 2-vector therapy assigned treatment schedule.

The efficacy assessment is re-baselined using RECIST v1.1 when pembrolizumab is added to the Construct 1 monotherapy or Construct 2/Construct 1 alternating 2-vector therapy. Upon disease progression per RECIST, iRECIST is used to assess tumor response.

5. Treatment Administration

The total volume of Construct 1 for IT administration depends on the provisional Construct 1 dose prescribed (see Table 3). One lesion and/or site of disease is selected for Construct 1 IT administration. This should be the same lesion/site of disease that was selected for biopsy pre- and post Construct 1 IT administration. Ideally, all the volume should be delivered via direct IT administration. If delivery of the total volume by direct IT administration is not technically feasible, the remaining Construct 1 volume should be delivered peritumorally and/or local administration for the primary purpose of treating one specific lesion or site of disease. To ensure the entire volume of Construct 1 dose prescribed is administered, the following types of administrations are allowed:
Direct IT administration (tumor leakage, if it occurs is acceptable);
Peritumoral administration (if direct IT injection is technically difficult);
Local administration in the vicinity of the tumor (if IT and peritumoral injection is technically difficult).

The delivery of the total IT volume may occur via one injection or via more than one injection, or via one injection and multiple re-positioning of the needle without withdrawal, or a combination.

Pembrolizumab should be administered per institutional guidelines or per standard of care, such as the appropriate KEYTRUDA® SmPC or Package Insert.

6. Dosing

6.1 Dose Levels Explored for Construct 1

Table 3 below describes the Construct 1 starting dose and the dose levels that may be evaluated during Phase I Dose Escalation (Groups 1 and 2). For Phase I Dose Escalation Group 1 (Construct 1 IV only), the starting dose of Construct 1 starts at 5×10⁵ RCV FFU. The subsequent dose of Construct 1 is increased to the next sequential dose level as listed in Table 3. For Phase I Dose Escalation Group 2 (Construct 1 IT-IV), the starting dose for IT administration of Construct 1 starts at 5×10⁵ RCV FFU. The starting dose for IV administration also starts at 5×10⁵ RCV FFU. The subsequent doses of Construct 1 for IT and IV administration are the same and both are increased to the next sequential dose level as listed in Table 3.

For Phase I Dose Escalation Group 5 (3-dose Construct 1 regimen), the dose can start one log order up from highest dose level declared safe in Group 1. As an example, at the time when Group 5 enrolls patients and the highest Construct 1 dose declared safe in Group 1 is 5×10⁶ RCV FFU, the Construct 1 dose level Group 5 can explore is 5×10⁷ RCV FFU. The subsequent dose level of Construct 1 is increased to the next sequential dose level as listed in Table 3.

TABLE 3
Provisional Construct 1 Dose Escalation

| Level/Cohort | Construct 1 Dose |
|---|---|
| −1 | 5 × 10⁴ RCV FFU |
| 1 (starting dose) | 5 × 10⁵ RCV FFU |
| 2 | 5 × 10⁶ RCV FFU |
| 3 | 5 × 10⁷ RCV FFU |
| 4 | 1 × 10⁸ RCV FFU or |
| | 5 × 10⁸ RCV FFU |

FFU = focus-forming units, RCV = replication-competent virus.

6.2 Provisional Dose Levels Explored for Construct 2/Construct 1 Alternating 2-Vector Therapy

The proposed human starting dose of Construct 2 is 1×10⁶ RCV FFU. The proposed human starting dose of Construct 1 is the highest dose level declared safe in Group 1 or Group 2. If RP2D is declared for Construct 1 from monotherapy, then Construct 1 dose level in Construct 2/Construct 1 alternating 2-vector therapy remains at the RP2D, while Construct 2 provisional levels are explored. Table 4 describes the starting dose for the Construct 2/Construct 1 alternating 2-vector therapy and the dose levels that may be evaluated during Phase I Dose Escalation (Groups 3 and 4).

For Phase I Dose Escalation Group 6 (3-dose Construct 2 & Construct 1 IV), the dose of Construct 2 and Construct 1 can start one log order up from highest dose level declared safe in Group 3. As an example, at the time when Group 6 enrolls patients and the highest Construct 2 and Construct 1 alternating 2-vector treatment dose declared safe in Group 3 is 5×10⁶ RCV FFU for Construct 1 and 1×10⁶ RCV FFU for Construct 2, the dosages for Group 6 can be at 5×10⁷ RCV FFU for Construct 1 and 1×10⁷ RCV FFU for Construct 2. The subsequent dose levels of Construct 2 and Construct 1 are increased to the next sequential dose level as listed in Table 4.

TABLE 4
Provisional Construct 2 and Construct 1 Doses

| Cohort | Construct 2 Dose | Construct 1 Dose^{a} |
|---|---|---|
| −1 | 1 × 10⁵ RCV FFU | One log order down from |
| | | starting dose |
| 1 (starting | 1 × 10⁶ RCV FFU | RP2D or highest safe dose^{b} |
| dose) | | |
| 2 | 1 × 10⁷ RCV FFU | Up to one log order up from |
| | | starting dose |
| 3 | 1 × 10⁸ RCV FFU | Up to two log orders up from |
| | | starting dose |
| 4 | 1 × 10⁹ RCV FFU | Up to three log orders up from |
| | | starting dose |

FFU = focus-forming units, RCV = replication-competent virus, RP2D = recommended Phase II dose.
^{a}The Construct 1 or Construct 2 dose explored in the cohort may be a dose level lower than indicated in the provisional dose table.
^{b}The dose of Construct 1 in Cohort 1 is the RP2D determined in Groups 1 and 2, or the highest dose determined to be safe if the RP2D has not been reached.

6.3 Dosing for Pembrolizumab Pembrolizumab is administered on a 200 mg once every 3 weeks or 400 mg once every 6 weeks schedule for Groups B and E of Phase II. 7. Efficacy Assessment

Efficacy is assessed utilizing CT or MRI scans of the chest/abdomen/pelvis and all suspected anatomic regions involved with the disease and are performed to assess tumor response. For chest scans, CT modality is mandatory. Ultrasound should not be used to measure sites of disease. If a CT/MRI scan is scheduled on the same day as study treatment administration, the CT/NRI should be performed prior to dosing.

For patients who have subsequently progressed radiologically as defined by iRECIST and for whom pembrolizumab is introduced while continuing Construct 1 monotherapy or Construct 2/Construct 1 alternating 2-vector therapy (Section 5.3.5), the efficacy assessment is re-baselined to RECIST. Upon disease progression per RECIST, iRECIST is used to assess tumor response.

Any Complete response (CR) or Partial response (PR) should be confirmed, preferably at the scheduled interval, but no sooner than 4 weeks after the initial documentation of CR or PR. Confirmation of CR or PR can be confirmed at the next evaluable tumor assessment after the initial documentation of CR or PR.

TABLE 5
Tumor Response Assessment

| Efficacy | | |
|---|---|---|
| Assessment | Evaluation | Efficacy Endpoint |
| Primary | RECIST | ORR (Phase II Dose Expansion only) |
| Secondary | iRECIST | ORR (Phase II Dose Expansion only) |
| Secondary | RECIST and | ORR (Phase I Dose Escalation only) |
| | iRECIST | Duration of response |
| | | Disease control rate |
| | | PFS |

iRECIST = immune Response Evaluation Criteria in Solid Tumors,
ORR = objective response rate,
PFS = progression-free survival,
RECIST = Response Evaluation Criteria in Solid Tumors

8. Safety Assessments

Certain safety measurements that are well known in the art are performed, such as physical examination, vital signs, height, weight, electrocardiograms, and clinical laboratory parameters.

In addition, samples from saliva, feces (e.g., fecal swab), blood, and urine are collected for viral shedding analysis. Viral shedding is analyzed by quantitative reverse transcription PCR to quantify the copies of nucleoprotein RNA, and may be coupled with infectivity assay to characterize the shed material to confirm absence of infectious virus. For viral shedding collection, samples collected should not be from areas potentially containing viable cancer cells.

9. Biomarkers

9.1 Biomarker Assessments in Blood

To address the exploratory objective of identifying possible Construct 1, Construct 2, and pembrolizumab PD markers, a few exploratory candidate biomarkers are evaluated (see Table 6).

During the study, blood samples (including serum and plasma) are collected for phenotypic, genomic, proteomic, and transcriptional analyses. Blood samples are collected for phenotypic characterization of lymphocyte subsets. Messenger RNA expression profiling in blood is performed to evaluate gene signatures associated with clinical response and/or resistance. Plasma is collected for circulating tumor DNA assessment. Neutralizing and binding antibodies against study treatment (essentially, E7E6 fusion protein), and pro inflammatory, Th1/Th2 cytokines such as IL 1, IL 12, and IL-18, TNF, and IFN-γ are assessed in serum.

TABLE 6
Summary of Biomarker Sample Collection and Analyses

| Sample Type | Biomarker Analyses |
|---|---|
| Blood/Plasma | Lymphocyte subsets (TruCount) |
| | ctDNA |
| | Transcriptional analysis (RNA-seq) |
| Serum | Cytokines |
| | Neutralizing antibodies |
| | bAb |
| Tumor tissue | IHC TIL |
| | Transcriptome analysis (RNA-seq) |
| | WES analysis |

bAb = anti-drug antibodies, ctDNA = circulating tumor deoxyribonucleic acid, GEP = gene expression profile, IHC = immunohistochemistry, MHC = major histocompatibility complex, RNA = ribonucleic acid, TCR = T cell receptor, TIL = tumor-infiltrating lymphocyte, WES = whole exome sequencing.

9.2 Biomarker Assessments in Tumor Tissues

Tumor tissue samples are collected with the purpose of investigating effects of Construct 1 monotherapy and Construct 2/Construct 1 alternating 2-vector therapy on molecular signaling and tumor cell responses, identifying biomarkers that may be predictive of efficacy and response.

Tumor tissue are obtained by image-guided biopsy, such as interventional radiology. All patients submit tissue from either a core or excisional biopsy (fine needle aspirate not accepted) to central laboratory for biomarker assessment.

The application of new technologies, such as next generation sequencing, provides the opportunity to assess at the genetic level in the tumor. Next generation biomarkers, such as gene expression profile signatures by RNA sequencing, microsatellite instability, tumor mutational burden, and human leukocyte antigen loss of heterozygosity are examined in tumor tissues obtained from patients to understand the potential biomarker of clinical response and/or resistance.

In addition, tumor material is used to quantify the levels of tumor infiltrated lymphocytes by immunohistochemistry staining. The analysis provide assessment of tumor infiltration of immune cells and particularly CD8⁺ T cells in the tumor.

9.2.1 Tumor Tissue Sample Collection

Tumor samples are collected at Screening or on the day of their first study drug administration and post dose. Fresh tumor biopsy should be provided, if accessible. If tumor biopsy and CT/MRI scan are performed on the same visit, the CT/MRI scan should be performed first, followed by the tumor biopsy.

For patients receiving Construct 1 IT as the first dose (Day 1 of Cycle 1 pre-dose for Construct 1 monotherapy or Day 1 of Cycle 0 pre-dose for Construct 2/Construct 1 alternating 2-vector therapy):
One lesion is selected for biopsy prior to Construct 1 IT administration.
The same lesion is also administered with the Construct 1 IT administration, unless agreed otherwise between the Sponsor and the Investigator.

For patients recruited to enroll in the backfill cohorts as specified in FIG. 16, paired fresh tumor biopsies are required prior to first study drug administration and post dose before the first efficacy assessment CT/MRI scan for participation in the backfill cohorts.

Tissue samples must be newly obtained from either a core or excision biopsy (fine needle aspirate not accepted) for biomarker assessment. Submission of the tumor block with largest tumor focus (minimum of two cores) or highest tumor cellularity is required. Tumor blocks of resection/excision specimens are preferred over slides.

9.3 Other Exploratory Biomarker Assessments

In addition to the biomarkers specified in Sections 9.1 and 9.2, exploratory biomarker research may be conducted on any tumor tissue, serum/plasma, and peripheral mononuclear cells (PBMC) samples collected during the example. These additional investigations could extend the search for other potentially relevant biomarkers for the Construct 1 and/or Construct 2 effect, and/or safety. The additional exploratory biomarkers include, tetramer sorted antigen specific T cell profiling, T cell receptor sequencing and chromatin changes on antigen specific T cells.

10. Immunogenicity

Immunogenicity testing is done in all patients to monitor patients' CD8⁺ T cells functionality and antigen recognition by measuring IFN-γ, TNF-α, IL-2, CD107a via intracellular staining and secreted IFN-γ specific cells in peripheral blood mononuclear cells as an antigen specific immune response against Construct 1 and/or Construct 2, with and without pembrolizumab (see Table 7).

TABLE 7
Summary of Immunogenicity Analysis

| Sample | |
|---|---|
| Type | Immunogenicity Analysis |
| Blood | ICS panel CD4 and CD8: IFN-γ, TNF-α, |
| | IL-2, CD107a and CD154 |
| (PBMCs) | ELISpot assay measuring secreted IFN-γ |
| | using E7E6 based peptides, |
| | LCMV, and PICV NP peptides. |

CD4 = cluster of differentiation 4, CD8 = cluster of differentiation 8, E7E6 = antigenic E7 and E6 fusion protein from human papillomavirus 16, ELISpot = enzyme-linked immune absorbent spot, ICS = intracellular cytokine staining, IFN-γ = interferon-gamma, IL-2 = interleukin-2, LMCV = lymphocytic choriomeningitis virus, NP = nucleoprotein, PBMC = peripheral blood mononuclear cell; PICV = Pichinde Virus, TNF-α = tumor necrosis factor alpha.

11. Exploratory Imaging Sub-Study

The aim of this exploratory immune imaging objective is to capture the distribution and influx of CD8⁺ cells into tumor tissues upon treatment with Construct 1 monotherapy or Construct 2/Construct 1 alternating 2-vector therapy. Specifically, the distribution of CD8⁺ cells by assessing whole body PET/CT images using CD8 PET Tracer is measured to evaluate changes before and after treatment with Construct 1 monotherapy or Construct 2/Construct 1 alternating 2-vector therapy. Clinical outcome is correlated through quantification of CD8 PET Tracer signal. Furthermore, evaluating the change in CD8 PET Tracer signal before and after treatment is used to predict treatment efficacy, and true radiological progression and pseudo-progression during the early phase of Construct 1 monotherapy and Construct 2/Construct 1 alternating 2-vector therapy are also distinguished.

During the Phase I Dose Escalation portion of the example, a sub-study is carried out to include exploratory immune imaging with positron emission tomography (PET)/CT scan to assess ⁸⁹Zr-Df-IAB22M2C (CD8 PET Tracer, an anti-CD8 minibody (IAB22M2C), conjugated with deferoxamine (Df) and radiolabeled with Zirconium-89 (⁸⁹Zr-Df-IAB22M2C)) in patients with HNSCC receiving Construct 1 monotherapy or Construct 2/Construct 1 alternating 2-vector therapy. A dose of 1.0 (±20%) mCi of CD8 PET Tracer 1.5 mg of API is administered IV over 5-10 minutes. CD8 PET Tracer uptake in tumors is determined by standardized uptake value (SUV)-based quantitative measures (SUVmax, SUVpeak, SUVmean, CD8 tumor volume). Volume of tumor tissues with increased CD8 uptake with SUV >20% SUVmax is quantified.

Two backfill cohorts of HPV 16⁺ confirmed cancer patients for Phase I Dose Escalation treatment Group 1 and Group 3 (see Section 2.1 in Example III), respectively, are enrolled for the CD8 PET Tracer imaging study. For patients from group 1, CD8 PET Tracer as an IV infusion is received within 2 weeks (14 days) prior to the first dose of Construct 1 IV administration (Baseline imaging), and 8 to 12 days after the second dose of Construct 1 administration (post-treatment imaging). PET/CT scans (PET Baseline and PET Post-Treatment) are obtained at 24±3 hours after each infusion of CD8 PET Tracer. For patients from group 2, CD8 PET Tracer as an IV infusion is received within 2 weeks (14 days) prior to the first dose of Construct 2 IV administration (Baseline imaging), and 8 to 12 days after the first dose of Construct 1 administration (post-treatment imaging). PET/CT scans (PET Baseline and PET Post-Treatment) are obtained at 24±3 hours after each infusion of CD8 PET Tracer.

### Example IV

Effective Arenavirus-Based Cancer Immunotherapy in Patients with HPV16⁺ Cancers

This example illustrates the preliminary data for the dose escalation portion of Example III.

Total 38 patients were enrolled, out of which 32 patients were diagnosed with HNSCC, and 6 patients were diagnosed with non-HNSCC HPV16⁺ cancers (see Table 8)

TABLE 8
Baseline Characteristics of Patients Enrolled

| Cohorts | Total | | HNSCC | | Non-HNSCC | |
|---|---|---|---|---|---|---|
| Number of HPV16+ | 38 | | 32 | | 6 | |
| patients | | | | | | |
| Primary Site | | | | | | |
| Oropharynx | 29 | (76.3) | 29 | (90.6) | 0 | (0) |
| Other, n details | 9 | (23.7) | 1 Nasal | | 3 Cervical | |
| | | | 1 Nasopharynx | | 1 Vaginal | |
| | | | 1 Unknown | | 1 Anal | |
| | | | | | 1 Penile | |
| Age, years, median (range) | 62 | (30-86) | 64 | (30-86) | 54 | (49-66) |
| Gender, male | 30 | (78.9) | 29 | (90.6) | 1 | (16.7) |
| Race, White | 34 | (89.5) | 30 | (93.8) | 4 | (80.0) |
| ECOG PS 1 | 23 | (60.5) | 19 | (59.4) | 4 | (66.7) |
| Prior lines of therapy, | 3 | (1-10) | 3 | (1-10) | 3 | (2-3) |
| median (range) | | | | | | |
| Prior CPI use | 31 | (81.6) | 28 | (87.5) | 3 | (50.0) |
| Prior platinum use | 34 | (89.5) | 29 | (90.6) | 5 | (83.3) |
| Prior cetuximab use | 18 | (47.4) | 18 | (56.3) | 0 | (0.0) |
| Distant metastasis at | 30 | (78.9) | 24 | (75.0) | 6 | (100.0) |

baseline

No dose limiting toxicities were observed across any cohort evaluated, and no significant changes were seen across dose levels and regimens. No related serious or related Grade ≥3 events were reported, and no dose reduction, does interruption, discontinuation, or death occurred.

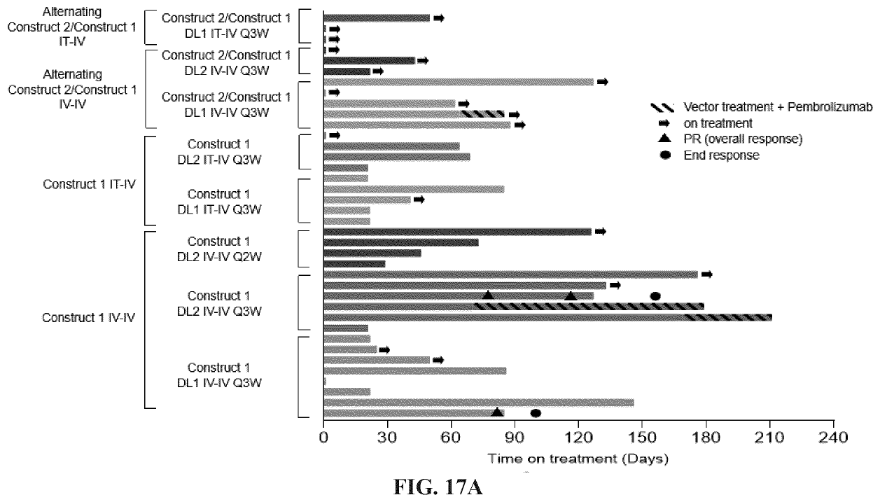

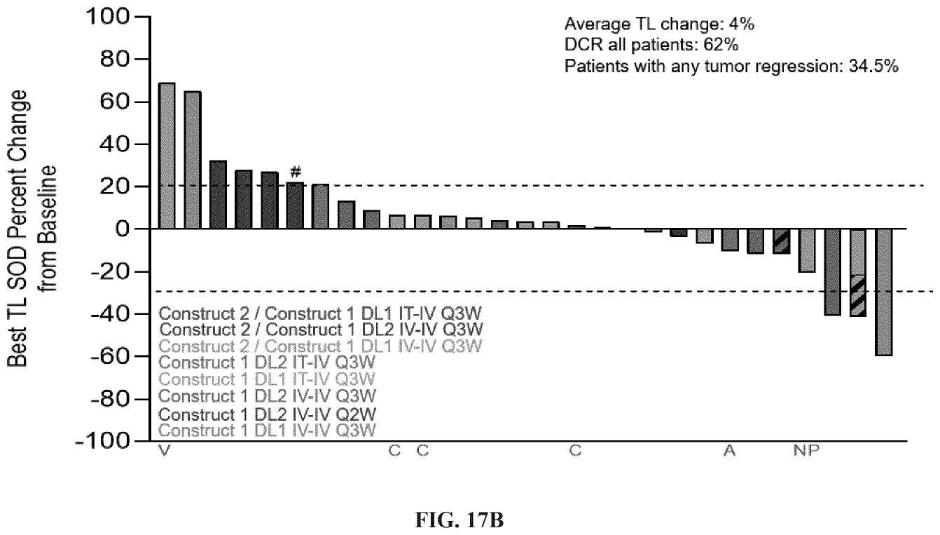

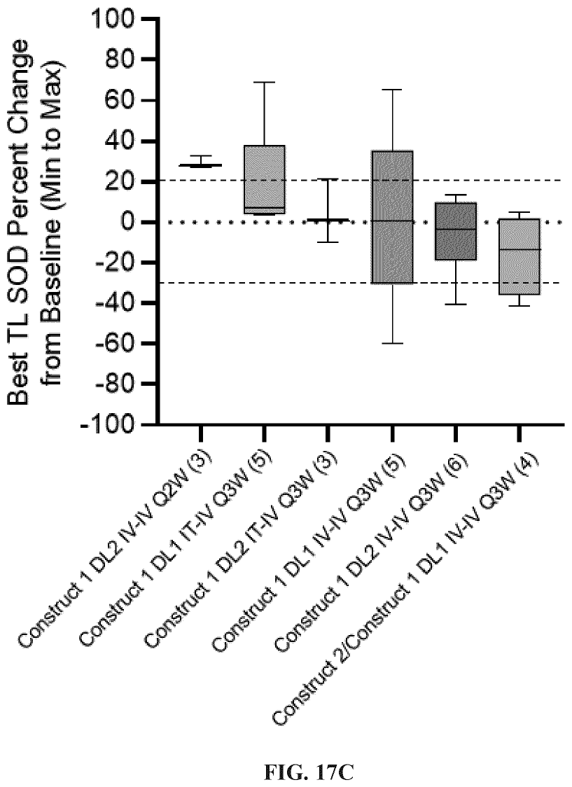

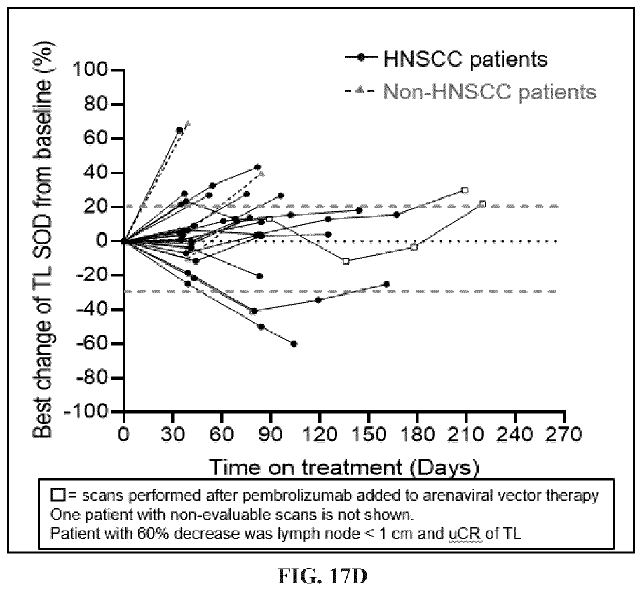

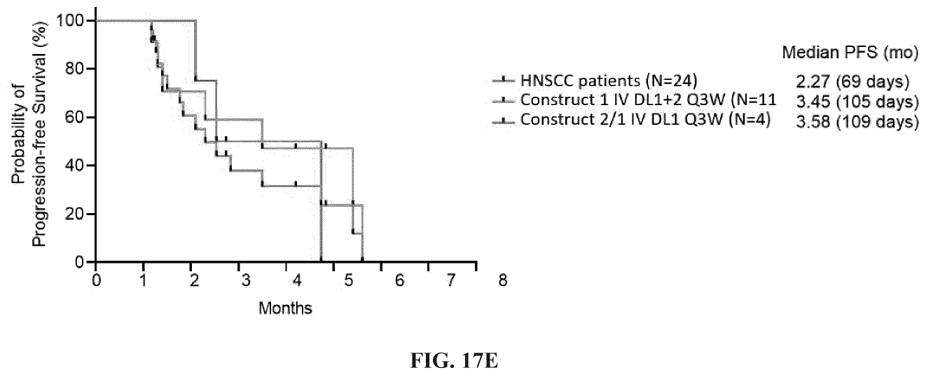

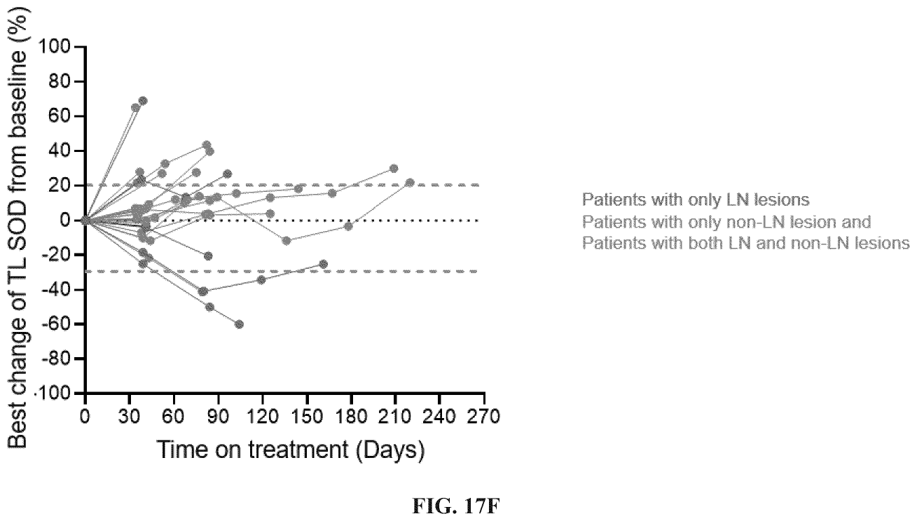

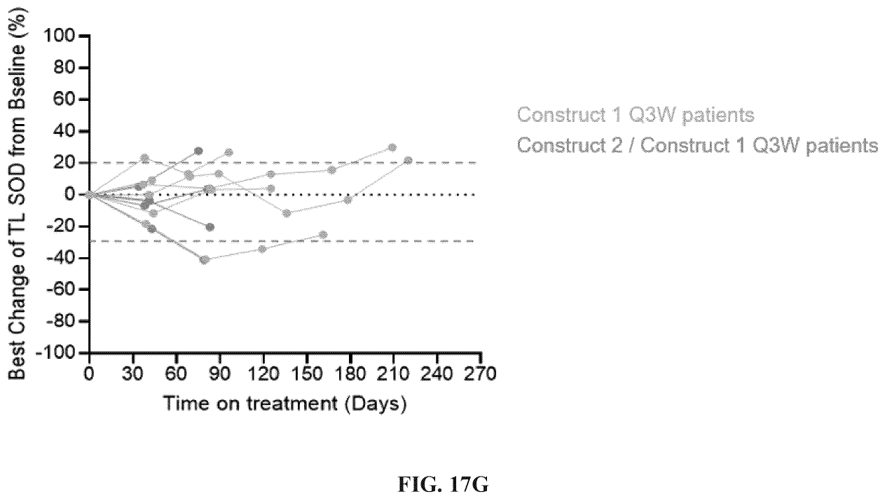

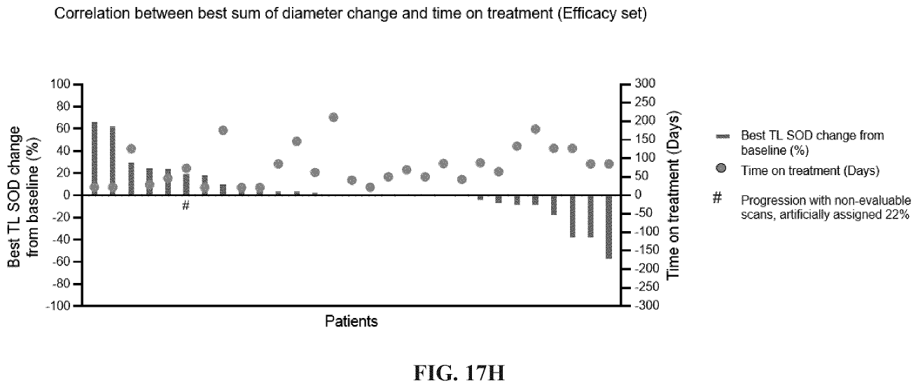

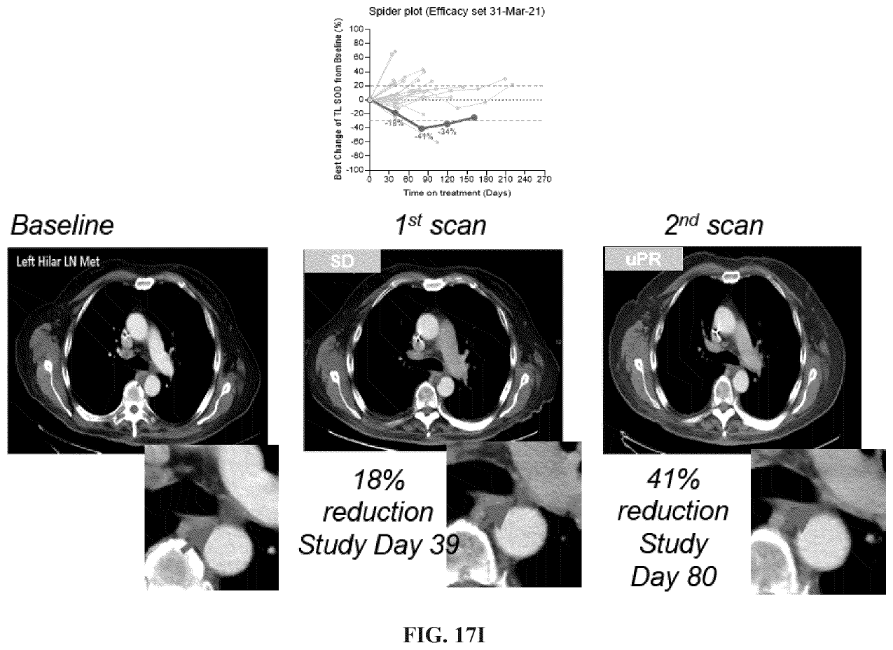

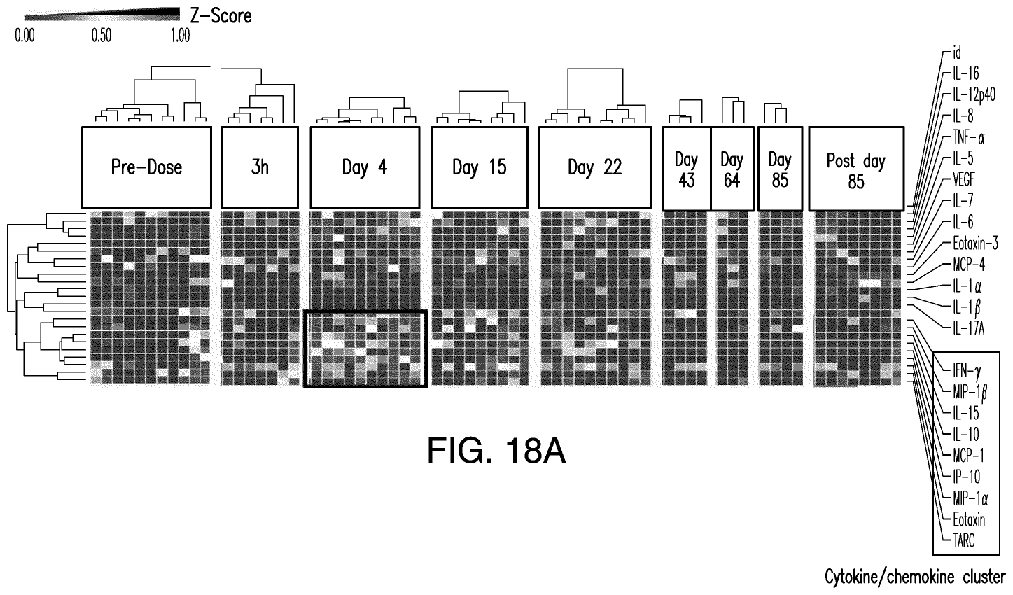

Disease control and prolonged survival were observed among the treated patients. As illustrated in FIG. 17, a significant portion of patients experienced stable disease or tumor shrinkage. The swimmer plot in FIG. 17A shows treatment duration and response to arenaviral vector therapy in individual patients, with time on treatment calculated as last treatment or death minus the first dose date plus one. The waterfall plot in FIG. 17B shows target lesion change from baseline in patients receiving arenaviral vector therapy. Most patients experienced stable disease. Antitumor activity of arenaviral vector therapy was observed in some patients with partial response and tumor reduction. Disease control rate was 62% overall. About 34% of patients experienced tumor regression. To assess differences between different mode of administration, schedules and dose levels, average best target lesions change (SOD) from baseline were analyzed for cohorts >1 patient. Respective results in FIG. 17C demonstrate superiority of intravenous (IV) over intratumoral (IT) administration. Furthermore, a three-weeks interval between vector administrations appeared to be superior to vectors being administered every two weeks. Data further suggest a superior anti-tumor effect in patients treated with the Construct 2/Construct 1 alternating 2-vector therapy as compared to patients receiving the Construct 1 single vector treatment at the same dose. As illustrated in FIG. 17D and Table 9, a significant subset of patients demonstrated stable disease following arenaviral vector therapy. Several endpoints, such as response rate, disease control rate and progression-free survival, of patients receiving arenaviral vector therapy are summarized in Table 9 below. Overall, objective response rate was 6.9% with 2 patients achieving partial response and 16 patients experiencing stable disease. Among those patients with stable disease, 4 maintained this response for at least 16 weeks. Eleven patients had progressive disease and disease control was seen in 18 patients. Median progression-free survival for all patients was 2.27 months. However, head and neck squamous cell carcinoma patients treated with Construct 1, administered intravenously every 3 weeks at dose level 1 or 2, had an objective response rate of 18.2% with 2 patients achieving partial response and 6 patients (54.5%) experiencing stable disease. Median PFS was 3.45 months. In patients receiving Construct 2/Construct 1 alternating 2-vector therapy administered IV every 3 weeks at DL1 for Construct 2 and DL2 for Construct 1, 4 out of 4 patients experienced stable disease, with a median progression-free survival of 3.58 months. FIG. 17E shows the progression-free survival for all HNSCC patients.

TABLE 9
Endpoint Assessments in Treated Patients

| | Construct 1 IV | Construct 1 IV | | | | | |
|---|---|---|---|---|---|---|---|
| | DL1&DL2 | DL2/Construct 2 | | | | | |
| | Q3W HNSCC | IV DL1 Q3W HNSCC | | All HNSCC | | All patients | |
| N, evaluable | 11 | 4 | | 24 | | 29 | |
| (≥1 scan) | | | | | | | |
| ORR, n (%) | 2 (18.2) | 0 | (0.0) | 2 | (8.3) | 2 | (6.9) |
| PR, n (%)^{a} | 2 (18.2) | 0 | (0.0) | 2 | (8.3) | 2 | (6.9) |
| SD, n (%) | 6 (54.5) | 4 | (100.0) | 14 | (58.3) | 16 | (55.2) |
| SD ≥16 wks | 4 (36.4) | 0 | (0.0) | 4 | (16.7) | 4 | (13.8) |
| PD, n (%) | 3 (27.3) | 0 | (0.0) | 8 | (33.3) | 11 | (37.9) |
| DCR, n (%) | 8 (72.7) | 4 | (100.0) | 16 | (66.7) | 18 | (62.1) |

| | | | | |
|---|---|---|---|---|
| PFS^{b}, median (mo) | 4.50 | 3.65 | 2.63 | 2.63 |
| PFS excluding time | 3.45 | 3.58 | 2.27 | 2.27 |
| on pembrolizumab, | | | | |
| median (mo) | | | | |

^{a}PR include 1 confirmed PR and 1 unconfirmed PR;
^{b}In patients who received pembrolizumab with Construct 1 and 2 EDC data was used for some patients due to missing/incorrect data entry on TLF as of the data transfer date.
ORR, objective response rate; PR, partial response; SD, stable disease; PD, progressive disease; DCR, disease control rate; PFS, progression-free survival.
PFS includes time after pembrolizumab had been added prior to RECIST progression.

Interestingly, patients with lymph node lesions as the only target lesions responded better to arenaviral vector therapy than patients having non-lymph node organs as target lesions (see FIG. 17F). Equally interestingly, patients receiving IV administration of Construct 1 DL2 every three weeks (G1DL2Q3W) and IV administration of Construct 2/Construct 1 alternating 2-vector therapy every three weeks (G3 DL1 and DL2 Q3W) showed the best response in target lesions (see FIG. 17G). When comparing best sum of target lesion diameter change and time on treatment, most patients with longer duration on treatment experienced stable disease or tumor reduction. A correlation between the best sum of diameter change and time on treatment is depicted in FIG. 17H. As a case study, in one particular patient with metastatic head and neck squamous cell carcinoma (HNSCC) and primary lesion site of the oropharynx, rapid disease stabilization followed by partial response was observed following intravenous treatment with Construct 1 DL2 monotherapy every three weeks. The respective patient had received the following prior treatments before being treated with Construct 1: (1) Cisplatin+XRT; (2) Carboplatin/paclitaxel/cetuximab; and (3) Pembrolizumab. The CT scans shown in FIG. 17G demonstrate tumor reduction at Days 39 and 80 (see FIG. 17F). The patient continued to receive Construct 1 arenaviral vector therapy for 127 days when disease progression was confirmed.

Taken together, the safety profile of arenavirus-based therapeutic vaccines was specifically acceptable. Both Construct 1 monotherapy and Construct 2/Construct 1 alternating 2-vector therapy were generally well-tolerated in advanced patients with HPV16⁺ tumors. As a single agent without any combination, the therapies demonstrated preliminary antitumor activity in these heavily pre-treated patients with HPV16+ HNSCC (see Table 10 below).

TABLE 10
Comparison between the arenavirus therapy
and 1^{st} and 2^{nd} lines checkpoint inhibitors

| HNSCC | | ORR (%) | PFS (mo) | OS (mo) |
|---|---|---|---|---|
| 1^{st} Line CPI | | 17-23 | 2.2-3.4 | 12-15 |
| 2^{nd} Line CPI | | 14-16 | 2.0-2.3 | 7-9 |
| 3^{rd} Line | All schedules | 8* | 2.27 | NR |
| Arenaviral | Construct 1 | 18.2* | 3.45 | NR |
| vector therapy | IV Q3W | | | |

### Example V

Immunogenicity Induced by Arenavirus-Based Cancer Immunotherapy in Patients with HPV16⁺ Cancers

This example illustrates strong immunogenicity induced by Construct 1 alone as well as Construct 2/Construct 1 alternating 2-vector therapy.

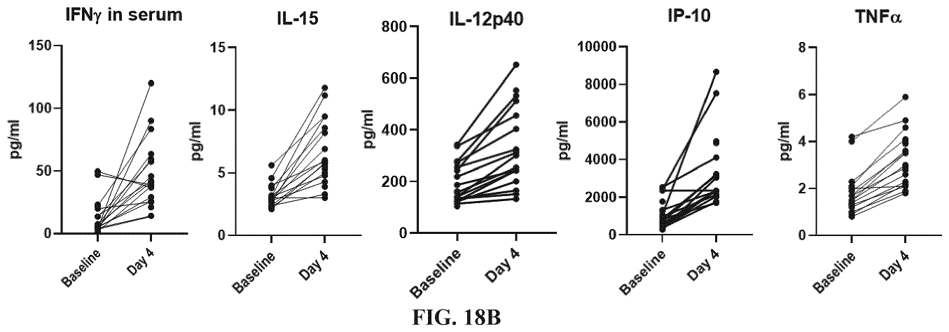

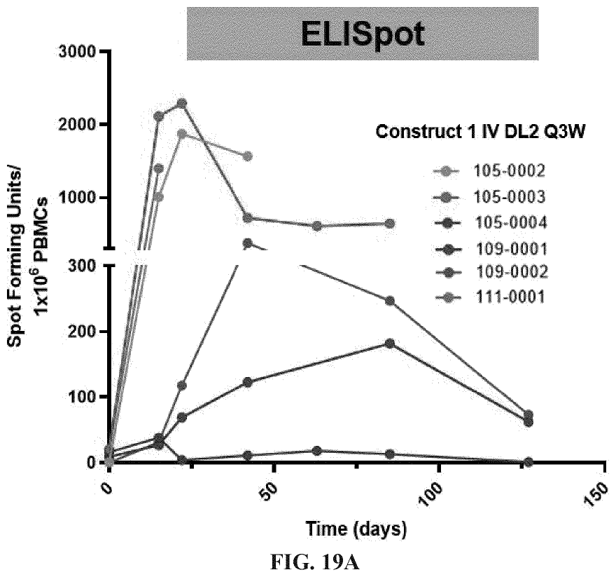

FIG. 18A as an updated FIG. 6A, demonstrates distinct serum cytokine or chemokine signatures after administration of Construct 1. Increased IFN-γ levels are already observed on day 4 after the first treatment in 90% of analyzed patients. Besides IFN-γ other immune stimulatory cytokines and chemokines are also upregulated in treated patients demonstrating early signs of NK and T cell activation. E.g., a single dose of Construct 1 increased levels of IFN-γ, IFN-inducible protein (IP)-10, interleukin (IL)-12p40, IL-15 and tumor necrosis factor TNF-α in patients at day 4 after treatment. FIG. 18B as an updated FIG. 6B shows increased levels of IFN-γ, IL-12p40, IL-15, IFN-inducible protein (IP)-10, and TNFα in patients on the 4^{th} day after treatment with Construct 1 monotherapy at DL 1 (5×10⁵ RCV FFU) or DL2 (5×10⁶ RCV) or Construct 2/Construct 1 alternating 2-vector therapy (Construct 2: 1×10⁶ RCV FFU, Construct 1: 5×10⁶ RCV FFU).

In addition, the induction of antigen specific T cell responses was measured by ELISpot and intracellular cytokine staining. Whereas it is common in the field to measure the immunogenicity of cancer therapeutics by IFN-γ ELISpot only after in-vitro stimulation (IVS) of 4-14 days to expand T cells and increase the probability of detecting antigen specific T cells, the induction of antigen specific T cell responses after treatment with arenaviral vector therapy was directly measured without prior in vitro expansion. Specifically, PBMCs from patients with available samples were selected for T cell analysis by IFN-γ ELISpot and intracellular cytokine staining (ICS).

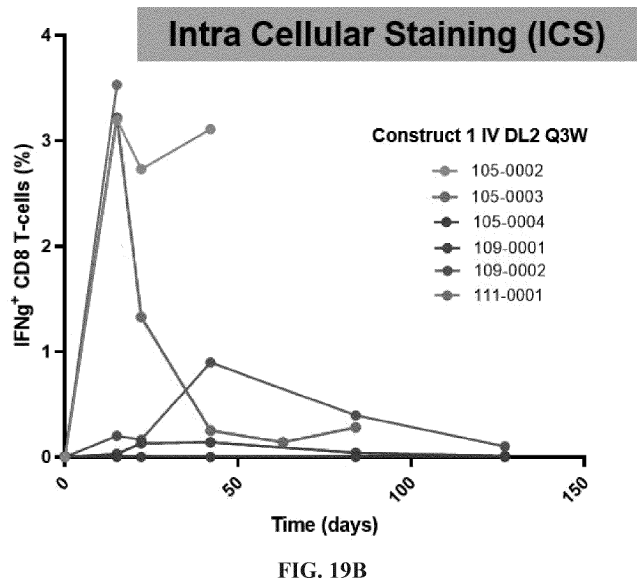

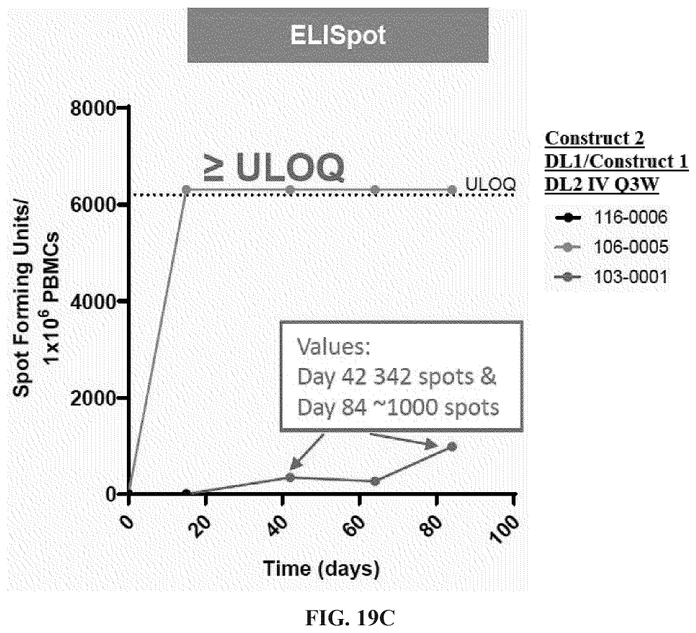

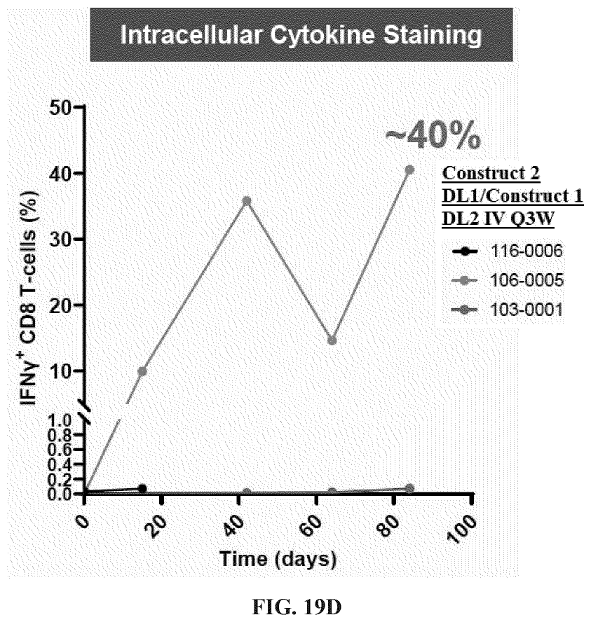

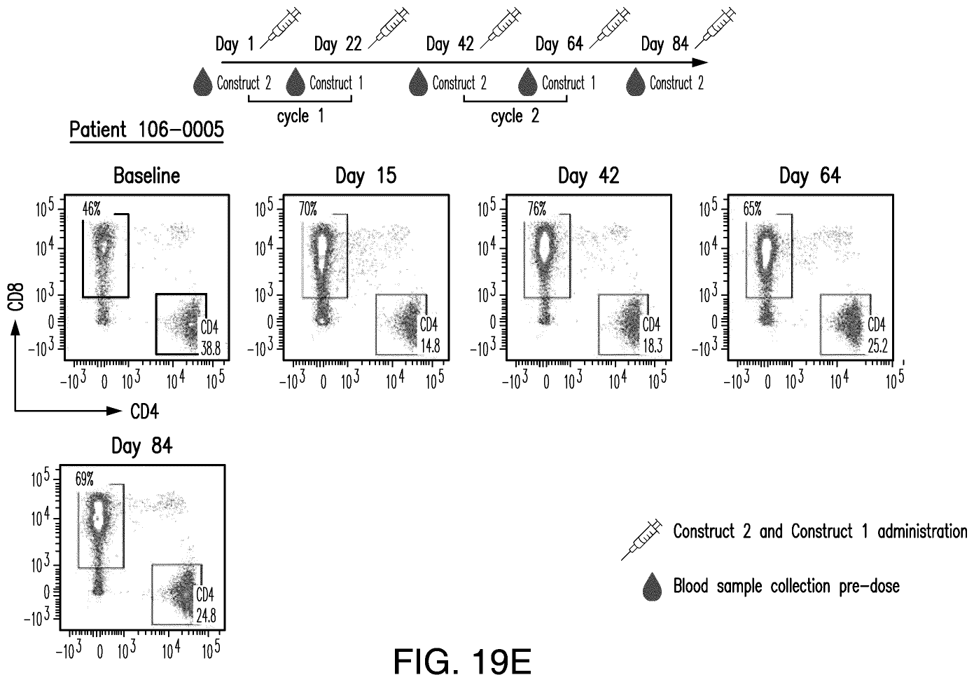

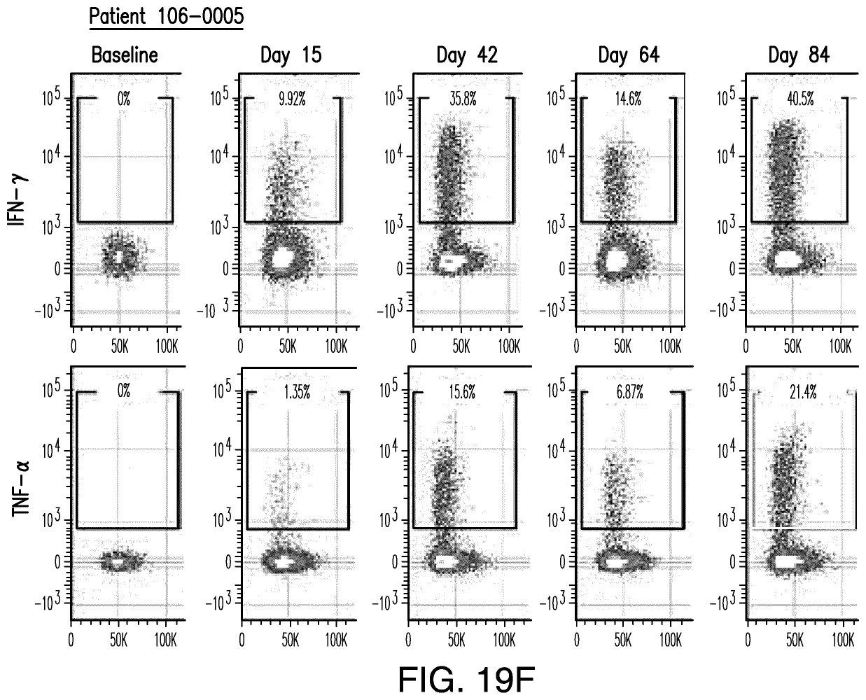

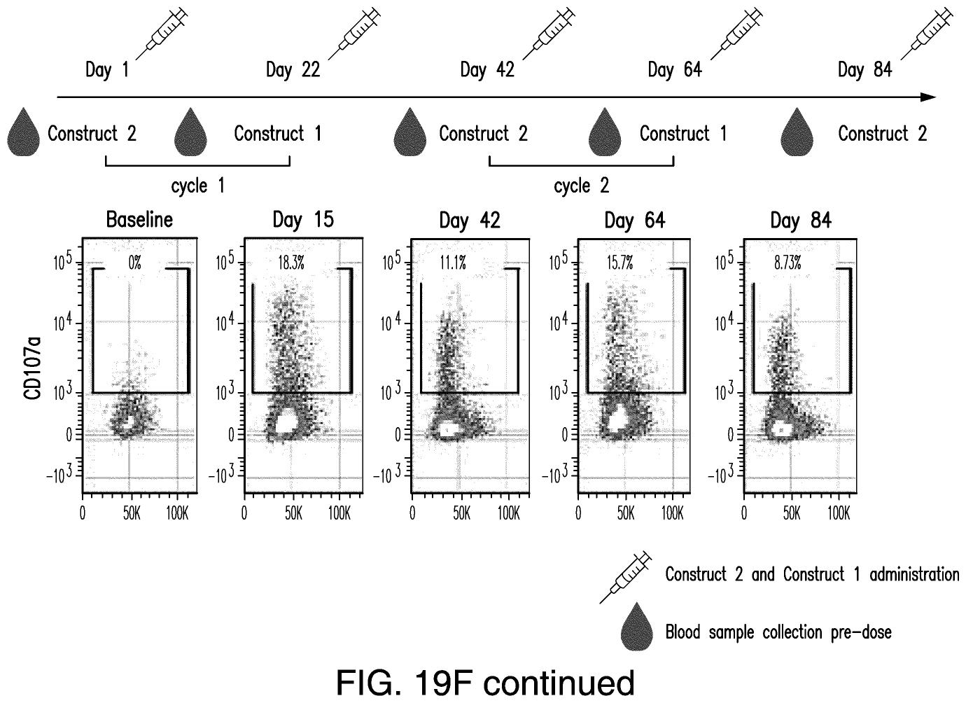

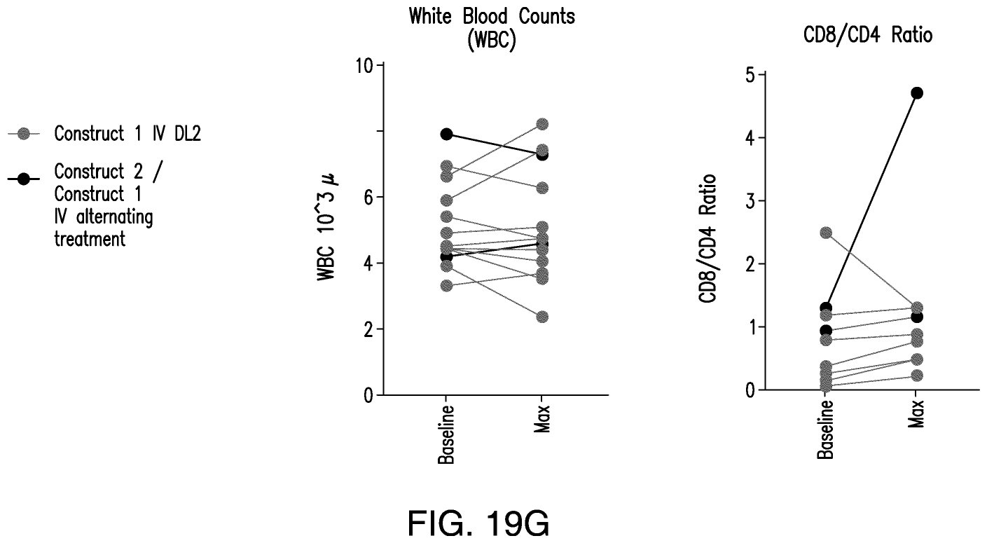

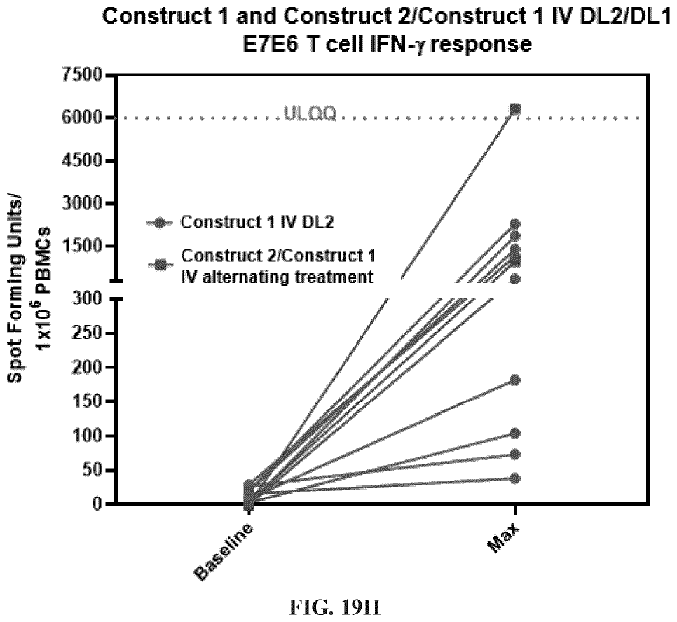

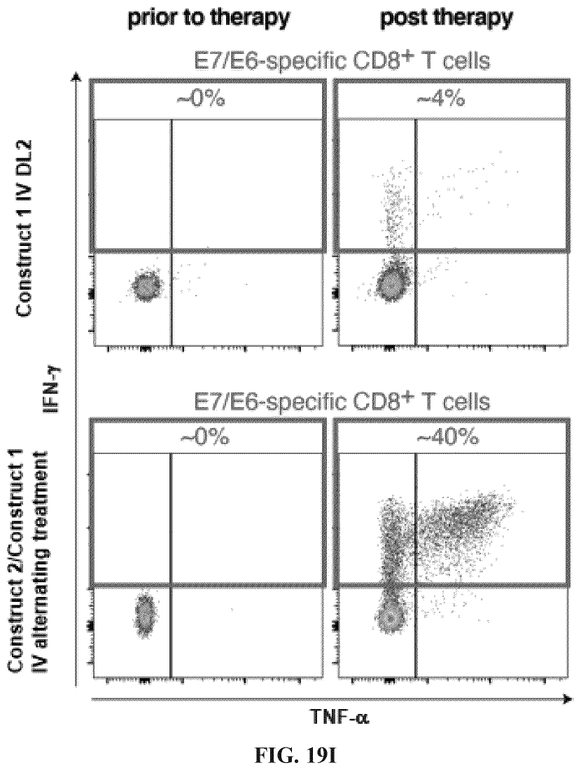

As illustrated in FIG. 19A and FIG. 19B, up to 3.5% IFN-γ⁺E6/E7 specific CD8 T cells were detected after the first dose of Construct 1 at dose level 2 (i.e., 5×10⁶ RCV FFU).

Surprisingly, unprecedented E6/E7-specific CD8 T cell levels were observed in one patient (patient 106-0005) following intravenous Construct 2/Construct 1 alternating 2-vector therapy. As shown in FIG. 19C and FIG. 19D, about 10% antigen specific CD8+ T cells were observed after a single dose of Construct 2 and up to about 40% IFN-γ+E6/E7 specific CD8 T cells could be detected after 2 cycles of Construct 2/Construct 1 alternating 2-vector therapy. For a selected patient (patient 106-0005), multicolor flow cytometry was carried out at different time points throughout multiple cycles of treatment of Construct 2/Construct 1 alternating 2-vector therapy. FIG. 19E shows an increase of circulating total CD8⁺ T cells. FIG. 19F shows an increase in functional and cytotoxic E6/E7-specific CD8 T Cells that expressed IFN-γ, TNFα, or CD107a in the same patient. Production of IFN-γ, TNFα, or CD107a demonstrates that induced E6/E7-specific CD8 T cells are multifunctional and not exhausted.

FIG. 19G to FIG. 19I further show that treatment with either Construct 1 alone or Construct 2/Construct 1 alternating 2-vector therapy induces substantial antigen-specific T cell responses in patients, with up to 40% of circulating CD8+ T cells being E6/E7 specific. IFN-γ and TNF-α production indicates that the respective T cells are not exhausted. Furthermore, expansion of E6/E7-specific CD8+ T cells in patients mirror the results observed in murine models.

In summary, Construct 1 and Construct 2 induced type 1 cytokine secretion in serum. Direct IFN-γ ELISpot and ICS performed without prior in-vitro expansion captured high-magnitude T-cell responses. Single doses of Construct 1 and Construct 2, respectively were capable of driving strong E6/E7 specific CD8 T cells with up to 9.9% IFN-γ⁺ CD8 T cells. For example, three patients after a single dose of Construct 1 produced more than 3% antigen specific CD8⁺ T cells. One patient after a single dose of Construct 2 responded with about 10% antigen specific CD8⁺ T cells. 83% of IV dosed patients receiving either Construct 1 alone or Construct 2/Construct 1 alternating 2-vector therapy demonstrated an induction of tumor-antigen specific T cell responses to HPV16 E7/E6 measured by ELISpot and/or ICS, as well as a shift to an IFN-γ signature. In view of preliminary efficacy measurement observed for HPV16⁺ HNSCC patients, Construct 1 single vector administered every 3 weeks resulted in an overall response rate (ORR) of 18% (1 partial response (PR), 1 unconfirmed complete response (uCR))

What is claimed is:

1. A method for treating cancer in a patient in need thereof comprising administering to the patient an effective amount of engineered replication-competent tri-segmented arenavirus particles comprising two S-segments encoding a fusion protein of human papillomavirus strain 16 (HPV16) E7/E6, wherein the effective amount is about 5×10⁵ replication-competent virus focus-forming units (RCV FFU).

2. A method for treating cancer in a patient in need thereof comprising administering to the patient an effective amount of engineered replication-competent tri-segmented arenavirus particles comprising two S-segments encoding a fusion protein of human papillomavirus strain 16 (HPV16) E7/E6, wherein the effective amount is about 5×10⁶ replication-competent virus focus-forming units (RCV FFU).

3. A method for treating cancer in a patient in need thereof comprising administering to the patient an effective amount of engineered replication-competent tri-segmented arenavirus particles comprising two S-segments encoding a fusion protein of human papillomavirus strain 16 (HPV16) E7/E6, wherein the effective amount is about 5×10⁷ replication-competent virus focus-forming units (RCV FFU).

4. A method for treating cancer in a patient in need thereof comprising administering to the patient an effective amount of engineered replication-competent tri-segmented arenavirus particles comprising two S-segments encoding a fusion protein of human papillomavirus strain 16 (HPV16) E7/E6, wherein the effective amount is about 5×10⁸ replication-competent virus focus-forming units (RCV FFU).

5. A method for treating cancer in a patient in need thereof comprising administering to the patient an effective amount of engineered replication-competent tri-segmented arenavirus particles comprising two S-segments encoding a fusion protein of human papillomavirus strain 16 (HPV16) E7/E6, wherein the effective amount is about 1×10⁸ replication-competent virus focus-forming units (RCV FFU).

6. The method of any one of claims 1 to 5, wherein the cancer is HPV 16⁺.

7. The method of claim 6, wherein the HPV 16⁺ cancer is head and neck squamous cell carcinoma.

8. The method of claim 6, wherein the HPV 16⁺ cancer is anal cancer, cervical cancer, vulvar, or vaginal cancer.

9. The method of any one of claims 1 to 8, wherein the patient had tumor progression or recurrence on at least one standard-of-care therapy prior to the method.

10. The method of claim 9, wherein the at least one standard-of-care therapy comprises pembrolizumab monotherapy.

11. The method of any one of claims 1 to 10, wherein the patient has only target lesions in lymph nodes.

12. The method of any one of claims 1 to 11, wherein the administration of the engineered replication-competent tri-segmented arenavirus particle comprises intravenous injections.

13. The method of claim 12, the intravenous injections are administered with a frequency of every 2 weeks, every 3 weeks, every 4 weeks, every 5 weeks, every 6 weeks, every 7 weeks, or every 8 weeks.

14. The method of claim 12 or 13, wherein the intravenous injections are ongoing or are administered for a limited number of cycles.

15. The method of claim 14, wherein the limited number of cycles is two, three, four, five, or six.

16. The method of claim 15, wherein the effective amount of the engineered replication-competent tri-segmented arenavirus particle is one log order more than the effective amounts used in the ongoing intravenous injections.

17. The method of claim 14, wherein the intravenous injections are ongoing and are first administered with a higher frequency followed by a lower frequency.

18. The method of claim 17, wherein the intravenous injections are ongoing and are first administered with a frequency of every 3 weeks followed by a frequency of every 6 weeks.

19. The method of claim 18, wherein the intravenous injection are ongoing and are first administered with a frequency of every 3 weeks for 4 cycles followed by a frequency of every 6 weeks for subsequent cycles.

20. The method of claim 17, wherein the intravenous injection are ongoing and are first administered with a frequency of every 4 weeks followed by a frequency of every 8 weeks.

21. The method of claim 20, wherein the intravenous injection are ongoing and are first administered with a frequency of every 4 weeks for 4 cycles followed by a frequency of every 8 weeks for subsequent cycles.

22. The method of any one of claims 12 to 21, wherein the method further comprises an intratumoral injection prior to the intravenous injections.

23. The method of any one of claims 1 to 11, wherein the administration of the engineered replication-competent tri-segmented arenavirus particle comprises intratumoral injections.

24. The method of any one of claims 1 to 23, wherein the method further comprises administering an effective amount of an immune checkpoint inhibitor.

25. The method of claim 24, wherein the immune checkpoint inhibitor comprises an anti-PD-1 (programmed cell death protein 1) checkpoint inhibitor.

26. The method of claim 25, wherein the anti-PD-1 checkpoint inhibitor is an antibody.

27. The method of claim 26, wherein the antibody is nivolumab, pembrolizumab, pidilizumab or cemiplimab.

28. The method of any one of claims 1 to 27, wherein the engineered replication-competent tri-segmented arenavirus particles are derived from lymphocytic choriomeningitis virus (LCMV).

29. The method of claim 28, wherein the LCMV is MP strain, WE strain, Armstrong strain, Armstrong Clone 13 strain, or LCMV clone 13 strain expressing the glycoprotein of LCMV strain WE instead of endogenous LCMV clone 13 glycoprotein.

30. The method of claim 29, wherein the engineered replication-competent tri-segmented arenavirus particles comprise Construct 1.

31. The method of claim 30, wherein the effective amount of Construct 1 is about 5×10⁶ RCV FFU, and wherein Construct 1 is administered with a frequency of every 3 weeks.

32. The method of any one of claims 1 to 27, wherein the engineered replication-competent tri-segmented arenavirus particles are derived from Pichinde virus (PICV).

33. The method of claim 32, wherein the PICV is strain Munchique CoAn4763 isolate P18, or P2 strain.

34. The method of claim 33, wherein the engineered replication-competent tri-segmented arenavirus particles comprise Construct 2.

35. The method of any one of claims 1 to 34, wherein the method results in a change in level of a cytokine or a chemokine in the serum of the patient as compared to the pre-treatment level of the patient.

36. The method of claim 35, wherein the cytokine and chemokine comprises IFN-γ, IL-12p40, IL-15, IFN-inducible protein (IP)-10, and TNFα.

37. The method of any one of claims 1 to 36, wherein the method results in an increase of HPV16 E7/E6-specific T cells in the serum of the patient as compared to the pre-treatment level of the patient.

38. The method of claim 37, wherein the HPV16 E7/E6-specific T cells are positive for CD8, IFN-γ, TNFα, and/or CD107a.

39. The method of claim 37 or 38, wherein the T cells are detected without prior in-vitro stimulation and/or expansion.

40. The method of any one of claims 1 to 39, wherein the method results in more T cells infiltrating into tumor tissues as compared to the pre-treatment level of the patient or patients receiving placebo.

41. The method of any one of claims 1 to 40, wherein the method results in one or more improved efficacy endpoint using Response Evaluation Criteria in Solid Tumors (RECIST) and/or Immune Response Evaluation Criteria in Solid Tumors (iRECIST), compared to the pre-treatment level of the patient or patients receiving placebo.

42. The method of claim 41, wherein the one or more improved efficacy endpoint comprises higher percentage of objective response rate, higher percentage of disease control rate, higher percentage of partial response, longer progression-free survival, and/or longer overall survival.

43. A method for treating cancer in a patient in need thereof comprising one or more session, wherein each session comprises
i. administering to the patient an effective amount of engineered replication-competent tri-segmented arenavirus particles comprising two S-segments encoding a fusion protein of human papillomavirus strain 16 (HPV16) E7/E6 derived from a first arenavirus species, wherein the effective amount is about 5×10⁵, 1×10⁶, 5×10⁶, 1×10⁷, 5×10⁷, 1×10⁸, 5×10⁸, or 1×10⁹ replication-competent virus focus-forming units (RCV FFU); and
ii. administering to the patient an effective amount of engineered replication-competent tri-segmented arenavirus particles comprising two S-segments encoding a fusion protein of HPV16 E7/E6 derived from a second arenavirus species at a time point around half of the session, wherein the effective amount is about 5×10⁵, 1×10⁶, 5×10⁶, 1×10⁷, 5×10⁷, 1×10⁸, 5×10⁸, or 1×10⁹ RCV FFU.

44. The method of claim 43, wherein the first arenavirus species in (i) is lymphocytic choriomeningitis virus (LCMV), and the second arenavirus species in (ii) is Pichinde virus (PICV).

45. The method of claim 43, wherein the first arenavirus species in (i) is PICV, and the second arenavirus species in (ii) is LCMV.

46. The method of claim 44 or 45, wherein the LCMV is MP strain, WE strain, Armstrong strain, Armstrong Clone 13 strain, or LCMV clone 13 strain expressing the glycoprotein of LCMV strain WE instead of endogenous LCMV clone 13 glycoprotein.

47. The method of claim 46, wherein the engineered replication-competent tri-segmented arenavirus particles comprise Construct 1.

48. The method of claim 44 or 45, wherein the PICV is strain Munchique CoAn4763 isolate P18, or P2 strain.

49. The method of claim 48, wherein the engineered replication-competent tri-segmented arenavirus particles comprise Construct 2.

50. The method of claim 45, wherein the engineered replication-competent tri-segmented arenavirus particles in (i) are Construct 2, and the engineered replication-competent tri-segmented arenavirus particles in (ii) are Construct 1.

51. The method of claim 50 comprising one or more session, wherein each session comprises:
i. administering to the patient an effective amount of Construct 2, wherein the effective amount is about 1×10⁶, 1×10⁷, 1×10⁸, or 1×10⁹ RCV FFU; and
ii. administering to the patient an effective amount of Construct 1 at a time point around half of the session, wherein the effective amount is about 5×10⁵, 5×10⁶, 5×10⁷, 1×10⁸, or 5×10⁸ RCV FFU.

52. The method of claim 51, wherein the effective amount of Construct 2 is about 1×10⁶ RCV FFU and the effective amount of Construct 1 is about 5×10⁶ RCV FFU, and wherein Construct 2 and Construct 1 are administered intravenously, and each session lasts for 6 weeks.

53. The method of any one of claims 43 to 52, wherein the cancer is HPV 16⁺.

54. The method of claim 53, wherein the HPV 16⁺ cancer is head and neck squamous cell carcinoma.

55. The method of claim 53, wherein the HPV 16⁺ cancer is anal cancer, cervical cancer, vulvar, or vaginal cancer.

56. The method of any one of claims 43 to 55, wherein the patient had tumor progression or recurrence on at least one standard-of-care therapy prior to the method.

57. The method of claim 56, wherein the at least one standard-of-care therapy comprises pembrolizumab monotherapy.

58. The method of any one of claims 43 to 57, wherein the patient has only target lesions in lymph nodes.

59. The method of any one of claims 43 to 58, wherein the administration of the engineered replication-competent tri-segmented arenavirus particles in (i) and (ii) comprises intravenous injection.

60. The method of claim 59, wherein each session lasts for 4 weeks, 6 weeks, 8 weeks, 10 weeks, 12 weeks, 14 weeks, or 16 weeks.

61. The method of claim 59 or 60, wherein the sessions are ongoing or are repeated for a limited number of sessions.

62. The method of claim 61, wherein the limited number of sessions is two, three, four, five, or six.

63. The method of claim 62, wherein the effective amount of the engineered replication-competent tri-segmented arenavirus particles is one log order more than the effective amount used in the ongoing sessions.

64. The method of claim 61, wherein the intravenous injections are ongoing and are first administered in shorter sessions followed by longer sessions.

65. The method of claim 64, wherein the intravenous injection are ongoing and are first administered with sessions each lasting 6 weeks followed by sessions each lasting 12 weeks.

66. The method of claim 65, wherein the intravenous injections are ongoing and are first administered with 2 sessions each lasting 6 weeks followed by sessions each lasting 12 weeks.

67. The method of claim 64, wherein the intravenous injections are ongoing and are first administered with sessions each lasting 8 weeks followed by sessions each lasting 16 weeks.

68. The method of claim 67, wherein the intravenous injection are ongoing and are first administered with 2 sessions each lasting 8 weeks followed by sessions each lasting 16 weeks.

69. The method of any one of claims 59 to 68, wherein the method further comprises an intratumoral injection prior to the intravenous injections.

70. The method of claim 69, wherein the intratumoral injection is administered 3 weeks prior to the intravenous injections.

71. The method of claim 69 or 70, wherein the intratumoral injection is administered with Construct 1.

72. The method of any one of claims 43 to 58, wherein the administration of the engineered replication-competent tri-segmented arenavirus particle comprises intratumoral injections.

73. The method of any one of claims 43 to 72, wherein the method further comprises administering an effective amount of an immune checkpoint inhibitor.

74. The method of claim 73, wherein the immune checkpoint inhibitor comprises an anti-PD-1 (programmed cell death protein 1) checkpoint inhibitor.

75. The method of claim 74, wherein the anti-PD-1 checkpoint inhibitor is an antibody.

76. The method of claim 75, wherein the antibody is nivolumab, pembrolizumab, pidilizumab or cemiplimab.

77. The method of any one of claims 43 to 76, wherein the method results in a change in level of a cytokine or a chemokine in the serum of the patient as compared to the pre-treatment level of the patient.

78. The method of claim 77, wherein the cytokine and chemokine comprises IFN-γ, IL-12p40, IL-15, IFN-inducible protein (IP)-10, and TNFα.

79. The method of any one of claims 43 to 78, wherein the method results in an increase of HPV16 E7/E6-specific T cells in the serum of the patient as compared to the pre-treatment level of the patient.

80. The method of claim 79, wherein the HPV16 E7/E6-specific T cells are positive for CD8, IFN-γ, TNFα, and/or CD107a.

81. The method of claim 79 or 80, wherein the T cells are detected without prior in-vitro stimulation and/or expansion.

82. The method of any one of claims 43 to 81, wherein the method results in more T cells infiltrating into tumor tissues as compared to the pre-treatment level of the patient or patients receiving placebo.

83. The method of any one of claims 43 to 82, wherein the method results in one or more improved efficacy endpoint using Response Evaluation Criteria in Solid Tumors (RECIST) and/or Immune Response Evaluation Criteria in Solid Tumors (iRECIST).

84. The method of claim 83, wherein the one or more improved efficacy endpoint comprises higher percentage of objective response rate, higher percentage of disease control rate, higher percentage of partial response, longer progression-free survival, longer overall survival.

85. A method for treating cancer in a patient in need thereof comprising administering to the patient an effective amount of Construct 1, wherein the effective amount is about 5×10⁵, 5×10⁶, 5×10⁷, 1×10⁸, or 5×10⁸ replication-competent virus focus-forming units (RCV FFU), and wherein Construct 1 is administered intravenously with a frequency of every 3 weeks for 4 cycles followed by ongoing cycles with a frequency of every 6 weeks.

86. A method for treating cancer in a patient in need thereof comprising
(i) administering to the patient an effective amount of Construct 1, wherein the effective amount is about 5×10⁵, 5×10⁶, 5×10⁷, 1×10⁸, or 5×10⁸ replication-competent virus focus-forming units (RCV FFU), and wherein Construct 1 is administered intravenously with a frequency of every 3 weeks for 4 cycles followed by ongoing cycles with a frequency of every 6 weeks; and
(ii) administering to the patient 200 mg of pembrolizumab intravenously with a frequency of every 3 weeks or 400 mg of pembrolizumab intravenously with a frequency of every 6 weeks.

87. A method for treating cancer in a patient in need thereof comprising multiple sessions, wherein each session comprises
i. administering to the patient an effective amount of Construct 2 intravenously, wherein the effective amount is about 1×10⁶ replication-competent virus focus-forming units (RCV FFU); and
ii. administering to the patient an effective amount of Construct 1 intravenously at a time point around half of the session, wherein the effective amount is about 5×10⁶ RCV FFU,
and wherein the first two sessions each lasts for 6 weeks, and the following ongoing sessions each lasts for 12 weeks.

88. A method for treating cancer in a patient in need thereof comprising multiple sessions, wherein each session comprises
i. administering to the patient an effective amount of Construct 2 intravenously, wherein the effective amount is about 1×10⁷ replication-competent virus focus-forming units (RCV FFU); and
ii. administering to the patient an effective amount of Construct 1 intravenously at a time point around half of the session, wherein the effective amount is about 5×10⁶ RCV FFU,
and wherein the first two sessions each lasts for 6 weeks, and the following ongoing sessions each lasts for 12 weeks.

89. A method for treating cancer in a patient in need thereof comprising multiple sessions, wherein each session comprises
i. administering to the patient an effective amount of Construct 2 intravenously, wherein the effective amount is about 1×10⁷ replication-competent virus focus-forming units (RCV FFU); and
ii. administering to the patient an effective amount of Construct 1 intravenously at a time point around half of the session, wherein the effective amount is about 5×10⁷ RCV FFU,
and wherein the first two sessions each lasts for 6 weeks, and the following ongoing sessions each lasts for 12 weeks.

90. A method for treating cancer in a patient in need thereof comprising multiple sessions, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 5×10⁷ RCV FFU,
and wherein the first two sessions each lasts for 6 weeks, and the following ongoing sessions each lasts for 12 weeks.

91. A method for treating cancer in a patient in need thereof comprising multiple sessions, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 1×10⁸ RCV FFU,
and wherein the first two sessions each lasts for 6 weeks, and the following ongoing sessions each lasts for 12 weeks.

92. A method for treating cancer in a patient in need thereof comprising multiple sessions, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 5×10⁸ RCV FFU,
and wherein the first two sessions each lasts for 6 weeks, and the following ongoing sessions each lasts for 12 weeks.

93. A method for treating cancer in a patient in need thereof comprising
(1) multiple sessions of administering Construct 2 and Construct 1, wherein each session comprises
i. administering to the patient an effective amount of Construct 2 intravenously, wherein the effective amount is about 1×10⁶ replication-competent virus focus-forming units (RCV FFU); and
ii. administering to the patient an effective amount of Construct 1 intravenously at a time point around half of the session, wherein the effective amount is about 5×10⁶ RCV FFU,
and wherein the first two sessions each lasts for 6 weeks, and the following ongoing sessions each lasts for 12 weeks; and
(2) administering to the patient 200 mg of pembrolizumab intravenously at a frequency of every 3 weeks or 400 mg of pembrolizumab intravenously at a frequency of every 6 weeks.

94. A method for treating cancer in a patient in need thereof comprising
(1) multiple sessions of administering Construct 2 and Construct 1, wherein each session comprises
i. administering to the patient an effective amount of Construct 2 intravenously, wherein the effective amount is about 1×10⁷ replication-competent virus focus-forming units (RCV FFU); and
ii. administering to the patient an effective amount of Construct 1 intravenously at a time point around half of the session, wherein the effective amount is about 5×10⁶ RCV FFU,
and wherein the first two sessions each lasts for 6 weeks, and the following ongoing sessions each lasts for 12 weeks; and
(2) administering to the patient 200 mg of pembrolizumab intravenously at a frequency of every 3 weeks or 400 mg of pembrolizumab intravenously at a frequency of every 6 weeks.

95. A method for treating cancer in a patient in need thereof comprising
(1) multiple sessions of administering Construct 2 and Construct 1, wherein each session comprises
i. administering to the patient an effective amount of Construct 2 intravenously, wherein the effective amount is about 1×10⁷ replication-competent virus focus-forming units (RCV FFU); and
ii. administering to the patient an effective amount of Construct 1 intravenously at a time point around half of the session, wherein the effective amount is about 5×10⁷ RCV FFU,
and wherein the first two sessions each lasts for 6 weeks, and the following ongoing sessions each lasts for 12 weeks; and
(2) administering to the patient 200 mg of pembrolizumab intravenously at a frequency of every 3 weeks or 400 mg of pembrolizumab intravenously at a frequency of every 6 weeks.

96. A method for treating cancer in a patient in need thereof comprising
(1) multiple sessions of administering Construct 2 and Construct 1, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 5×10⁷ RCV FFU,
and wherein the first two sessions each lasts for 6 weeks, and the following ongoing sessions each lasts for 12 weeks; and
(2) administering to the patient 200 mg of pembrolizumab intravenously at a frequency of every 3 weeks or 400 mg of pembrolizumab intravenously at a frequency of every 6 weeks.

97. A method for treating cancer in a patient in need thereof comprising
(1) multiple sessions of administering Construct 2 and Construct 1, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 1×10⁸ RCV FFU,
and wherein the first two sessions each lasts for 6 weeks, and the following ongoing sessions each lasts for 12 weeks; and
(2) administering to the patient 200 mg of pembrolizumab intravenously at a frequency of every 3 weeks or 400 mg of pembrolizumab intravenously at a frequency of every 6 weeks.

98. A method for treating cancer in a patient in need thereof comprising
(1) multiple sessions of administering Construct 2 and Construct 1, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 5×10⁸ RCV FFU,
and wherein the first two sessions each lasts for 6 weeks, and the following ongoing sessions each lasts for 12 weeks; and
(2) administering to the patient 200 mg of pembrolizumab intravenously at a frequency of every 3 weeks or 400 mg of pembrolizumab intravenously at a frequency of every 6 weeks.

99. A method for treating cancer in a patient in need thereof comprising
(1) administering intratumorally to the patient an effective amount of Construct 1, wherein the effective amount of Construct 1 is about 5×10⁶ replication-competent virus focus-forming units (RCV FFU); and
(2) 3 weeks later administering to the patient multiple sessions, wherein each session comprises
i. administering intravenously to the patient an effective amount of Construct 2, wherein the effective amount is about 1×10⁶ RCV FFU; and
ii. administering intravenously to the patient an effective amount of Construct 1 at a time point around half of the session, wherein the effective amount is about 5×10⁶ RCV FFU,
and wherein the first two sessions each lasts for 6 weeks, and the following ongoing sessions each lasts for 12 weeks.

100. A method for treating cancer in a patient in need thereof comprising
(1) administering intratumorally to the patient an effective amount of Construct 1, wherein the effective amount of Construct 1 is about 5×10⁶ replication-competent virus focus-forming units (RCV FFU); and
(2) 3 weeks later administering to the patient multiple sessions, wherein each session comprises
i. administering intravenously to the patient an effective amount of Construct 2, wherein the effective amount is about 1×10⁷ RCV FFU; and
ii. administering intravenously to the patient an effective amount of Construct 1 at a time point around half of the session, wherein the effective amount is about 5×10⁶ RCV FFU,
and wherein the first two sessions each lasts for 6 weeks, and the following ongoing sessions each lasts for 12 weeks.

101. A method for treating cancer in a patient in need thereof comprising
(1) administering intratumorally to the patient an effective amount of Construct 1, wherein the effective amount of Construct 1 is about 5×10⁷ replication-competent virus focus-forming units (RCV FFU); and
(2) 3 weeks later administering to the patient multiple sessions, wherein each session comprises
i. administering intravenously to the patient an effective amount of Construct 2, wherein the effective amount is about 1×10⁷ RCV FFU; and
ii. administering intravenously to the patient an effective amount of Construct 1 at a time point around half of the session, wherein the effective amount is about 5×10⁷ RCV FFU,
and wherein the first two sessions each lasts for 6 weeks, and the following ongoing sessions each lasts for 12 weeks.

102. A method for treating cancer in a patient in need thereof comprising
(1) administering intratumorally to the patient an effective amount of Construct 1, wherein the effective amount of Construct 1 is about 5×10⁷ replication-competent virus focus-forming units (RCV FFU); and
(2) 3 weeks later administering to the patient multiple sessions, wherein each session comprises
i. administering intravenously to the patient an effective amount of Construct 2, wherein the effective amount is about 1×10⁸ RCV FFU; and
ii. administering intravenously to the patient an effective amount of Construct 1 at a time point around half of the session, wherein the effective amount is about 5×10⁷ RCV FFU,
and wherein the first two sessions each lasts for 6 weeks, and the following ongoing sessions each lasts for 12 weeks.

103. A method for treating cancer in a patient in need thereof comprising
(1) administering intratumorally to the patient an effective amount of Construct 1, wherein the effective amount of Construct 1 is about 1×10⁸ replication-competent virus focus-forming units (RCV FFU); and
(2) 3 weeks later administering to the patient multiple sessions, wherein each session comprises
i. administering intravenously to the patient an effective amount of Construct 2, wherein the effective amount is about 1×10⁸ RCV FFU; and
ii. administering intravenously to the patient an effective amount of Construct 1 at a time point around half of the session, wherein the effective amount is about 1×10⁸ RCV FFU,
and wherein the first two sessions each lasts for 6 weeks, and the following ongoing sessions each lasts for 12 weeks.

104. A method for treating cancer in a patient in need thereof comprising
(1) administering intratumorally to the patient an effective amount of Construct 1, wherein the effective amount of Construct 1 is about 5×10⁸ replication-competent virus focus-forming units (RCV FFU); and
(2) 3 weeks later administering to the patient multiple sessions, wherein each session comprises
i. administering intravenously to the patient an effective amount of Construct 2, wherein the effective amount is about 1×10⁸ RCV FFU; and
ii. administering intravenously to the patient an effective amount of Construct 1 at a time point around half of the session, wherein the effective amount is about 5×10⁸ RCV FFU,
and wherein the first two sessions each lasts for 6 weeks, and the following ongoing sessions each lasts for 12 weeks.

105. A method for treating cancer in a patient in need thereof comprising administering to the patient an effective amount of Construct 1, wherein the effective amount is about 5×10⁶, 5×10⁷, 5×10⁸, 1×10⁹, or 5×10⁹ replication-competent virus focus-forming units (RCV FFU), and wherein Construct 1 is administered intravenously with a frequency of every 3 weeks for 3 cycles and the method ends after 3 cycles.

106. A method for treating cancer in a patient in need thereof comprising 3 sessions, wherein each session comprises
i. administering to the patient an effective amount of Construct 2 intravenously, wherein the effective amount is about 1×10⁷ replication-competent virus focus-forming units (RCV FFU); and
ii. administering to the patient an effective amount of Construct 1 intravenously at a time point around half of the session, wherein the effective amount is about 5×10⁷ RCV FFU,
and wherein each sessions lasts for 6 weeks, and the method ends after 3 sessions.

107. A method for treating cancer in a patient in need thereof comprising 3 sessions, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 5×10⁷ RCV FFU,
and wherein each sessions lasts for 6 weeks, and the method ends after 3 sessions.

108. A method for treating cancer in a patient in need thereof comprising 3 sessions, wherein each session comprises
i. administering to the patient an effective amount of Construct 2 intravenously, wherein the effective amount is about 1×10⁸ replication-competent virus focus-forming units (RCV FFU); and
ii. administering to the patient an effective amount of Construct 1 intravenously at a time point around half of the session, wherein the effective amount is about 5×10⁸ RCV FFU,
and wherein each sessions lasts for 6 weeks, and the method ends after 3 sessions.

109. A method for treating cancer in a patient in need thereof comprising 3 sessions, wherein each session comprises
i. administering to the patient an effective amount of Construct 2 intravenously, wherein the effective amount is about 1×10⁹ replication-competent virus focus-forming units (RCV FFU); and
ii. administering to the patient an effective amount of Construct 1 intravenously at a time point around half of the session, wherein the effective amount is about 5×10⁸ RCV FFU,
and wherein each sessions lasts for 6 weeks, and the method ends after 3 sessions.

110. A method for treating cancer in a patient in need thereof comprising 3 sessions, wherein each session comprises
i. administering to the patient an effective amount of Construct 2 intravenously, wherein the effective amount is about 1×10⁹ replication-competent virus focus-forming units (RCV FFU); and
ii. administering to the patient an effective amount of Construct 1 intravenously at a time point around half of the session, wherein the effective amount is about 1×10⁹ RCV FFU,
and wherein each sessions lasts for 6 weeks, and the method ends after 3 sessions.

111. A method for treating cancer in a patient in need thereof comprising 3 sessions, wherein each session comprises
i. administering to the patient an effective amount of Construct 2 intravenously, wherein the effective amount is about 1×10⁹ replication-competent virus focus-forming units (RCV FFU); and
ii. administering to the patient an effective amount of Construct 1 intravenously at a time point around half of the session, wherein the effective amount is about 5×10⁹ RCV FFU,
and wherein each sessions lasts for 6 weeks, and the method ends after 3 sessions.

112. A nucleotide sequence comprising the nucleotide sequence of SEQ ID NOs: 1 or 2.

113. A nucleotide sequence comprising the nucleotide sequence of SEQ ID NOs: 3, 4, 5, 6, 7, or 8.

114. The nucleotide sequence of claim 112 or 113, wherein the nucleotide sequence is RNA.

115. A host cell comprising the nucleotide sequence of any one of claims 112 to 114.

116. A tri-segmented LCMV particle comprising the nucleotide sequences of SEQ ID NOs: 3, 4, and 5.

117. A tri-segmented PICV particle comprising the nucleotide sequences of SEQ ID NOs: 6, 7, and 8.

118. A pharmaceutical composition comprising the tri-segmented viral particle of claim 116 or 117 and a pharmaceutically acceptable carrier.

119. The tri-segmented arenavirus particle of claim 116 or 117, wherein the dinucleotide optimized HPV16 E7E6 nucleotide sequence can:
i. have stable expression of the HPV antigen after being passaged at least 4, 5, 6, 7, 8, 9, or 10 generations;
ii. have consistent expression of the encoded HPV fusion protein; or
iii. induce strong immune responses against the encoded HPV fusion protein.


SEQUENCE LISTING (8 sequences)

SEQ ID NO: 1 - DNA, 768 bp, Artificial Sequence
  Nucleotide sequence of HPV16 E7E6
atgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtga

SEQ ID NO: 2 - DNA, 768 bp, Artificial Sequence
  Nucleotide sequence of HPV16 E7E6
atgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagtaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtga

SEQ ID NO: 3 - DNA, 2648 bp, Artificial Sequence
  Nucleotide sequence of LCMV HPV16 E7E6-NP-S- segment 1
gcgcaccggggatcctaggctttttggattgcgctttcctctagatcaactgggtgtcaggccctatcctacagaaggatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttatcttcatactccctcgaagcttccctggtcatttcaacatcgataagcttaatgtccttcctattttgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgagttgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagagatcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 4 - DNA, 2468 bp, Artificial Sequence
  Nucleotide sequence of LCMV HPV16 E7E6-GP-S- segment 2
gcgcaccggggatcctaggctttttggattgcgctttcctctagatcaactgggtgtcaggccctatcctacagaaggatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagtaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgaagaacagcgcctccctgactctccacctcgaaagaggtggagagtcagggaggcccagagggtctcagcgtcttttccagatagtttttacaccaggcaccttgaatgcaccacaactacagatccccttgttggtcaagcggtgtggctttggacatgaaccgccctttatgtgtctatgtgttggtatcttcacaagatgcagaaagatgctgattagatatgctgatgttgaaaacatcaaaagatccattaaggctaaaggagtactcccttgtctttttatgtagtccttcctcaacatctctgtgatcatgttatctgcttcttgttcgatttgatcactaaagtgggtctcattcaagtaggagccattagtgacaagccagcacttgggtacactagtctcaccagtcttagcatgttccagataccagaactttgagtaattacagtatggtacccccattagatctcttagatgattcctcatcaacagctgatcggaaatcagagaatttactgttgttttgaatacatgcaaggcagactctacatcttgcttgaacttactcagggcggccttgttgtaatcaattagtcgtagcatgtcacagaactcttcatcatgattgacattacattttgcaacagctgtattcccaaaacatttgagctctgcagcaaggatcatccatttggtcaggcaataaccacctggattttctactcctgaggagtctgacagggtccaggtgaatgtgcctgcaagtctcctagtgagaaactttgtcttttcctgagcaaagaggattctagacatcccaaaagggcctgcatatctacagtggttttcccaagtcctgttttgtatgattaggtactgatagcttgtttggctgcaccaagtggtcttgccatctgaacctgcccagccccagccacttctcatgtattttcctccaaaggcagttctaaacatgtccaagactctacctctgaaagtcctacactggcttatagcgctctgtgggtccgaaaatgacaagttgtattgaatggtgatgccattgttaaaatcacaagacactgctttgtggttggaattccctctaatactgaggtgcagactcgagactatactcatgagtgtatggtcaaaagtctttttgttgaaagcggaggttaagttgcaaaaattgtgattaaggatggagtcgttagtgaaagttagctccagtccagagcttcccatactgatgtagtgatgagagttgttggctgagcacgcattgggcatcgtcagatttaagtgagacatatcaaactccactgatttgaactggtaaacccctttatagatgtcgggaccattaaggccgtacatgccacaggacctaccagccaaaaaaaggaagctgaccagtgctaatatcccacaggtggcgaaattgtacacagctttgatgctcgtgattataatgagcacaataatgacaatgttgatgacctcatcaatgatgtgaggcaaagcctcaaacattgtcacaatctgacccatcttgttgctcaatggtttctcaagacaaatgcgcaatcaaatgcctaggatccactgtgcg

SEQ ID NO: 5 - DNA, 7229 bp, Artificial Sequence
  Nucleotide sequence of LCMV L-segment
gcgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcacccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcggtgtcctcggccaccgacccgcctagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatatagccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattgtgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgaaaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagatgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattcgactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattgtgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

SEQ ID NO: 6 - DNA, 2663 bp, Artificial Sequence
  Nucleotide sequence of PICV HPV16 E7E6-NP-S- segment 1
gcgcaccggggatcctaggcataccttggacgcgcatattacttgatcaaagatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 7 - DNA, 2504 bp, Artificial Sequence
  Nucleotide sequence of PICV HPV16 E7E6-GP-S- segment 2
gcgcaccggggatcctaggcataccttggacgcgcatattacttgatcaaagatgcatggtgacacccccaccctgcatgagtacatgctggacctgcagccagagaccacagacctgtatggctatggccagctgaatgacagcagtgaggaagaggatgagattgatgggccagcaggccaggcagaacctgacagagcccactacaacattgtcaccttctgctgcaagtgtgacagcaccctgagactgtgtgtgcagagcacccatgtggacatcagaaccctggaagacctgctgatgggcaccctgggcattgtgggccccatctgctcccagaagccccaccagaaaagaactgccatgttccaggacccccaggagaggcccagaaagctgccccagctctgcacagagctgcagaccaccatccatgacatcatcctggaatgtgtctactgcaagcagcagctgctgaggagagaggtgtatgactttgccttcagggacctgtgcattgtgtacagggatggcaacccctatgctgtgggggacaagtgcctcaagttctacagcaagatcagtgagtacaggcactactgctacagcctgtatggcaccaccctggaacagcagtacaacaagcccctgtgtgacctcctgatcagatgcatcaatggccagaaacccctctgccctgaggaaaagcagagacacctggacaagaagcagaggttccacaacatcagaggcaggtggacaggcagatgcatgagctgctgcagaagcagcagaaccagaagagagacccagctgtgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 8 - DNA, 7058 bp, Artificial Sequence
  Nucleotide sequence of PICV L-segment
gcgcaccggggatcctaggcatctttgggtcacgcttcaaatttgtccaatttgaacccagctcaagtcctggtcaaaacttgggatgggactcagatatagcaaagaggtcaggaagagacatggcgacgaagatgtggtgggaagggtccccatgaccctcaatctaccacagggcctgtatggcaggttcaactgcaaatcttgctggttcgtcaacaaaggtctcatcaggtgcaaagaccactatctgtgtcttgggtgcttaaccaaaatgcactccagaggcaatctctgcgagatatgcggccactcactgccaaccaagatggagttcctagaaagcccctctgcaccaccctacgagccataaaccagggcccctgggcgcacccccctccgggggtgcgcccgggggcccccggccccatggggccggttgtttactcgatctccactgactcattgtcctcaaacaactttcgacacctgattcccttgatcttgaagggtcctgtctcgtctgcaatcataacagatcctagagtcttacttcttattatactaaagtgaccacaattcaaccaatctttggcatcatgcaacatgtgttcaaacacttcggggaaattttcaatcatgagtcttaaatcctgctcgttcatacttattcccttgttgtgagactgtgcacttgaaaggtactgaaaaaggttggcaataaatcttggccttttctcaggttctaatgcttccagtgcaatgatgaccacctttgagtctaagttcacttccaatctagaaaccactctgttgccctctttgatcaacccaccctctaaaatgaggggttgcatcccaacatcaggaccaatcaacttataggaaaatttgtttttcaaatccttgaaacgatttttcaaatctattctcaccttctggaacacagttgaccttgacttgaagtgaatgtcttgaccttccaatagatcattgaagtctagaacatcttttccgttgatgagaggattcagaaccaaaagtgacacaccatccagacttatgtgattcccggaagattgagaaacataatactcaacagaatgggggttcaacaataggtaaccatcagagtccaatgagtccagcaatgactccctttcaataagaaatcttaattttaatatgtaattggtagacctctcatatctaaatttgtggctcactctcttatgagaaaatgttaggttgagctcaatgggaatgacctcagaaggtgatgctaaaatgagttgttcaatgttctcatagttatctctattcacccagtcaagttcattaataaatacactaatgttcaaattaacacaggacaaaatcagtttgctgcttacaaagccaacatccaagtcatccagattcattgtcctagaagtgttattctttttgcagtcacaaatgaactgggttaattgtttcagatcatgttgtgcattgtttggcaacaattcaagctcaccaaaccaaaaatatttcttgaactgagatgttgacataatcacaggcaccaacattgactcaaacaaaatctgtatcaagaaatttgtgcacacttcttctggttcaaggttgaatcctctctccagtggatgagactctctgctatgggacattgcaagctcattttgctttacaatatacaattcttctctgcgatgttttataatatgactaacaataccaagacattctgatgttatatcaattgccacacaaaggtctaagaactttatcctctgaacccatgatagcctcagcatattcaaatcagacaggaaaggggatatgtgttcatcaaatagtgtagggaagttcctcctgattgagtaaagtatgtggttgatgcccaccttgtcctcaagctcagaatgtgtgcttggttttattggccagaagtgattgggattgtttaggtgagtgactatcttgggtacttcagctttttgaaacacccagttacccaactcgcaagcattggttaacacaagagcaaaataatcccaaattaagggtctggagtactcacttacttcaccaagtgctgctttacaataaacacctttgcgctgattacaaaagtgacaatcacggtgtaagataatcttgcttgtaatatccctgatatacttaaatcctcctttcccatctcttacacattttgagcccatacttttgcaaactcctatgaatcctgatgctatgctgctctgaaaagctgatttgttgatagcatcagccaaaatcttcttagcccctctgacatagttctttgataatttggactgtacggatttgacaagactgggtatttcttctcgctgcacagttcttgttgtgctcattaacttagtacgaagcaccaatctgagatcaccatgaacccttaaatttaaccacctaatattaagagcatcctcaatagcctcagtctcgacatcacaagtctctaataactgttttaagcagtcatccggtgattgctgaagagttgttacaatataactttcttccagggctccagactgtattttgtaaaatattttcctgcatgcctttctgattattgaaagtagcagatcatcaggaaatagtgtctcaattgatcgctgaagtctgtaccctctcgacccattaacccaatcgagtacatccatttcttccaggcacaaaaatggatcatttggaaacccactatagattatcatgctatttgttcgttttgcaatggcccctacaacctctattgacaccccgttagcaacacattggtccagtattgtgtcaattgtatctgcttgctgattgggtgctttagcctttatgttgtgtagagctgcagcaacaaactttgtaaggagggggacttcttgtgaccaaatgaagaatctcgatttgaactcacttgcaaaggtccccacaactgttttagggctcacaaacttgttgagtttgtctgatagaaagtagtgaaactccatacagtccaataccaattcaacattcaactcatctctgtccttaaatttgaaaccctcattcaaggataacatgatctcatcatcactcgaagtatatgagatgaaccgtgctccataacaaagctccaatgcgtaattgatgaactgctcagtgattagaccatataagtcagaggtgttgtgtaggatgccctgacccatatctaagactgaagagatgtgtgatggtaccttgcccttctcaaagtacccaaacataaattcctctgcaattgtgcacccccctttatccatcatacccaacccccttttcaagaaacctttcatgtatgcctcaacgacattgaagggcacttccaccatcttgtgaatgtgccatagcaatatgttgatgactgcagcattgggaacttctgacccatctttgagtttgaactcaagaccttttaataatgcggcaaagataaccggcgacatgtgtggcccccattttgaatggtccattgacaccgcaagaccactttgcctaacaactgacttcatgtctaataatgctctctcaaactctttctcgttgttcagacaagtatacctcatgttttgcataagggattcagagtaatcctcaatgagtctggttgtgagtttagtatttaaatcaccgacataaagctccctgttgccacccacctgttctttataagaaagaccaaatttcaatctccctacattggtggatacaccagacctctctgtgggagactcatctgaatagaaacagagatttcgtaaggatgagttggtaaaaaagctttgatccaatcttttagctatcgattcagaattgctctctcttgagcttatacgtgatgtctctctaatttgtagtgctgcatctgtgaacccaagtctgcttctacttttgtgatcatatcttccgactcgattatcataatcgcttgcaatgagaatgtatttaaagcactcaaaataatcagcttctttgtacgccttcaatgtgaggttctttattaaaaactccagaggacacggattcattagtctgtctgcaaagtacactgatctagcagtgacatcctcatagatcaagtttacaagatcctcatacacttctgctgaaaacaggctgtaatcaaaatcctttacatcatgaagtgaagtctctcttttgatgacaaccattgtcgatttgggccataatctctctagtggacatgaagtcttaaggttggttttgacattggtgtcaaccttagacaatacttttgcaactctggtctcaatttctttaagacagtcaccctgatcttctgatagtaactcttcaactccatcaggctctattgactccttttttatttggatcaatgatgacaacctcttcagaatcttgaaatttacctcctttggatctaacttgtatttacccttagttttgaaatgttcaatcatttccacaacaacagcagacacaatggaagagtaatcatattcagtgatgacctcaccaacttcattgagttttggaaccaccacacttttgttgctggacatatccaaggctgtacttgtgaaggagggagtcatagggtcacaaggaagcaggggtttcacttccaatgagctactgttaaatagtgatagacaaacactaagtacatccttattcaaccccggccttccctcacatttggattccagctttttaccaagtagtctctctatatcatgcaccatcttctcttcttcctcagtaggaagttccatactattagaagggttgaccaagactgaatcaaactttaactttggttccaagaacttctcaaaacatttgatttgatcagttaatctatcaggggtttctttggttataaaatggcataaataggagacattcaaaacaaacttaaagatcttagccatatcttcctctctggagttgctgagtaccagaagtatcaaatcatcaataagcattgctgtctgccattctgaaggtgttagcataacgactttcaatttctcaaacaattctttaaaatgaacttcatttacaaaggccataatgtaatatctaaagccttgcaagtaaacttgaatacgcttggaaggggtgcacagtatgcagagaataagtcgtctgagtaaatcagaaacagaatccaagaggggttgggacataaagtccaaccaggataacatctccacacaagtcctttgaatcacatctgcactaaagatcggtaagaaaaatctcttgggatcacagtaaaaagacgcttttgtttcatacaaacccccacttttggatctataagcaacagcataacacctggacctctcccctgtcttctggtacagtagtgtgagagaacctccttctccaaatcgctggaagaaaacttcgtcacagtaaaccttcccataaaactcatcagcattgttcaccttcatcttaggaactgctgctgtcttcatgctattaatgagtgacaaactcaaacttgacaatgttttcagcaattcctcaaactcactttcgcccatgatggtataatcaggctgccctcttcctggcctacccccacacatacactgtgactttgtcttgtattgaagacagggtttagcaccccattcatctaacactgatgttttcagattgaagtaatattcaacatcaggttcccgtagaagagggagaatgtcatcaaggggaagttcaccacagaccgagctcagtctcttcttagccttctctaaccagttggggtttttaatgaattttttagtgatttgttccatcaggaagtcgacattaatcaacctgtcatttacagacggtaacccttgcattaggagcacctctctgaacacagcacctggagaagacttgtccaagtcacacaaaatgttgtacatgataaggtccagaaccaacatggtgttcctccttgtgttaaaaaccttttgagacttaattttgttgcatattgaaagtactctaaaatattctctgctttcagttgatgaatgcttgacctcagattgcctgagttggcctattatgcccaaaatgtgtactgagcaaaactcacataatctgatttctgatttaggtacatctttgacagaacattggataaattcatggttctgaagtctagaaatcatatcttccctatctgtagcctgcagtttcctatcgagttgaccagcaagttgcaacattttaaattgctgaaagatttccatgatttttgttctacattgatctgttgtcagtttattattaatgccagacattaatgccttttccaacctcactttgtaaggaagtcccctttcctttacagcaagtagtgactccagaccgagactctgattttctaaggatgagagggaacttataaggcgttcgtactccaactcctcaacttcttcaccagatgtccttaatccatccatgagttttaaaagcaaccaccgaagtctctctaccacccaatcaggaacaaattctacataataactggatctaccgtcaataacaggtactaaggttatgttctgtctcttgagatcagaactaagctgcaacagcttcaaaaagtcctggttgtatttcttctcaaatgcttcttgactggtcctcacaaacacttccaaaagaatgaggacatctccaaccatacagtaaccatctggtgtaacatccggcaatgtaggacatgttactctcaactccctaaggatagcattgacagtcatctttgtgttgtgtttgcaggagtgtttcttgcatgaatccacttccactagcatggacaaaagcttcaggccctctatcgtgatggccctatctttgacttgtgcaagaacgttgtttttctgttcagatagctcttcccattcgggaacccattttctgactatgtctttaagttcgaaaacgtattcctccatgatcaagaaatgcctaggatcctcggtgcg